(19) United States
(12) Patent Application Publication
(10) Pub. No.: US 2019/0192565 A1
(43) Pub. Date: Jun. 27, 2019

(54) METHODS TO INDUCE CONVERSION OF REGULATORY T CELLS INTO EFFECTOR T CELLS FOR CANCER IMMUNOTHERAPY

(71) Applicant: Dana-Farber Cancer Institute, Inc., Boston, MA (US)

(72) Inventors: Harvey Cantor, Boston, MA (US); Hye-Jung Kim, Brookline, MA (US)

(73) Assignee: Dana-Farber Cancer Institute, Inc., Boston, MA (US)

(21) Appl. No.: 15/578,871

(22) PCT Filed: Jun. 3, 2016
(86) PCT No.: PCT/US16/35692

§ 371 Date: Dec. 1, 2017

Related U.S. Application Data
(60) Provisional application No. 62/170,379, filed on Jun. 3, 2015.
(60) Provisional application No. 62/337,193, filed on May 16, 2016.

(51) Int. Cl. A61K 35/17; C12N 5/0783; C07K 16/18; A61P 35/00; A61P 37/02; C12Q 1/02; G01N 15/14

(52) U.S. Cl. CPC A61K 35/17; C12N 5/0636; C07K 16/18; A61K 45/06; A61P 37/02; C12Q 1/025; G01N 15/14; A61P 35/00

(57) ABSTRACT

Provided by the disclosure are methods for modulating differentiation of regulatory T cells (e.g., CD4⁺ or CD8⁺ regulatory T cells). In some embodiments, methods include contacting regulatory T cells with an agent that decreases Helios activity and/or Helios expression.

### RELATED APPLICATIONS

This application is a national stage filing under 35 U.S.C. § 371 of international application number PCT/US2016/035692, filed Jun. 3, 2016, which was published under PCT Article 21(2) in English and claims priority under 35 U.S.C. § 119(e) to U.S. provisional application No. 62/170,379, filed Jun. 3, 2015 and U.S. provisional application No. 62/337,193, filed May 16, 2016, the contents of each of which are incorporated herein by reference in their entireties.

### FEDERALLY SPONSORED RESEARCH

This invention was made with government support under RO1AI037562 awarded by the National Institutes of Health. The government has certain rights in the invention.

### BACKGROUND OF INVENTION

Regulatory T cells (Treg) are critically important for the maintenance of immune homeostasis. A central aspect of Treg biology is maintenance of inhibitory activity and anergy in the face of vigorous immune and inflammatory responses, particularly in view of their self-reactive T cell receptor (TCR) repertoire. Stable expression of a specialized suppressive genetic program by Treg in a changing immunologic environment is essential for maintenance of self-tolerance by these cells. Transcription factors (TF) responsible for maintaining the stable differentiated immunosuppressive phenotype of Treg likely contribute to this process.

A number of reports have documented the presence of Treg within human tumor tissue, and in one of these studies the number of Treg also showed a clear negative correlation with survival (Zou, W. 2006. Regulatory T cells, tumour immunity and immunotherapy. Nat Rev Immunol 6:295-307; Beyer, M. et al. 2006. Regulatory T cells in cancer. Blood 108:804-81 1; Curiel, T. J. et al. 2004. Specific recruitment of regulatory T cells in ovarian carcinoma fosters immune privilege and predicts reduced survival. Nat Med 10:942-949; Mourmouras, V. et al. 2007. Evaluation of tumour-infiltrating CD4+CD25+FOXP3+ regulatory T cells in human cutaneous benign and atypical naevi, melanomas and melanoma metastases. Br J Dermatol 157:531-539; Viguier, M. et al. 2004. Foxp3 expressing CD4+CD25(high) regulatory T cells are overrepresented in human metastatic melanoma lymph nodes and inhibit the function of infiltrating T cells. J Immunol 173:1444-1453). Thus, Treg may play a major role in preventing the development of effective anti-tumor immunity. Modulation of TF activity to control Treg differentiation therefore represents a potential therapeutic strategy for the treatment of certain diseases (e.g., cancer) and autoimmune conditions. However, little is understood about the number, identity and biological roles of TF that control Treg differentiation.

### SUMMARY OF INVENTION

The disclosure relates to methods and compositions for modulating the differentiation of Treg. The disclosure is based, in part on the surprising discovery that the T cell specific transcription factor (TF) Helios, a member of Ikaros family, is expressed by both CD4⁺ and CD8⁺ regulatory lineages (e.g., CD4⁺ Treg and CD8⁺ Treg cells) and that modulation of Helios expression or activity plays a role in controlling differentiation of these Treg cells.

Accordingly, in some aspects the disclosure provides a method for inducing differentiation of a regulatory CD4⁺ T (CD4⁺ Treg) cell to a CD4⁺ effector T cell, the method comprising contacting the CD4⁺ Treg with an agent that decreases Helios activity and/or Helios expression. In some embodiments, the CD4⁺ Treg cell is FoxP3⁺ and CD25⁺.

In some embodiments, the agent that decreases Helios activity and/or expression in a CD4⁺ Treg is selected from the group consisting of peptide, polypeptide, small molecule, antibody, and RNAi molecule. In some embodiments, the agent is an antibody. In some embodiments, the antibody is selected from the group consisting of anti-GITR, anti-OX-40, anti-CD47, anti-4-1BB, anti-Nrp-1, and anti-CD73 antibody. In some embodiments, the small molecule is a zinc finger protein inhibitor.

In some embodiments, the CD4⁺ effector T cell expresses one or more effector cytokines. In some embodiments, the effector cytokines are selected from the group consisting of tumor necrosis factor alpha (TNF-α), interferon-γ (IFN-γ), interleukin-17 (IL-17), interleukin-2 (IL-2), and Granzyme B.

In some aspects, the disclosure provides a method for inducing differentiation of a regulatory CD8⁺ T (CD8⁺ Treg) cell to a CD8⁺/PD1⁺/TIM3⁺ T cell, the method comprising contacting the regulatory T cell with an agent that decreases Helios activity and/or Helios expression. In some embodiments, the CD8⁺ Treg cell is Kir⁺.

In some embodiments, the agent that decreases Helios activity and/or expression in a CD8⁺ Treg is selected from the group consisting of peptide, polypeptide, antibody small molecule and RNAi molecule. In some embodiments, the agent is an antibody. In some embodiments, the antibody is selected from the group consisting of anti-Kir, anti-Ly49F, or a bispecific anti-CD8/anti-Kir antibody. In some embodiments, the small molecule is a zinc finger protein inhibitor, or a Stat5b inhibitor.

In some embodiments, the CD8⁺/PD1⁺/TIM3⁺ T cell express increased levels of BLIMP-1 transcription factor when compared to wild-type CD8⁺ regulatory T cells.

In some aspects, methods and compositions described by the disclosure are useful for the treatment of certain diseases (e.g., cancer and autoimmune diseases). In some aspects, the disclosure provides a method for treating cancer in a subject, the method comprising administering to the subject an agent that induces differentiation of regulatory CD4⁺ T (CD4⁺ Treg) cells to CD4⁺ effector cells by decreasing Helios activity and/or Helios expression.

In some aspects, the disclosure provides a method for treating cancer in a subject, the method comprising administering to the subject an agent that induces differentiation of regulatory CD8⁺ T (CD8⁺ Treg) cells to CD8⁺/PD1⁺/TIM3⁺ T cells by decreasing Helios activity and/or Helios expression.

In some embodiments, the cancer is selected from the group consisting of: brain cancer, breast cancer, bladder cancer, pancreatic cancer, prostate cancer, liver cancer, kidney cancer, lymphoma, leukemia, lung cancer, colon cancer ovarian cancer, gastric cancer, cervical cancer, gliomas, head and neck cancers, esophagus cancer, gall bladder cancer, thyroid cancer, and melanoma.

In some embodiments, the method further comprises administering to the subject an immune checkpoint inhibitor. In some embodiments, the immune checkpoint inhibitor is an antibody. In some embodiments, the immune checkpoint inhibitor is selected from the group consisting of a PD-1 antagonist, a TIM-3 antagonist, and a TIGIT antagonist.

In some embodiments, the method further comprises administering to the subject a chemotherapeutic agent.

In some aspects, the disclosure provides a method for inhibiting differentiation of a CD4⁺ regulatory T cell to a CD4⁺ effector T cell, the method comprising contacting the CD4⁺ regulatory T cell with an agent that increases Helios activity and/or Helios expression.

In some aspects, the disclosure provides a method for inhibiting differentiation of a CD8⁺ regulatory T cell to a CD8⁺/PD1⁺/TIM3⁺ T cell, the method comprising contacting the CD8⁺ regulatory T cell with an agent that increases Helios activity and/or Helios expression.

In some aspects, the disclosure provides a method for treating autoimmune disease in a subject, the method comprising administering to the subject an agent that inhibits differentiation of CD4⁺ regulatory T cells to CD4⁺ effector cells, by increasing Helios activity and/or Helios expression.

In some aspects, the disclosure provides a method for treating autoimmune disease in a subject, the method comprising administering to the subject an agent that inhibits differentiation of CD8⁺ regulatory T cells to CD8⁺/PD1⁺/TIM3⁺ T cells, wherein the agent increases Helios activity and/or Helios expression.

Methods described by the disclosure are, in some embodiments, useful for identifying agents that modulate differentiation of Treg (e.g., CD4⁺ Treg or CD8⁺ Treg). In some aspects, the disclosure provides a method for identifying candidate compounds for modulating Helios activity and/or Helios expression, the method comprising: contacting a regulatory T cell with a test compound; measuring Helios activity level and/or Helios expression level in the cell; identifying the test compound as a candidate compound for modulating Helios activity and/or Helios expression if the Helios activity level and/or Helios expression level is increased or decreased relative to a control cell that has been treated with a compound known to not modulate Helios activity level and/or Helios expression level.

In some aspects, the disclosure provides a method for identifying candidate compounds for decreasing Helios activity and/or Helios expression, the method comprising: contacting a regulatory T cell with a test compound; measuring Helios activity level and/or Helios expression level in the cell; identifying the test compound as a candidate compound for decreasing Helios activity and/or Helios expression if the Helios activity level and/or Helios expression level is decreased relative to a control cell that has been treated with a compound known to not decrease Helios activity level and/or Helios expression level.

In some embodiments, the method further comprises measuring FoxP3 activity level and/or FoxP3 expression level.

In some embodiments, the test compound is selected from the group consisting of peptide, polypeptide, antibody, small molecule, RNAi molecule and CRISPR/Cas molecule.

In some embodiments, the measuring is performed by a protein-based screening method. In some embodiments, the protein-based screening method comprises: contacting the cell with a detectable antibody targeting Helios; contacting the cell with a detectable antibody targeting FoxP3; contacting the cell with at least one detectable antibody targeting an effector cytokine; and, detecting the level of the detectable antibodies.

In some embodiments, the at least one detectable antibody targeting an effector cytokine targets TNF-α, IFN-γ, IL-17, IL-10 or IL-2. In some embodiments, detectable antibodies are a fluorescently labeled. In some embodiments, the detecting is performed by flow cytometry.

Each of the embodiments and aspects of the invention can be practiced independently or combined. Also, the phraseology and terminology used herein is for the purpose of description and should not be regarded as limiting. The use of “including”, “comprising”, or “having”, “containing”, “involving”, and variations thereof herein, is meant to encompass the items listed thereafter and equivalents thereof as well as additional items.

These and other aspects of the inventions, as well as various advantages and utilities will be apparent with reference to the Detailed Description. Each aspect of the invention can encompass various embodiments as will be understood.

All documents identified in this application are incorporated in their entirety herein by reference.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIGS. 1A-1C show specific expression of Helios by CD44⁺CD122⁺Ly49⁺CD8 Treg. FIG. 1A shows cDNA from highly purified (>99%) CD44⁺CD122⁺Ly49⁺ and CD44⁺CD122⁺Ly49⁻ CD8 cells from spleen of B6 mice were subjected to DNA microarray analysis. Genes were up-regulated (90 genes) or down-regulated (33 genes) in CD44⁺CD122⁺Ly49⁺CD8 cells by >2 fold change. Expression of Helios by CD44⁺CD122⁺Ly49⁺CD8 T cells, but not CD44⁺CD122⁺Ly49⁻ CD8 T cells, was verified according to FACS analysis. Expression of Helios in the total CD8 T cell pool is shown at the bottom right. FIG. 1B shows stable expression of Helios in CD8 Treg during homeostatic expansion. K^{b-/-}D^{b-/-} mice were depleted of NK cells by injection of anti-NK1.1 Ab followed by sub lethal (600 rads) irradiation. 24 h later, CFSE labeled CD122⁺Ly49⁺, CD122⁺Ly49 and CD122⁻CD8 T cells were transferred intravenously. 5 days later, proliferation and expression of Helios by transferred CD8 cells was analyzed by FACS. FIG. 1C shows FoxP3-GFP reporter mice were analyzed for Helios expression by FoxP3⁺CD4 T cells and FoxP3 and Helios expression by Ly49⁺CD8 T cells.

FIGS. 2A-2I show Helios^{-/-} mice develop an autoimmune phenotype. FIG. 2A shows comparison of activated CD4 and CD8 T cells (CD44⁺CD62L^{lo}), GC B (B220⁺Fas⁺) and T_{FH} (CD4⁺PD-1⁺CXCR5⁺) cells in spleens from (5 mo old) Helios^{+/+} and Helios^{-/-} mice (n=3-6). FIG. 2B shows microscopy (200×) of representative hematoxylin and eosin staining of salivary gland, liver, lung, pancreas, kidney sections from (7 mo old) Helios^{+/+} and Helios^{-/-} mice. FIG. 2C shows generation of autoantibodies specific for multiple self-antigens were compared using sera from 7 mo old Helios^{+/+} and Helios^{-/-} mice (n=7-10). FIG. 2D shows kidney pathology assessment and quantification by PAS staining (400×) and deposition of IgG (400×) in glomeruli. FIG. 2E shows T and NK cell depleted BM cells from Helios^{+/+} and Helios^{-/-} mice were transferred to lethally irradiated (900 rads) Rag2^{-/-} hosts. 9 weeks later, mice were analyzed for immune phenotype. Spleens from BM chimera are shown. FIG. 2F shows flow cytometric analysis of CD44 and CD62L expression in spleen CD4⁺ and CD8⁺ T cells and FIG. 2G shows autoantibodies from Rag2^{-/-} hosts reconstituted with Helios^{+/+} and Helios^{-/-} hematopoietic cells. FIG. 2H shows viral infection induces early autoimmune development in Helios deficient mice. 2 mo and 6 mo old Helios WT and KO mice were infected i.p. with 2×10⁵ plaque forming units (PFU) LCMV-Armstrong. 30 days later, spleen cells were analyzed for the formation of GC B and T_{FH} cells. FIG. 2I shows kidney sections from these virus infected mice were analyzed for IgG deposition and IgG⁺ areas in glomeruli were depicted (n=4).

FIGS. 3A-3G show Helios deficiency in both CD4 and CD8 Treg contributes to the perturbed immune homeostasis. FIG. 3A shows thymic negative selection is not defective in Helios^{-/-} mice. Flow cytometric analysis of Helios^{+/+} or Helios^{-/-} thymocytes from BM chimera reconstituted with Helios WT and KO BM cells for the percentage of CD4^{dull}CD8^{dull} (DP^{dull}) subset and active Caspase-3, PD-1 and CD5 and CD69 expression within apoptotic Cd4^{lo}CD8^{lo} cells at 5 weeks after BM reconstitution (n=4-5). FIG. 3B shows lethally irradiated WT B6 or RIP-mOVA transgenic mice were reconstituted with BM cells from Helios^{+/+} OT-II or Helios^{-/-} OT-II mice that were depleted of NK1.1+, TCR+, CD4+ and CD8+ cells. Development of OT-II cells (Va2⁺Vb5⁺) was analyzed 8 weeks after reconstitution. Percentage of OT-II cells in total thymocytes (upper panel) and CD4 SP, DP and CD8 SP thymocytes within Va2⁺V35⁺ thymocytes (lower panel) are shown (n=4). FIG. 3C shows lethally irradiated Rag2^{-/-} mice were reconstituted with hematopoietic progenitors from Helios^{+/+}, Helios^{-/-}, CD4^{-/-}/Helios^{-/-} (50:50) and CD8^{-/-}/Helios^{-/-} (50:50) mice. Flow cytometric plots for activated CD4 (CD44⁺CD62L^{lo}CD4⁺) cells and numbers in spleen are shown. FIG. 3D shows analysis of immune cell infiltration into various organs from Rag2 mice reconstituted with hematopoietic precursors described in b). Intensity of immune cell infiltration into peripheral organs was quantified by scoring tissue sections by >4 (mostly severe), 2-3 (severe), 1 (mild) and 0 (none). FIG. 3E shows Helios deficiency selectively in FoxP3⁺ cells contributes to the development of autoimmune disease. Lethally irradiated Rag2−/− mice were reconstituted with hematopoietic progenitors from Helios+/+, Helios−/−, Scurfy, Helios+/+/Helios−/− (50:50), Helios+/+/Scurfy (50:50), Helios−/−/Scurfy (50:50) and Heliosfl/fl/CD4-Cre/Scurfy mice. Activation of conventional CD4 T cells in spleen was analyzed by measuring the percentage of CD44^{hi}CD62L^{lo} cells within FoxP3⁻CD4⁺ cells. FIG. 3F shows intensity of immune cell infiltration into peripheral organs in BM chimeras described in FIG. 3F was quantified by scoring levels of immune cell infiltration by >4 (mostly severe), 2-3 (severe), 1 (mild) and 0 (none). FIG. 3G shows surface phenotype of FoxP3⁺CD4 cells in spleens from BM chimeras described in FIG. 3F) was analyzed by levels of FR4 and CD73 expression (n=4-6).

FIGS. 4A-4H. Expression of Helios is important for Treg function. FIG. 4A shows expression of Helios is important for Treg function. Rag2^{-/-} hosts received sort-purified CD4 T cells (Teff: CD44^{lo}CD62L^{hi}, CD4 Treg: CD3⁺CD4⁺CD25⁺) from defined donor mouse strains. Recipients were examined for changes in weight, microscopic intestine pathology (n=4). FIG. 4B shows Rag2^{-/-} hosts received sort-purified Teff cells (CD44^{lo}CD62L^{hi}, CD45.1) and CD4 Treg (CD3⁺CD4⁺YFP⁺) from FoxP3^{YFP}-Cre or Heliosfl/fl/FoxP3^{YFP}-Cre mice. Recipients were examined for changes in weights and survival (n=4). FIGS. 4C and 4D show Levels of FoxP3 expression by FoxP3⁺CD4 cells in spleens from indicated hosts was evaluated. FIG. 4E shows spleen cells from Rag2^{-/-} hosts were analyzed for the frequency and numbers of CD11b⁺Gr1⁺ cells. FIG. 4F shows defective suppressive function of Helios^{-/-} CD8 Treg. WT B and CD25-depleted CD4 T cells were transferred into Rag2^{-/-} hosts along with Ly49⁺ or Ly49⁻ CD8 T cells from either Helios^{+/+} or Helios^{-/-} mice. Rag2^{-/-} adoptive hosts were immunized with NP₁₉-KLH in CFA at day 0 and reimmunized with NP₁₉-KLH in IFA at day 10. Primary and secondary NP specific IgG1 responses were measured using serum prepared at day 10 and 15 (n=3). FIG. 4G shows WT B and CD25-depleted CD4 T cells were transferred into Rag2^{-/-} hosts along with Ly49⁺ or Ly49⁻ CD8 T cells from Rag2^{-/-} BM chimera reconstituted with Helios^{+/+} or Helios^{-/-} hematopoietic cells. Rag2^{-/-} adoptive hosts were immunized with NP₁₉-KLH in CFA at day 0 and reimmunized with NP₁₉-KLH in IFA at day 10. Secondary IgG response to NP is shown (n=3).

FIGS. 5A-50 show Helios dependent molecular pathways contribute to the CD4 and CD8 Treg integrity. FIG. 5A shows distribution of genome wide Helios binding sites in FoxP3⁺CD4 and Ly49⁺CD8 Treg; FIG. 5B shows the number of Helios target genes and overlapping Helios binding sites in CD4 and CD8 Treg; FIG. 5C shows DNA motif analysis of Helios bound regions; FIG. 5D shows representative molecular pathways in CD4 and CD8 Treg that are contributed by Helios target genes; and, FIG. 5E shows ChiP-seq analysis of the binding of Helios and modified histones at Birc1, Bag1, NFAT1, Jak and Stat5b in CD4 and CD8 Treg. Vertical lines in gene diagrams (bottom) indicate exons. FIG. 5F shows BM chimeras were generated by reconstituting lethally irradiated Rag2^{-/-} mice with Helios^{+/+}, Helios^{-/-}, Helios^{fl/fl}/CD4-Cre or Helios^{fl/fl}/FoxP3YFP-Cre mice. 6-8 wks after BM reconstitution, IL-2 responsiveness of FoxP3+CD4 cells from spleens of each group was tested. Representative histograms for the expression of p-Stat5b from two independent experiments are shown in FIGS. 5G-5I. CD4 Treg from WT (CD45.1) and IKZF^{fl/fl}/CD4-Cre (CD45.2) mice were cotransferred into Rag2−/−γc−/− mice. FoxP3⁺CD4 Treg from spleens of recipients were analyzed for the numbers, apoptosis and surface phenotype at 5 days after transfer (n=4). FIG. 5J shows 1×10⁶ OT-II cells were transferred into Rag2^{-/-} hosts followed by immunization with OT-II peptide (10 g) in CFA. Sort-purified CD25⁺CD4⁺ T cells (2×10⁵) from CD45.1⁺ Helios^{+/+} or CD45.2⁺ Helios^{-/-} mice were transferred into these Rag2^{-/-} hosts. 5 days after CD4 Treg transfer, spleen cells from Rag2^{-/-} hosts were analyzed for CD4⁺ T cells of OT-II (V_{β}5⁺), CD45.1 or CD45.2 phenotype and levels of Helios, FoxP3 and RORγt expression. Cytokine expression by Helios^{+/+} and Helios^{-/-} CD4 Treg upon in vitro restimulation with PMA and ionomycin was assessed by intracellular cytokine analysis. Levels of cytokines IFNγ, IL-17A and TNFα and TF RORγt by Helios^{+/+} and Helios^{-/-} CD4 Treg are shown after gating on FoxP3⁺CD4 cells. FIG. 5K shows Ly49⁺CD8 Treg from Helios WT (CD45.1⁺) and Helios KO (Heliosfl/fl/CD4-Cre) mice were transferred into Rag2^{-/-}Prf^{-/-} mice along with OT-II cells (Va2⁺Vβ5⁺) followed by immunization with OT-II peptides (20 ug) in IFA. 5 days later, the percentage of Ly49⁺CD8 cells from each origin and levels of apoptosis in spleens were analyzed. FIG. 5L shows Lethally irradiated Rag2 mice were reconstituted with hematopoietic precursors from Helios^{+/+} or Helios^{-/-} mice (n=3-4). At the time point of robust autoimmune progression (˜8 wks after BM reconstitution), Ly49⁺CD8 T cells from spleens of these mice were analyzed for the expression of PD-1, Lag3, TIM3 and CD127. FIG. 5M shows FACS-sorted Ly49⁺CD8 cells (>99%) were transferred into Rag2^{-/-} hosts along with OT-II cells followed by immunization with OT-II peptides in IFA. After 12 days, CD8 T cells in these adoptive hosts were analyzed for PD-1 and TIM3 expression. FIG. 5N shows CD8 T cells recovered from Rag2^{-/-} hosts that were transferred with B, CD4 and Ly49⁺10 CD8 cells from Helios^{+/+} or Helios^{-/-} mice as described above, were analyzed for their phenotype. Recovered CD8 cells from spleen were sorted based on PD-1 expression and analyzed for Blimp-1 expression. Graph shows fold increase of Blimp-1 expression over PD-1-Helios WT CD8 cells. FIG. 5O shows the numbers of Ly49⁺CD8 cells recovered from spleens of Helios^{+/+} or Helios^{-/-} BM chimeras (c) or Rag2^{-/-} hosts (d) were analyzed.

FIG. 6 shows that Ly49⁺CD8 cells arise early in life and increase in frequency and numbers during aging. WT B6 mice at the indicated ages were analyzed for the percent of CD122⁺Ly49⁺ cells within CD3⁺CD8⁺ cells and total numbers recovered per spleen.

FIG. 7 shows the percentages of CD122⁺Ly49⁺CD8 and FoxP3⁺CD4 cells within CD8 T and CD4 T cells, respectively, from spleen of Helios^{+/+} and Helios^{-/-} mice.

FIG. 8 shows the analysis of LCMV-gp33 specific CD8 cells and virus titer after LCMV-Arm infection in Helios WT an Helios KO mice. 2 mo old Helios WT and KO mice were infected i.p. with 2×10⁵ pfu LCMV-Arm. Gp33 specific CD8+ T cells and virus titer in the blood were analyzed at day 5, 8 and 12 after infection.

FIG. 9 shows that thymic negative selection is not defective in Helios^{-/-} mice. Flow cytometric analysis of Helios^{+/+} or Helios^{-/-} thymocytes (8 wks old) for the percentage of CD4dullCD8dull (DPdull) subset and active Caspase-3 and CD69 expressing DPdull thymocytes. Representative plots and cell numbers are shown (n=4).

FIG. 10 shows that thymic negative selection of self-reactive cells is not impaired in the Helios deficiency. Lethally irradiated WT B6 or RIP-mOVA transgenic mice were reconstituted with BM cells from Helios^{+/+} OT-II or Helios^{-/-} OT-II mice that were depleted of NK1.1⁺, TCR⁺, CD4⁺ and CD8⁺ cells. Development of OT-II cells (Va2⁺V35⁺) was analyzed 8 weeks after reconstitution. Percentage of Va2⁺V35⁺ cells within total splenocytes and development of OT-II cells within Va2⁺V35⁺ splenocytes are shown (n=4).

FIG. 11 shows that Helios deficiency does not impair thymic generation of self-reactive FoxP3⁺CD4 Treg. Lethally irradiated WT B6 or RIP-mOVA transgenic mice were reconstituted with BM cells from Helios^{+/+} OT-II or Helios^{-/-} OT-II mice that were depleted of NK1.1+, TCR+, CD4+ and CD8+ cells. Percentage and number of FoxP3⁺CD4 cells in OT-II thymocytes in a representative experiment were analyzed.

FIG. 12 shows the analysis of HY-TCR KI mice, revealing that Helios deficiency does not impact negative selection of self-reactive HY⁺CD8 T cells in thymus. HY TCR KI mice in Helios^{+/+} and Helios^{-/-} background were compared for the DP^{dull} thymocytes undergoing apoptosis and development of HY⁺ SP T cells in female and male mice. Representative data from two independent experiments is shown.

FIG. 13 shows that the Helios deficiency does not impair MTV-mediated deletion of TCR Vβ5⁺ CD4 cells. Percentage of TCR Vβ5⁺ CD4 cells in thymus and spleen was compared between Helios^{+/+} and Helios^{-/-} mice. Percentage of TCR V136 serves as a reference (n=4-5).

FIG. 14 shows the selective deletion of Helios in CD4 and CD8 Treg by mixed BM reconstitution. Lethally irradiated Rag2^{-/-} mice were reconstituted with hematopoietic progenitors from Helios^{+/+}, Helios^{-/-}, CD4^{-/-}/Helios^{-/-} (50:50) and CD8^{-/-}/Helios^{-/-} (50:50) mice. Helios expression by FoxP3⁺CD4 Treg and Ly49⁺CD8 Treg were analyzed with spleen cells from each BM chimera 8 weeks after BM reconstitution.

FIG. 15 shows that Helios^{+/+} and Helios^{+/+}/Helios^{-/-} BM chimera display similar levels of CD4 T cell activation. Lethally irradiated Rag2^{-/-} mice were reconstituted with hematopoietic progenitors from Helios^{+/+}, Helios^{-/-} and Helios^{+/+}/Helios^{-/-} (50:50) mice. Helios expression by FoxP3⁺CD4 Treg and Ly49⁺CD8 Treg were analyzed with spleen cells from each BM chimera (upper panel). Percentage of activated CD4 cells were analyzed in these BM chimera (lower panel). Histological analyses confirmed this finding.

FIGS. 16A-16C show Helios deficiency selectively in FoxP3⁺ cells contributes to the development of autoimmune disease. Lethally irradiated Rag2^{-/-} mice were reconstituted with hematopoietic progenitors from Helios^{+/+}, Helios^{-/-}, Scurfy, Helios^{+/+}/Helios⁻/₋ (50:50), Helios^{+/+}/Scurfy (50:50), Helios^{-/-}/Scurfy (50:50) and Helios^{fl/fl}/CD4-Cre/Scurfy mice. FIG. 16A shows the change of body weight was monitored. The percentage weight change 7 weeks after reconstitution is shown (n=4-5). FIG. 16B shows intensity of immune cell infiltration into peripheral organs was quantified by scoring levels of immune cell infiltration by >4 (mostly severe), 2-3 (severe), 1 (mild) and 0 (none). FIG. 16C shows percentage of FoxP3+ cells within CD4 cells in spleen are shown (n=4-6).

FIG. 17 presents the sorting strategy for CD4 Treg isolation from Helios^{+/+}/FoxP3YFP-Cre and Helios^{fl/fl}/FoxP3YFP-Cre mice. Expression pattern of CD25 and FoxP3 (YFP) in CD4 cells (left). Gating for YFP⁺ (FoxP3⁺) cells in CD4 cells from Helios^{+/+}/FoxP3YFP-Cre and Helios^{fl/fl}/FoxP3YFP-Cre mice (middle). Purity of YFP⁺ cells after sorting (right).

FIGS. 18A-18C show the development of autoimmune disease in Helios^{fl/fl}/FoxP3-Cre BM chimeras. NK1.1⁺, CD4⁺, CD8⁺ TCR⁺ depleted BM cells from WT or Helios^{fl/fl}/FoxP3-Cre mice were transferred into lethally irradiated Rag2^{-/-} mice before analysis for signs of autoimmune disease 6 weeks after BM transfer. FIG. 18A shows immune cell infiltration into multiple organs was analyzed. FIG. 18B shows percentage of activated CD4 cells and FIG. 18C shows expression of FR4 and CD73 by FoxP3⁺CD4 T cells was analyzed with spleen cells from these BM chimeras (n=9-10).

FIG. 19 shows the Chip-seq analysis of the binding of Helios, H3K27ac and H3K27me3 at IL-2Ra and FoxP3 gene loci.

FIG. 20 shows the IL-2 responsiveness of CD4 Treg from Helios^{+/+}, Helios−/− and Heliosfl/fl/CD4-Cre and Heliosfl/fl/FoxP3-Cre mice. a) Spleen cells from Helios^{+/+}, Helios−/− and Heliosfl/fl/CD4-Cre BM chimera were stimulated with IL-2 (100 ng/ml) in vitro and levels of p-STAT5 expression was measured. The figure shows basal p-STAT5 expression (red line, no IL-2 stimulation) and p-STAT5 expression after IL-2 (blue line) stimulation.

FIGS. 21A-21D show that Helios-deficient CD4 Treg in the inflammatory condition display non-anergic phenotye. FIG. 21A shows FACS sorted naïve CD4 T cells (CD44^{lo}CD62L^{hi}, CD45.1⁺) were transferred into Rag2^{-/-} hosts. At the time point when mice showed weight loss ˜10% of original, CD4 Treg from Helios^{fl/fl} or Helios^{fl/fl}/CD4-Cre mice were transferred and development of wasting disease monitored. FIG. 21B shows, levels of FoxP3 expression in mice sacrificed at week 10 after transfer and the percentage of FoxP3⁺CD4 T cells; FIG. 21C shows the ratio between FR4^{hi}CD73^{hi} vs. FR4^{lo}CD73^{lo} within FoxP3⁺CD4 cells were analyzed. FIG. 21D shows effector cytokine production by FoxP3⁺CD4 Treg was analyzed after in vitro restimulation of spleen cells with PMA and ionomycin. Left panel: IL-17 and IFNg production by FoxP3⁺CD4 Treg recovered from Rag2^{-/-} hosts transferred with Helios^{fl/fl} CD4 or Helios^{fl/fl}/CD4-Cre Treg. Right panel: Cytokine production by FoxP3⁺CD4 Treg from Rag2^{-/-} hosts transferred with Helios^{fl/fl}/CD4-Cre CD4 Treg was analyzed by dividing cells according to FR4 and CD73 expression (FR4^{hi}CD73^{hi} and FR4^{lo}CD73^{lo} cells).

FIG. 22 shows that Helios deficient CD4 Treg acquire non-anergic phenotype in the inflammatory environment. Helios^{+/+} and Helios^{-/-} mice were infected i.p. with 2×10⁵ pfu LCMV-Armstrong. At day 8 after infection, spleen cells were harvested and the surface phenotype of FoxP3⁺CD4 Treg in spleen was analyzed.

FIG. 23 shows that the expression of Helios is important for the suppressive activity of FoxP3⁺CD4 Treg. Rag2^{-/-} hosts received native CD4 cells (CD45.1) and CD4 Treg (YFP⁺) from defined donor mouse strains were examined for surface phenotype of CD4 Treg with FR4 and CD73 expression.

FIGS. 24A-24B show the reduced FoxP3 expression and cytokine secretion by Helios deficient CD4 Treg. FIG. 24A shows FoxP3 expression by CD4 T cells was compared in splenocytes from Helios WT and KO mice in age 8 months. Histogram in the right shows the comparison of FoxP3 expression after gating on FoxP3+CD4+ cells. FIG. 24B shows spleen cells from 8 mo old Helios WT and KO mice were stimulated in vitro with PMA and ionomycin and cytokine expression was measured. FoxP3+CD4+ cells are gated and percentage of cytokine secreting cells within FoxP3⁺ cells is shown.

FIG. 25 shows the expression of PD-1 and TIM3 by FoxP3⁺CD4 Treg. Lethally irradiated Rag2−/− mice were reconstituted with hematopoietic progenitors from Helios^{+/+}, Helios^{-/-}, Scurfy, Helios^{+/+}/Helios^{-/-}, Helios^{+/+}/Scurfy (50:50), Helios^{-/-}/Scurfy (50:50) and Helios^{fl/fl}/CD4-Cre/Scurfy mice. 7 weeks after reconstitution, The number of FoxP3⁺CD4 Treg and surface expression of PD-1 and TIM3 was analyzed.

FIGS. 26A-26C depict a comparison of gene expression between Helios+ and Helios−/lo FoxP3+CD4 Treg. Helios+ and Helios−/lo CD4 Treg were sorted from spleen of WT B6 mice after staining with Abs for CD3, CD4, CD25, ICOS and GITR. FIG. 26A shows a comparison of gene expression between Helios⁺ and Helios^{lo/-} FoxP3⁺CD4 Treg. Helios⁺ and Heliosoi FoxP3⁺CD4 Treg sorted from spleen of WT B6 mice after staining with Abs for CD3, CD4, CD25, ICOS and GITR. FIG. 26A shows ICOS^{hi}GITR^{hi} and ICOS^{lo}GITR^{lo} cells represent Helios⁺ and Helios^{-/lo} cells respectively. FIG. 26B shows a comparison of data generated from a DNA microarray performed using Affymetrix chip. FIG. 26C shows dominant molecular pathways composed of genes that are upregulated in Helios⁺CD4 Treg.

FIG. 27 shows Helios^{-/-} CD4 Treg upregulate RORγt TF and effector cytokines. Helios^{-/-} CD4 Treg exhibit increased apoptosis, decreased regulatory activity and increased Th17 and IFNγ cytokine expression.

FIG. 28 shows a schematic illustration of one embodiment of in vitro screening to identify agents that modulate Helios activity and/or expression levels.

FIG. 29 shows FACS data illustrating that Helios-deficient CD4 Treg show reduced CD73 (ectonuclease) expression during an immune response.

FIG. 30 shows antibody-dependent engagement of CD73 induces down regulation of Helios and FoxP3 Treg.

FIG. 31 shows that intratumoral CD4 Tregs display increased suppressive phenotype. A) Analysis of Helios expression by FoxP3⁺CD4 Treg in spleen, LNs and tumor of B16/F10 melanoma bearing mice. Percent of Helios⁺ FoxP3⁺CD4 Treg is shown (n=3).

FIGS. 32A-32E show that mice with Helios deficiency in CD4 Treg show enhanced anti-tumor immunity. FIG. 32A shows tumor growth and survival of Helios^{fl/fl} and Helios KO mice that were injected s.c. with 2×10⁵ B16/F10 (left panel) or MC38 (right panel) and tumor growth and survival of mice were monitored. FIG. 32B shows enhanced IFNγ production by CD4 and CD8 Teff cells in B16/F10 or MC38 tumors from Helios KO mice. Two weeks after tumor inoculation, lymphocytes were enriched from tumor cell samples and stimulated with PMA and ionomycin in vitro followed by FACS analysis of IFNγ expression by CD4 and CD8 cells. FIGS. 32C and 32D shows tumor growth in Helios WT (FoxP3-Cre) and KO (Helios^{fl/fl}.FoxP3-Cre) mice that were inoculated with 2×10⁵ B16-Ova, and vaccinated with GVAX on days 3, 7 and 9, and tumor growth monitored. FIG. 32E shows TNFα production in effector CD4, CD8 T and FoxP3⁺CD4 cells at day 21.

FIGS. 33A-33D shows unstable phenotype of Helios-deficient CD4 Treg within tumors. Helios^{fl/fl} (WT) and Helios KO mice were injected s.c. with 2×10⁵ MC38. Two weeks later, splenocytes and intratumoral lymphocytes were analyzed. FIG. 33A shows percent of FoxP3⁺ cells within TCR⁺CD4⁺ in spleen and tumor. FIGS. 33B and 33C show that intratumoral but not splenic Helios-deficient CD4 Treg display non-anergic phenotype. FIG. 33B shows data for FoxP3⁺CD4 cells from spleen when tumors were analyzed for expression of FR4 and CD73. FIG. 33C shows IFNγ expression after in vitro restimulation with PMA and ionomycin. FIG. 33D shows effector cytokine TNFα expression by CD4 Treg isolated from tumors in Helios KO mice that were treated as described in FIG. 2E.

FIGS. 34A-34D show isolated function of Helios-deficient CD4 Treg in anti-tumor immunity. FIG. 34A shows tumor volume in Rag2^{-/-} hosts that were transferred with purified CD4 and CD8 T cells (CD4 and CD8 Treg depleted) along with CD4 Treg isolated from Helios WT (FoxP3-Cre) or KO (Helios^{fl/fl}.FoxP3-Cre) mice. Two days later, Rag2^{-/-} hosts were inoculated s.c. with MC38 and tumor growth monitored. FIG. 34B shows data collected 21 days after cell transfer, when cells from spleen and tumor were analyzed for their origin (CD45.1 vs. CD45.2) and FoxP3 expression. FIG. 34C shows IFNγ expression by Helios WT (CD45.2⁺) or KO (CD45.2⁺⁾CD4 Treg recovered from spleens of Rag2^{-/-} hosts after in vitro restimulation. FIG. 34D shows IFNγ expression by intratumoral effector CD4 and CD8 T cells from Rag2^{-/-} hosts that were transferred with Helios WT or KO CD4 Treg after in vitro restimulation.

FIGS. 35A-D show Helios deficiency and Treg to Teff conversion. FIG. 35A shows reduced CD25 and FoxP3 expression by Helios-deficient CD4 Treg upon exposure to IL-4 in vitro. Sorted CD4 Treg from Helios^{+/+} and Helios^{-/-} mice were stimulated for 4-5 days with coated anti-CD3/CD28 Abs in the presence of IL-2 (0-50 ng/ml) and IL-4 (20 ng/ml) before levels of CD25, FoxP3 and IFNγ expression were measured. Representative data from three independent experiments are shown. FIG. 35B shows IFNγ expression levels in Helios-deficient CD4 Treg from cultures of FIG. 35A that were divided into FoxP3^{hi} and FoxP3^{lo}. FIG. 35C shows cell numbers and FoxP3 expression in CD4 Treg from WT B6 mice that were treated in vitro with DMSO or AG-490 in the presence of IL-4 (20 ng/ml) and increasing concentrations of IL-2 (0-50 ng/ml) for 48 hrs. FIG. 35D shows IFNγ production by recovered cells that was analyzed by intracellular cytokine staining. FoxP3⁺CD4 Treg were isolated from spleens of WT B6 and cultured in anti-CD3/CD28-coated wells in the presence of IL-4, increasing concentrations of IL-2 and anti-GITR (DTA-1) or isotype Abs. After 5 days, cells were analyzed for FoxP3 expression and IFNγ production.

FIGS. 36A-36F show that engagement of GITR induces Helios downregulation by CD4 Treg. FIG. 36A shows tumor growth in WT B6 mice inoculated s.c. with B16/F10 and anti-GITR or isotype Abs were injected on days 3, 6 and 9 (prophylactic treatment, left panel) or on days 10, 12, 14 and 16 (therapeutic treatment, right panel). FIG. 36B shows IFNγ production after in vitro restimulation with PMA and ionomycin fourteen days later in FoxP3⁺CD4 cells from spleens and tumors of tumor-bearing mice. FIG. 36C) shows IFNγ production in intratumoral CD8 Teff cells after in vitro restimulation with PMA and ionomycin. FIGS. 36D and 36E show recovery, Helios expression and anergic phenotype (FR4 and CD73) on day 21 in Treg from spleen cells from Rag2^{-/-} hosts that were transferred with Helios WT (FoxP3-Cre) or Helios KO (Helios^{fl/fl}.FoxP3-Cre) CD4 Treg. Rag2^{-/-} hosts were injected with anti-GITR (DTA-1) or isotype control Ab on days 0, 7, 14 and 20. FIG. 36F shows expression of effector cytokines (TNFα and IFNγ) after in vitro stimulation with PMA and ionomycin in spleen cells.

### DETAILED DESCRIPTION OF INVENTION

In some aspects, the disclosure relates to methods and compositions for modulating the differentiation of Treg cells. Regulatory T cells (Treg) are a sub-population of T cells that modulate the immune system and maintain tolerance to self-antigens (e.g., prevent autoimmune disease) via the suppression or down regulation of effector T cell (Teff) induction and proliferation. Generally, Treg cells are classified by expression of molecular markers, for example CD4 FoxP3⁺ Treg cells, or CD8⁺CD44 CD122⁺Ly49⁺ Treg cells. CD4+ Treg suppress a variety of effector T cell (Teff) functions (e.g., inflammatory responses). CD8+ Treg suppress development of autoantibody formation by inhibiting function of follicular T-helper cells. The T cell specific transcription factor (TF) Helios, is expressed by both CD4 and CD8 regulatory lineages.

Helios is a T cell-specific zinc finger transcription factor that is encoded by the Ikzf2 gene. It belongs to the Ikaros family of zinc finger proteins, which also includes Ikaros (Ikzf1), Aiolos (Ikzf3), Eos (Ikzf4), and Pegasus (Ikzf5). Helios, along with other Ikaros proteins, regulate lymphocyte development and differentiation. Until the present disclosure, however, the role of Helios deficiency on Treg activity in the face of altered immunological environments, including infection, inflammation and aging, had not been investigated.

### Induction of Treg Differentiation

The invention is based, at least in part, on the surprising discovery that the transcription factor Helios controls differentiation of CD4⁺ Treg and CD8⁺ Treg cells. In particular, the disclosure is based upon the recognition that inhibition of Helios activity and/or expression levels in CD4⁺ Treg and CD8⁺ Treg cells induces differentiation of the Treg cells into effector T cells (Teff) and/or removes the immunosuppressive phenotypes of the Treg cells. Thus, agents that modulate Helios activity and/or expression levels in Treg cells are useful for the treatment of certain diseases (e.g., cancers or autoimmune diseases) where therapeutic benefit could be derived from either enhancing or suppressing a subject's immune response.

Accordingly, one aspect of the disclosure provides a method for inducing differentiation of a regulatory CD4⁺ T (CD4⁺ Treg) cell to a CD4⁺ effector T cell, the method comprising contacting the CD4⁺ Treg with an agent that decreases Helios activity and/or Helios expression. Within the CD4⁺ T lymphocyte cell population, three categories of regulatory T cells have been described: TH3 cells, Type 1 regulatory (Tr1) cells, and CD4⁺CD25⁺ T regulatory cells (“Treg”). TH3 cells function via the secretion of TGF-β and can be generated in vitro by stimulation in the presence of IL-4 or in vivo through oral administration of low dose antigens (Chen et al., Science 265:1237-1240, 1994; Inobe et al., Eur. J. Immunol. 28:2780-2790, 1998). Type 1 regulatory T cells (Tr1) suppress T cells through the production of IL-10 and TGF-β and are derived by stimulation of memory T cells in the presence of IL-10 (Groux et al., Nature 389:737-742, 1996; Groux et al., J. Exp. Med. 184:19-29, 1996). CD4⁺CD25⁺ regulatory T cells are thought to function as a regulator of autoimmunity by suppressing the proliferation and/or cytokine production of CD4+CD25− T cell responder cells at the site of inflammation. CD25 is a transmembrane protein that functions as the alpha chain of the IL-2 receptor and is present on the surface of CD4⁺ Treg cells.

In some embodiments, the CD4⁺ Treg cell is FoxP3⁺CD25⁺. Forkhead box 3 protein (FoxP3) is well-established as the “master gene” that regulates the development and function of CD4⁺CD25⁺ Treg cells. Methods for the isolation of human Foxp3+ Treg cells are known. For instance, Hoffmann, P. et al. Biol Blood Marrow Transplant 12, 267-74 (2006) describe the isolation of CD4⁺CD25⁺ T cells with regulatory function from standard leukapheresis products by using a 2-step magnetic cell-separation protocol. The generated cell products contained on average 49.5% Foxp3⁺ Treg cells. Also, commercial kits, e.g. CD4⁺CD25⁺ Regulatory T Cell Isolation Kit from Miltenyi Biotec or Dynal® CD4⁺CD25⁺ Treg Kit from Invitrogen are also available.

As used herein the term “CD4 effector T cells” refers to a subset of T cells which are associated with cell-mediated immune response. They are characterized by the secretion of one or more effector cytokines such as, but not limited to, IFN-γ, TNF-α, IL-17, IL-2 and granzyme B.

As used herein, the term “differentiation of a regulatory CD4⁺ T (CD4⁺ Treg) cell to a CD4⁺ effector T cell” refers to the phenotypic conversion of a Treg cell to an effector T (Teff) cell. In some embodiments, the conversion of a CD4⁺ Treg cell to a CD4 effector cell is characterized by the expression of effector cytokines. Examples of effector cytokines expressed by Teff include tumor necrosis factor alpha (TNF-α), interferon-γ (IFN-γ), interleukin-17 (IL-17), interleukin-2 (IL-2), and Granzyme B. However, the skilled artisan appreciates that other effector cytokines may also be expressed by differentiated T effector cells.

An agent that decreases Helios activity and/or Helios expression can be a peptide, polypeptide, small molecule, antibody, or an RNAi molecule. For example, the agent can be an antibody that interferes with Helios activity or expression by binding to a target on the surface of a CD4⁺ Treg cell. Examples of targets include glucocorticoid-induced tumor necrosis factor receptor (GITR), CD134 (OX-40), CD47, CD137 (4-1BB), Neuropilin-1 (Nrp-1), and 5′-nucleotidase (CD73). Accordingly, in some embodiments, the agent is an anti-GITR, anti-OX-40, anti-CD47, anti-4-1BB, anti-Nrp-1, or anti-CD73 antibody. In some embodiments, the agent is a small molecule, e.g., a small molecule zinc finger protein inhibitor. Examples of small molecule zinc finger protein inhibitors include azodicarbonamide, C-nitroso compounds (e.g., 3-nitrosobenzamide (NOBA) and 6-nitroso-1,2-benzopyrone (NOBP)), 2,2′-di-thiobisbenzamide (DIBA), Pyridinioalkanoyl thiolesters (PATES; e.g., N-[2-(5-pyridiniovaleroylthio)benzoyl]sulfacetamide bromide), and Bis-Thiadizolbenzene-1,2-diamine. In some embodiments, the agent is a STAT5B inhibitor, such as, but not limited to, AG-490 ((E)-2-Cyano-3-(3,4-dihydrophenyl)-N-(phenylmethyl)-2-propenamide). In some embodiments, the agent is an RNAi molecule. Any suitable RNAi molecule (e.g., dsRNA, siRNA, shRNA, lcnRNA, and miRNA) can be used in the methods described in the instant disclosure. RNAi molecules can be modified (e.g., having modified nucleobases or backbones), or unmodified. For example, an RNAi molecule targeting the Ikzf2 gene can be used to silence expression of Helios. In some embodiments, the agent is a genome editing molecule (e.g., a TALEN molecule or CRISPR/Cas molecule) that decreases Helios activity and/or Helios expression, for example by silencing the Ikzf2 gene.

As used herein a “decrease in Helios activity and/or Helios expression” comprises any statistically significant decrease in the transcriptional activity and/or expression level (e.g., protein level or nucleic acid (mRNA or DNA)) within a Treg cell relative to an appropriate control. Such a decrease can include, for example, at least a 5%, 10%, 15%, 20%, 25%, 30%, 35%, 40%, 45%, 50%, 55%, 60%, 65%, 70%, 75%, 80%, 85%, 90%, 95%, or 100% decrease in the activity and/or expression of Helios within a Treg cell that has been contacted with an agent relative to a Treg cell that has not been contacted with an agent.

Helios activity and/or Helios expression can be measured or quantified by any suitable method in the art. For example, quantitative PCR, microarray analysis, northern blot, and southern blot are suitable methods for determining the mRNA or DNA level of Helios in a cell. Helios protein level can be measured directly by Western blot, or indirectly by methods such as flow cytometry. Activity of Helios can be measured directly or indirectly, for example by measuring the expression levels of genes known to be regulated by Helios such as, but not limited to BirC2, Bag2, NFAT5, Bcl-2, IL2Ra, Jak2, and Stat5b.

In some aspects, the present disclosure relates to the recognition that inhibition of Helios activity and/or expression level induces the differentiation of CD8⁺ T cells. Accordingly, the disclosure provides a method for inducing differentiation of a regulatory CD8+T (CD8⁺ Treg) cell to a CD8^{+/}PD1⁺/TIM3⁺ T cell, the method comprising contacting the regulatory T cell with an agent that decreases Helios activity and/or Helios expression.

CD8⁺ Treg cells are known in the art (see, for example, Hye-Jung Kim, et al. 2010. Inhibition of follicular T-helper cells by CD8+ regulatory cells is essential for self tolerance. Nature 467:328; Wang et al. Immunology and Cell Biology (2009) 87, 192-193). Programmed cell death-1 (PD-1) is a member of the CD28 superfamily that delivers negative signals upon interaction with its two ligands, PD-L1 or PD-L2 (see, for example, Jin et al. Curr Top Microbiol Immunol. 2011; 350:17-37). PD-1 and its ligands are broadly expressed and exert a wider range of immunoregulatory roles in T cells activation and tolerance. The PD-1 pathway is known to affect survival and/or proliferation of exhausted CD8 T cells (S. D. Blackburn, et al. Selective expansion of a subset of exhausted CD8 T cells by alphaPD-L1 blockade. Proc Natl Acad Sci USA 105, 15016 (Sep. 30, 2008); C. Petrovas et al., SIV-specific CD8+ T cells express high levels of PD1 and cytokines but have impaired proliferative capacity in acute and chronic SIVmac251 infection. Blood 110, 928 (Aug. 1, 2007)). TIM-3 is expressed by terminally-differentiated TH1 and TC1 cells and its engagement can trigger cell death (C. Zhu et al., The Tim-3 ligand galectin-9 negatively regulates T helper type 1 immunity. Nat Immunol 6, 1245 (December, 2005)). PD-1 and TIMP-3 are associated with T cell exhaustion and loss of cytolytic function (E. J. Wherry, T cell exhaustion. Nat Immunol 12, 492 (June, 2011); K. Sakuishi et al., Targeting Tim-3 and PD-1 pathways to reverse T cell exhaustion and restore anti-tumor immunity. J Exp Med 207, 2187 (Sep. 27, 2010)).

In some embodiments, the CD8⁺ Treg is positive for Killer cell immunoglobulin like receptor (Kir⁺). Killer cell immunoglobulin like receptors (KIR) represent the human homologue of murine Ly49 proteins, which are C-type lectins that bind host major histocompatibility complex (MHC) class I. Regulatory CD8⁺Kir⁺ T cells selectively suppress CD4⁺ follicular helper T cell (TFH) activity through recognition of class I MHC peptide Qa-1 (mouse homolog of human leukocyte antigen E (HLA-E)) expressed at the surface of TFH cells, and dampen autoantibody responses.

As used herein, the term “differentiation of a regulatory CD8⁺ T (CD8⁺ Treg) cell to a CD8⁺/PD1⁺/TIM3⁺ T cell” refers to the phenotypic conversion of a CD8⁺ Treg cell to a CD8⁺ T cell having an exhausted phenotype, characterized by the expression of PD-1 and TIMP-3. CD8⁺ T cells expressing PD-1⁺ and Tim-3 do not produce IFN-γ, TNF-α, and IL-2 in response to PD-1 ligand (PDL1) and Tim-3 ligand, and exhibit a transcriptional state that is distinct from that of functional effector or memory T cells.

In some embodiments, CD8⁺/PD1⁺/TIM3⁺ T cells are characterized by increased expression of BLIMP-1. BLIMP-1 (also known as PR domain zinc finger protein 1) is known to regulate the terminal differentiation of diverse cell types (K. Hayashi, S. M. de Sousa Lopes, M. A. Surani, Germ cell specification in mice. Science 316, 394 (Apr. 20, 2007); V. Horsley et al., Blimp1 defines a progenitor population that governs cellular input to the sebaceous gland. Cell 126, 597 (Aug. 11, 2006); S. Roy, T. Ng, Blimp-1 specifies neural crest and sensory neuron progenitors in the zebrafish embryo. Current biology: CB 14, 1772 (Oct. 5, 2004); Y. Ohinata et al., Blimp1 is a critical determinant of the germ cell lineage in mice. Nature 436, 207 (Jul. 14, 2005)) and can promote terminal differentiation of CD8 cells at the expense of their potential to remain in the memory pool (H. Shin et al., A role for the transcriptional repressor Blimp-1 in CD8(+) T cell exhaustion during chronic viral infection. Immunity 31, 309 (Aug. 21, 2009); R. L. Rutishauser et al., Transcriptional repressor Blimp-1 promotes CD8(+) T cell terminal differentiation and represses the acquisition of central memory T cell properties. Immunity 31, 296 (Aug. 21, 2009)).

In some embodiments, the CD8⁺/PD1⁺/TIM3⁺ T cells express anti-inflammatory cytokines, such as IL-10.

An agent that induces differentiation of CD8⁺ Treg by decreasing Helios activity and/or Helios expression can be a peptide, polypeptide, small molecule, antibody, or an RNAi molecule. In some embodiments, the agent is an anti-Kir or anti-Ly49F antibody. The antibody can also be a bispecific antibody, for example a bispecific anti-CD8/anti-Kir antibody. In some embodiments, the agent is a small molecule, such as a zinc finger protein inhibitor or a Stat5B inhibitor, such as, but not limited to, AG-490 ((E)-2-Cyano-3-(3,4-dihydrophenyl)-N-(phenylmethyl)-2-propenamide). Signal transducer and activator of transcription 5B (Stat5B) is a transcription factor that regulates a variety of cellular processes including apoptosis, TCR signaling, and Treg lineage commitment.

Tregs are known to play a role in suppressing effective Th1 responses in tumors. Without wishing to be bound by any particular theory, decreasing the activity level and/or expression level of Helios induces differentiation of CD4⁺ and/or CD8⁺ Treg into effector T cells, thereby removing the suppressive phenotypes of these T cells. Therefore, methods and compositions described by the disclosure may be useful in the treatment of diseases such as, but not limited to, cancer and infections. In some aspects, the disclosure provides a method for treating cancer in a subject, the method comprising administering to the subject an agent that induces differentiation of regulatory CD4⁺ T (CD4⁺ Treg) cells to CD4⁺ effector cells by decreasing Helios activity and/or Helios expression. In some aspects, the disclosure provides a method for treating cancer in a subject, the method comprising administering to the subject an agent that induces differentiation of regulatory CD8⁺ T (CD8⁺ Treg) cells to CD8⁺/PD1⁺/TIM3⁺ T cells by decreasing Helios activity and/or Helios expression.

A subject is a patient having or suspected of having a disease (e.g., cancer or an infection). A subject can be a human, non-human primate, rodent, dog, cat, horse, pig, or fish. In some embodiments, a subject is a patient having or suspected of having cancer. In some embodiments, a subject is a patient having or suspected of having an infection.

Examples of cancers that can be treated using the methods and compositions described by the disclosure include, but are not limited to, brain cancer, breast cancer, bladder cancer, pancreatic cancer, prostate cancer, liver cancer, kidney cancer, lymphoma, leukemia, lung cancer, colon cancer ovarian cancer, gastric cancer, cervical cancer, gliomas, head and neck cancers, esophagus cancer, gall bladder cancer, thyroid cancer, and melanoma.

Agents that induce the differentiation of CD4+ and/or CD8+ Treg cells are described elsewhere in the disclosure and include peptides, polypeptides, small molecules, antibodies, or RNAi molecules. Examples of agents that induce differentiation of CD4+ Treg cells include antibodies targeting GITR, OX-40, CD47, 4-1BB, Nrp-1, and CD73. Examples of agents that induce differentiation of CD8+ Treg cells include anti-Kir, anti-Ly49, or bispecific anti-CD8/anti-Kir antibodies. In some embodiments, the agent is a small molecule, for example a zinc finger protein inhibitor, or a Stat5B inhibitor.

In some embodiments of any one of the methods provided herein, the method further comprises contacting a cell with an agent that activates T effector cells. In some embodiments of any one of the methods provided herein, the method further comprises administering to the subject an agent that activates T effector cells. In some embodiments, an agent that activates T effector cells is an inhibitor of an immune checkpoint protein. Examples of immune checkpoint proteins include inhibitory receptors and their cognate ligands. Examples of inhibitory receptors include, but are not limited to, Cytotoxic T-cell-Lymphocyte-associated Antigen 4 (CTLA4), Programmed Cell Death protein 1 (PD1), Lymphocyte Activation Gene 3 (LAG3), T-cell Membrane Protein 3 (TIM-3), 4-1BB (CD137), and T cell Ig and ITIM domain (TIGIT). Examples of immune checkpoint proteins that are ligands include, but are not limited to, PD1 Ligands 1 and 2 (PDL-1, PDL-2), B7-H3, B7-H4, and 4-1BB (CD137) ligand. In some embodiments, the immune checkpoint inhibitor is an inhibitor of an immune checkpoint protein selected from the group consisting of: PD-1, TIM-3, and TIGIT.

PD-1 expression on T cells is induced upon T cell activation (see, for example, Baumeister, S. H., Freeman, G. J., Dranoff, G., and Sharpe, A. H. 2016. Coinhibitory Pathways in Immunotherapy for Cancer. Annu Rev Immunol 34:539-573). Without wishing to be bound by theory, when T cells are repetitively stimulated by antigen, the level of PD-1 remains high, T cells undergo epigenetic modifications and changes in transcription factor expression, leading to a dysfunctional T cell state. In some embodiments, an agent that activates T effector cells, e.g., an immune checkpoint inhibitor, is a PD-1 antagonist.

TIM-3 promotes T cell tolerance. TIM-3 and PD-1 are co-expressed by dysfunctional CD8 T cells. (see, for example, Ngiow, S. F., von Scheidt, B., Akiba, H., Yagita, H., Teng, M. W., and Smyth, M. J. 2011. Cancer Res 71:3540-3551). In some embodiments, an agent that activates T effector cells, e.g., an immune checkpoint inhibitor, is a TIM-3 antagonist.

TIGIT is expressed on tumor infiltrating CD8 T cells and is coexpressed with PD-1. (see, for example, Chauvin, J. M., et al. 2015. J Clin Invest 125:2046-2058). In some embodiments, an agent that activates T effector cells, e.g., an immune checkpoint inhibitor, is a TIGIT antagonist.

An immune checkpoint inhibitor can be a peptide, antibody, interfering RNA, or small molecule. Generally, immune checkpoints are initiated by ligand-receptor interactions between immune checkpoint proteins. See, for example, Pardoll et al., Nature Reviews Cancer, 12: 252-264, 2012. In some embodiments, such interactions are blocked by using specific antibodies (e.g., antibodies that bind specifically to an immune checkpoint protein or its interacting partner), recombinant protein ligands, and/or soluble recombinant receptor proteins. Thus, in some embodiments, the immune checkpoint inhibitor is an antibody (e.g., a monoclonal antibody), or an Ig fusion protein.

Methods of producing antibodies are well known in the art. For example, an epitope of a target protein (e.g., an immune checkpoint protein) can be used to generate polyclonal antibodies in animals. Alternatively, a monoclonal antibody can be produced. Methods of producing monoclonal and polyclonal antibodies are described, for example, in Antibodies: A Laboratory Manual, Harlow and Lane, Cold Spring Harbor Laboratory, New York, 1988. Non-limiting examples of antibody immune checkpoint inhibitors include Ipilimumab, Tremelimumab, MDX-1106 (BMS-936558), MK3475, CT-011 (Pidilizumab), MDX-1105, MPDL3280A, MEDI4736, and MGA271. In some embodiments, the immune checkpoint inhibitor is selected from the group consisting of: anti-PD-1 antibody, anti-TIM-3 antibody, and anti-TIGIT antibody.

In some embodiments, the method of treating cancer further comprises administering to the subject a chemotherapeutic agent. Generally, chemotherapeutic agents are classified by their mode of action and/or chemical structure. Examples of chemotherapeutic drug classes include alkylating agents (e.g., nitrogen mustards such as cyclophosphamide, nitrosureas, alkyl sulfonates, triazines, ethlyenimines), platinum drugs (e.g., cisplatin, carboplatin, oxalaplatin), antimetabolites (e.g., 5-fluorouracil (5-FU), 6-mercaptopurine (6-MP), capecitabine, cytarabine, floxuridine, fludarabine, gemcitabine, hydroxyurea, methotrexate, pemetrexed), anti-tumor antibiotics (e.g., daunorubicin, doxorubicin, epirubicin, idarubicin, actinomycin-D, bleomycin, mitomycin-C, mitoxantrone), topoisomerase inhibitors (e.g., topotecan, irinotecan (CPT-11), etoposide (VP-16), teniposide), mitotic inhibitors (e.g., paclitaxel, docetaxel, ixabepilone, vinblastine, vincristine, vinorelbine, estramustine), corticosteroids (e.g., prednisone, methylprednisone, dexamethasone), and monoclonal antibodies (e.g., gemtuzumab, brentuximab, trastuzumab, bevacizumab, cetuximab, rituximab).

### Suppression of Treg Differentiation

In some cases, it may be desirable to inhibit or suppress the differentiation of Treg cells. For example in the context of autoimmune disease, it may be beneficial to stabilize suppressive Treg phenotypes by preventing differentiation of Treg cells into Teff cells. Therefore, in some aspects the disclosure provides a method for inhibiting differentiation of a CD4⁺ regulatory T cell to a CD4⁺ effector T cell, the method comprising contacting the CD4⁺ regulatory T cell with an agent that increases Helios activity and/or Helios expression. In some aspects, the disclosure relates to a method for inhibiting differentiation of a CD8⁺ regulatory T cell to a CD8⁺/PD1⁺/TIM3⁺ T cell, the method comprising contacting the CD8⁺ regulatory T cell with an agent that increases Helios activity and/or Helios expression.

Compositions and methods for inhibiting differentiation of Treg cells into effector T cells are useful in the treatment of autoimmune disease. Without wishing to be bound by any particular theory, inhibition of differentiation by increasing Helios activity and/or Helios expression level stabilizes the suppressive phenotype of Treg cells and dampens the immune response to self-antigens.

Accordingly, in some aspects, the disclosure provides a method for treating autoimmune disease in a subject, the method comprising administering to the subject an agent that inhibits differentiation of CD4⁺ regulatory T cells to CD4⁺ effector cells, by increasing Helios activity and/or Helios expression. In some aspects, the disclosure provides a method for treating autoimmune disease in a subject, the method comprising administering to the subject an agent that inhibits differentiation of CD8⁺ regulatory T cells to CD8⁺/PD1⁺/TIM3⁺ T cells, wherein the agent increases Helios activity and/or Helios expression.

Agents that inhibit differentiation of Treg cells into effector T cells or exhausted T cells can be peptides, polypeptides, small molecules, antibodies, or RNAi molecules. In some embodiments, agents that inhibit differentiation of Treg cells into effector T cells or exhausted T cells include, but are not limited to, inflammatory cytokines, such as IL-12 and IL-18.

Examples of autoimmune diseases that can be treated with the methods and compositions described herein include alopecia areata, autoimmune hemolytic anemia, autoimmune hepatitis, dermatomyositis, diabetes (type 1), juvenile idiopathic arthritis, glomerulonephritis, Graves' disease, Guillain-Barré syndrome, idiopathic thrombocytopenic purpura, myasthenia gravis, myocarditis, multiple sclerosis (MS), pemphigus/pemphigoid, pernicious anemia, polyarteritis nodosa, polymyositis, primary biliary cirrhosis, psoriasis, rheumatoid arthritis (RA), scleroderma/systemic sclerosis, Sjögren's syndrome (SjS), systemic lupus erythematosus (SLE), thyroiditis, uveitis, vitiligo, and granulomatosis with polyangiitis (Wegener's granulomatosis).

### Administration

The agent that decreases the activity and/or expression level of Helios is administered in an amount effective to stimulate an immune response to the antigen in the subject. The term “effective amount” as provided herein, refer to a sufficient amount of the agent to provide an immunological response and corresponding therapeutic effect. The exact amount required will vary from subject to subject, depending on the species, age, and general condition of the subject, the severity of the condition being treated, and the particular agent, mode of administration, and the like. An appropriate “effective” amount in any individual case may be determined by one of ordinary skill in the art using routine experimentation.

Agents described by the disclosure are administered to a subject by any suitable route. For example, agents can be administered orally, including sublingually, rectally, parenterally, intracisternally, intravaginally, intraperitoneally, topically and transdermally (as by powders, ointments, or drops), bucally, or nasally. In some embodiments, an agent described by the disclosure is part of a pharmaceutical composition or pharmaceutical preparation. Pharmaceutical compositions or pharmaceutical preparations of the disclosure may include or be diluted into a pharmaceutically-acceptable carrier. The term “pharmaceutically-acceptable carrier” as used herein means one or more compatible fillers, diluants or other such substances, which are suitable for administration to a human or other mammal such as a dog, cat, or horse. The term “carrier” denotes an organic or inorganic ingredient, natural or synthetic, with which the active ingredient is combined to facilitate the application. The carriers are capable of being commingled with the preparations of the present invention, and with each other, in a manner such that there is no interaction which would substantially impair the desired pharmaceutical efficacy or stability. Carriers suitable for oral, subcutaneous, intravenous, intramuscular, etc. formulations can be found in Remington's Pharmaceutical Sciences, Mack Publishing Company, Easton, Pa.

The exact amount of an agent that decreases the activity and/or expression level of Helios, e.g., and optionally an immune modulator, required to achieve an effective amount will vary from subject to subject, depending, for example, on species, age, and general condition of a subject, severity of the side effects or disorder, identity of the particular agent that decreases the activity and/or expression level of Helios, identity of the particular immune checkpoint inhibitor, mode of administration, and the like. An effective amount may be included in a single dose or multiple doses.

In some embodiments, in a combination therapy with an agent that decreases the activity and/or expression level of Helios and an immune modulator, each dose is a combination of the agent that decreases the activity and/or expression level of Helios and the immune modulator. In some embodiments, the combination of the agent that decreases the activity and/or expression level of Helios and the immune modulator is administered as a single composition (e.g., a heterogeneous mixture of the two inhibitors). In some embodiments, the agent that decreases the activity and/or expression level of Helios and the immune modulator may be independently administered (e.g., individually administered as separate compositions) at the same time or administered separately at different times in any order. For example, an agent that decreases the activity and/or expression level of Helios can be administered prior to, concurrently with, or after administration of an immune modulator.

In certain embodiments, the duration between an administration of the agent that decreases the activity and/or expression level of Helios and an administration of the immune modulator is about one hour, about two hours, about six hours, about twelve hours, about one day, about two days, about four days, or about one week, wherein the administration of the agent that decreases the activity and/or expression level of Helios and the administration of the immune modulator are consecutive administrations. In some embodiments, an administration of an agent that decreases the activity and/or expression level of Helios is occurs at least 24 hours (1 day), 2 days, 3 days, or 4 days prior to the administration of an immune modulator.

An effective amount of a compound (e.g., an agent that decreases the activity and/or expression level of Helios or an immune checkpoint inhibitor) may vary from about 0.001 mg/kg to about 1000 mg/kg in one or more dose administrations, for one or several days (depending on the mode of administration). In certain embodiments, the effective amount varies from about 0.001 mg/kg to about 1000 mg/kg, from about 0.01 mg/kg to about 750 mg/kg, from about 0.1 mg/kg to about 500 mg/kg, from about 1.0 mg/kg to about 250 mg/kg, from about 1.0 mg/kg to about 10.0 mg/kg, or from about 10.0 mg/kg to about 150 mg/kg. In some embodiments, an effective amount of a compound (e.g., an agent that decreases the activity and/or expression level of Helios or an immune checkpoint inhibitor) is from 1.0 mg/kg to 10.0 mg/kg, e.g., 1 mg/kg, 2 mg/kg, 3 mg/kg, 4 mg/kg, 5 mg/kg, 6 mg/kg, 7 mg/kg, 8 mg/kg, 9 mg/kg, or 10 mg/kg.

### Screening Methods

The disclosure relates, in part, to methods of identifying agents that modulate the differentiation of Treg cells to effector T cells or exhausted T cells. In some embodiments, the agents modulate the activity and/or expression levels of Helios. Modulation can be an increase or a decrease of Helios activity level and/or Helios expression level.

In some aspects, the disclosure provides a method for identifying candidate compounds for modulating Helios activity and/or Helios expression, the method comprising: contacting a regulatory T cell with a test compound; measuring Helios activity level and/or Helios expression level in the cell; and, identifying the test compound as a candidate compound for modulating Helios activity and/or Helios expression if the Helios activity level and/or Helios expression level is increased or decreased relative to a control cell that has been treated with a compound known to not modulate Helios activity level and/or Helios expression level.

The measurement of Helios activity level and/or expression level can be performed by any suitable method in the art. For example, quantitative PCR, microarray analysis, northern blot, and southern blot are suitable methods for determining the RNA or DNA level of Helios in a cell. Helios protein level can be measured directly by Western blot, or indirectly by methods such as flow cytometry. Activity of Helios can be measured directly or indirectly, for example by measuring the transcription levels of genes known to be regulated by Helios, such as but not limited to BirC2, Bag2, NFAT5, Bcl-2, IL2Ra, Jak2 and STAT5b. In some embodiments, the method further comprises measuring FoxP3 activity level and/or FoxP3 expression level. The measurement of FoxP3 activity level and/or FoxP3 expression level can be performed by any suitable method known in the art such as, but not limited to quantitative PCR, microarray analysis, northern blot, southern blot and Western blot.

In some embodiments, the regulatory T cell is a FoxP3+CD25+CD4+ T cell, or a Kir+ CD8+ T cell.

As used herein, a “test compound” can be any chemical compound, for example, a peptide, polypeptide, antibody, small molecule, RNAi molecule and CRISPR/Cas molecule.

In some embodiments, measuring of Helios activity level and/or expression level is performed by a protein-based screening method. A “protein-based screening method” refers to any diagnostic assay that measures the interaction between a protein and a target (e.g., a protein, peptide, nucleic acid, or metabolite). Examples of protein-based screening methods include western blot, affinity chromatography and fluorescence-assisted cell sorting (FACS). For example, a protein-based screening method may comprise the steps: contacting the cell with a detectable antibody targeting Helios; contacting the cell with a detectable antibody targeting FoxP3; contacting the cell with at least one detectable antibody targeting an effector cytokine; and, detecting the level of the detectable antibodies. Examples of antibodies that target a cytokine include antibodies directed to TNF-α, IFN-γ, IL-17, IL-10, and IL-2. Detectable antibodies can be detected by spectroscopic, photochemical, biochemical, immunochemical, chemical, or other physical means. For example, detectable antibodies can be fluorescently labeled, radiolabeled, or chemilumescently labeled (e.g., horseradish peroxidase, HRP).

The present invention is further illustrated by the following Example, which in no way should be construed as further limiting. The entire contents of all of the references (including literature references, issued patents, published patent applications, and co pending patent applications) cited throughout this application are hereby expressly incorporated by reference.

### EXAMPLES

### Example 1: Materials and Methods

### Mice.

C57BL/6J (B6), FoxP3.GFP transgenic, FoxP3^{YFP}-Cre, OT-II transgenic, RIP-mOVA transgenic, CD4^{-/-} and CD8^{-/-} mice were used for this study. Rag2^{-/-}, Rag2^{-/-}γc^{-/-}, Rag2^{-/-}Prf^{-/-}, K^{b-/-}D^{b-/-} and CD45.1⁺ C57BL/6 were from Taconic Farms. Helios-, Heliosfl/fl, Heliosfl/fl/CD4-Cre, and HY-TCR KI mice have been described. Helios^{fl}/FoxP3^{YFP}-Cre mice were generated by crossing Helios^{fl} mice to FoxP3^{YFP}-Cre mice. Helios^{-/-} mice and Helios^{+/+} littermate controls were used throughout the experiments described here.

### Antibodies, Flow Cytometry.

Fluorescence dye labeled Abs specific for CD3 (145-2C11), CD4 (L3T4), CD8 (53-6.7), TCR (H57-597), B220 (RA3-6B2), CD44 (IM7), CD62L (MEL-14), Fas (15A7), IgM (11/41), PD-1 (J43), CXCR5 (2G8), CD122 (TM-β1), Ly49 (14B11), CD69 (H1.2F3), CD11b (M1/70), Gr-1 (RB6-8C5), NKG2D (CX5), CD45.1 (A20), HY-TCR (T3.70), V135 (MR9-4), CD45.1 (A20), Helios (22F6), FoxP3 (NRRF-30), RORγt (Q31-378), Lag3 (C9B7W), TIM-3 (RMT3-23), CD127 (A7R34), p-STAT5 (D47E7) were purchased from BD, eBioscience, Biolegend and Cell Signaling. Intracellular staining for Helios, FoxP3 and RORγt was performed using the FoxP3 staining buffer set (eBioscience). Intracellular staining for active Caspase-3 and was done using rabbit mAb (5A1E) followed by labeling with anti-rabbit AlexaFlour 647 Ab from Cell Signaling Technology after fixation of cells with Cytofix/Cytoperm buffer (BD Bioscience). Intracellular detection of IFN-γ (XMG1.2), IL-17 (TC11-18H10), IL-4 (11B11), TNFα(MP6-XT22) and IL-10 (JES5-16E3) was performed after restimulation of cells with 50 ng ml⁻¹ phorbol-12-myristate 13-acetate (Sigma) and 500 ng ml⁻¹ ionomycin (Sigma) and Golgistop (BD bioscience) for 4 hours. Stimulated cells were stained for surface markers first and fixed and permeabilized and then stained with mAbs for different cytokines.

For the detection of p-STAT5, single-cell suspensions were prepared from spleens isolated from Helios^{+/+}, Helios^{-/-}, Helios^{fl/fl}/CD4-Cre or Helios^{fl/fl}/FoxP3-Cre bone marrow chimeric mice. Five million splenocytes per condition were pre-treated with antibodies that block Fc receptors and were then stained for surface antigens CD8a, TCR-beta and CD25. These cell suspensions were washed in DMEM and subsequently serum-starved in 250 al of DMEM for 45 min at 37° C. The cells remained unstimulated or stimulated with 100 U/ml IL-2 (eBiosciences) for 60 min and washed with PBS. The cells were fixed, permeabilized and stained for intercellular FoxP3 (eBiosciences) and p-STAT5 (Cell Signaling) according to the flow cytometry protocol from Cell Signaling Technology.

### Gene Expression Profiling.

CD8 T cells were enriched from spleen cells of WT B6 mice using CD8 microbeads (Miltenyi) and stained with CD3, CD8, CD122 and Ly49. Ly49⁺ and L49⁻CD8 cells were purified by cell sorting. RNA was prepared with the RNeasy mini kit according to manufacturer's instructions (Qiagen). RNA amplification, labeling and hybridization to MOA430 2.0 chips (Affymetrix) were done at the Core Facility of Dana Farber Cancer Institute.

### ChiP-Seq Analysis.

Purified CD4 (CD3⁺CD4⁺CD25⁺) and CD8 Treg (CD3⁺CD8⁺Ly49⁺) from spleen of WT B6 mice were activated in vitro by incubating with microbeads coated with anti-CD3 and anti-CD28 Ab in supplement with IL-2 (CD4 Treg, 50 ng/ml) or IL-15 (CD8 Treg, 20 ng/ml) for 5 days. Activated CD4 and CD8 Treg were fixed for 10 min at 37° C. with 1% formaldehyde. The cells were washed twice in ice-cold PBS and the cell pellets were flash-frozen and stored at −80° C. For each Helios ChiP analysis in CD4 and CD8 Treg, 15×10⁶-20×10⁶ cells were used, and for each ChiP analysis of chromatin modification (trimethylation of histone H3 at Lys27 and acetylation of H3 at Lys27) 1.5×10⁶ cells were used. The following antibodies were used for the ChIP: anti-Helios (M-20, Santa Cruz), anti-H3K27me3 (07-449, Millipore), anti-H3K27ac (ab4729, Abcam), and normal rabbit immunoglobulin G (Control immunolglobulin G: 10500C, Life Technologies). Preparation of ChiP immunocomplexes and DNA fragments, assays for high-throughput sequencing and ChiP-seq informatics were performed.

### Hematopoietic Reconstitution.

For generation of bone marrow chimeras, Rag2^{-/-} hosts were treated lethal dose of radiation (900 rads) one day before BM cell transfer or irradiated two doses of 450 rads 4 hrs apart followed by reconstitution of BM cells. Bone marrow cells from donor mice were harvested by depleting NK1.1⁺, CD4⁺, CD8⁺ and TCR⁺ cells and 5×10⁶ cells were transferred intravenously. For generation of Helios sufficient and deficient BM chimeras, BM cells from Helios^{+/+}, Helios^{-/-}, FoxP3^{YFP}-Cre, Helios^{fl/fl}/Helios^{YFP}-Cre mice were transferred. For the generation of mixed BM chimera mice, 1:1 mixture of BM cells from CD4^{-/-} and Helios^{-/-}, from CD8^{-/-} and Helios^{-/-}, Helios+/+ and Helios−/−, Helios+/+ and Scurfy, Heliosfl/fl/CD4-Cre and Scurfy mice were injected, respectively. For the analysis of negative selection and development of FoxP3⁺CD4 Treg by self-reactive CD4 cells, WT B6 or RIP-mOVA Tg mice were reconstituted with BM cells from Helios^{+/+} or Helios^{-/-} OT-II Tg mice.

### Immunohistochemistry.

To assess immunopathology in multiple organs, mice were fixed with Bouin's solution before tissue sections were generated from paraffin-imbedded tissues and stained with hematoxylin and eosin. For the analysis of IgG deposition in kidney, 7 m acetone fixed frozen sections from kidney were stained with Alexa-Fluor® 488 conjugated anti-mouse IgG antibodies (Invitrogen). More than 10 tissue sections were examined for each experimental condition to verify the reproducibility. Quantification of positively stained areas was performed by using ImageJ software and is depicted as pixel²/area.

### LCMV-Armstrong Infection.

2 month or 6 month old Helios WT or KO mice were infected i.p. with 2×10⁵ PFU LCMV-Armstrong. At day 30, mice were sacrificed and analyzed for the lymphocyte profile using spleen cells. Kidneys were harvested and cryosections were generated for the analysis of IgG deposition.

### Assessment of CD8 Treg Suppressive Activity in Adoptive Hosts.

2×10⁶ B cells and 1×10⁶ CD25 depleted CD4 cells were transferred into Rag2^{-/-} hosts along with 5×10⁵ CD8 cells. For the preparation of CD8 cells, spleen cells were harvested from mice that were immunized with 100 μg KLH in CFA and FACS-sorted by labeling with Abs for CD3, CD8, CD122 and Ly49. Rag2^{-/-} hosts were immunized with NP₁₉-KLH in CFA at day 0 and reimmunized with NP₁₉-KLH in IFA at day 10. Serum was prepared at day 10 and at 15 for the measurement of primary and secondary responses, respectively.

### Transfer Model of Colitis.

Naïve CD4 cells (CD3⁺CD4⁺CD44^{lo}CD62L^{hi}, 4×10⁵) sorted from Helios WT mice were transferred into Rag2^{-/-} hosts alone or in combination with Helios WT or Helios KO CD4 Treg (CD3⁺CD4⁺CD25⁺, 1×10⁵). Three different Helios KO mice (Helios^{-/-}, Helios^{fl/fl}/CD4-Cre, Helios^{fl/fl}/FoxP3^{YFP}-Cre) were tested. For the experiment with CD4 Treg from Helios^{fl/fl}/CD4-Cre Mice, Treg cells were transferred into Rag2^{-/-} hosts when the mice showed ˜10% weight loss from the original weight when naïve CD4 T cells were transferred. Mice were weighed weekly and monitored for the signs of wasting disease. 6 wks after reconstitution, mice were sacrificed and spleen cells were analyzed for the immune cell profiling and intestines were analyzed for the histopathology.

CFSE Labeling and Transfer into K^{b-/-}D^{b-/-} Mice.

CD122⁺Ly49⁺, CD122⁺Ly49 and CD122 CD8 cells were FACS-sorted from WT B6 spleen cells. Cells were labeled with CFSE and transferred into K^{b-/-}/D^{b-/-} mice that were depleted of NK1.1⁺ cells and sub-lethally (600 rads) irradiated 24 hrs before cell transfer. 5 days after cell transfer, proliferation of CD8 cells was analyzed by measuring CFSE labeling intensity.

### ELISA for Antibodies.

For the detection of NP specific antibodies, ELISA plates were coated with 0.5 μg/ml NP₄—BSA or 1 μg/ml NP₂₃-BSA for the detection of high-affinity or total NP-specific antibodies (Biosearch Technologies). Serum harvested 14 days after immunization with NP₁₉-KLH in CFA and reimmunization with NP₁₉-KLH in IFA was used as a standard. 1:4000 dilution of this immune serum was defined as 100 units/ml. Antibodies with IgG isotype were detected by incubating plates with biotinylated anti-mouse IgG followed by streptavidin-peroxidase. The amounts of ANA, anti-dsDNA, anti-SS/A, anti-SS/B were determined with ELISA kits from Alpha Diagnostic International. For the detection of anti-thyroglobulin and anti-insulin Abs, porcine thyroglobulin (Sigma) and porcine insulin (Sigma) were used.

### Quantitative PCR.

Cells were enriched for CD8 cells using mouse CD8α microbeads (Miltenyi Biotech) and sorted on a FACSAria (BD Bioscience). RNA was extracted using RNeasy Plus micro kit (Qiagen). cDNA was generated using iScript cDNA Synthesis Kit (Bio-Rad). Relative quantification real time PCR was performed with the following primers: Blimp-1 forward: 5′-gacgggggtacttctgttca-3′ (SEQ ID NO: 1), Blimp-1 reverse: 5′-ggcattcttgggaactgtgt-3′ (SEQ ID NO: 2), GAPDH forward: ggagaaacctgccaagtatg-3′ (SEQ ID NO:3), GAPDH reverse: tgggagttgctgttgaagtc-3′ (SEQ ID NO:4). GAPDH was used an endogenous control.

### Statistical Analyses.

For calculation of statistical significance of the differences, Wilcoxon-Mann-Whitney rank sum test was performed for comparison of two conditions and the Kruskal-Wallis test was performed for comparison of more than two conditions. A P value <0.05 was considered to be statistically significant (*=<0.05, **=<0.01, ***=<0.001).

### Example 2: Helios is Expressed by CD4+ and CD8+ Treg

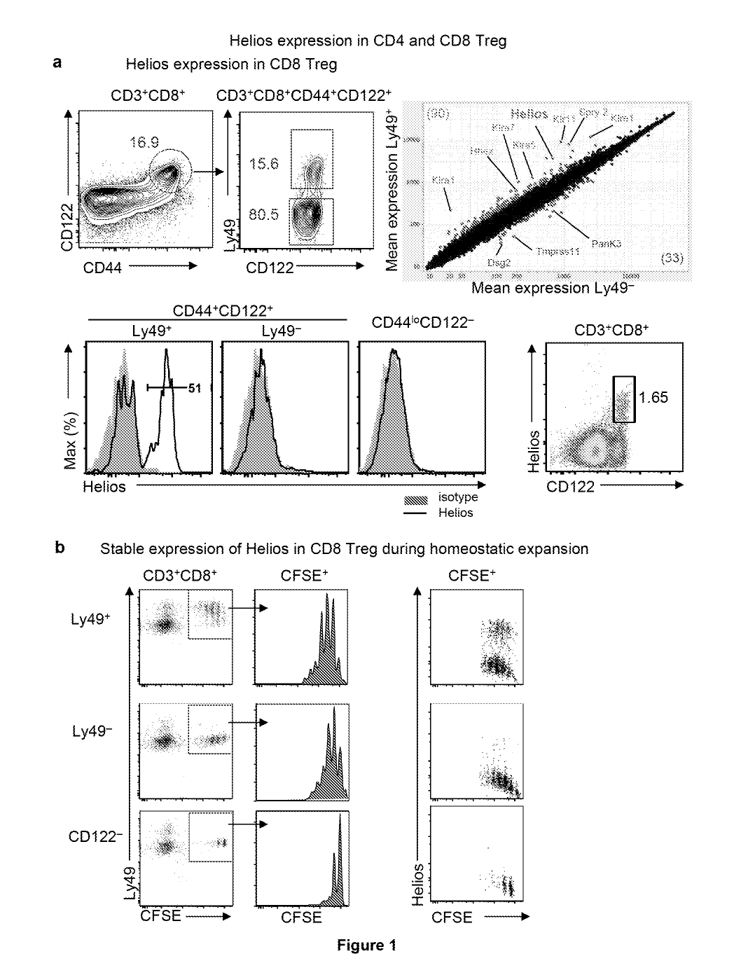

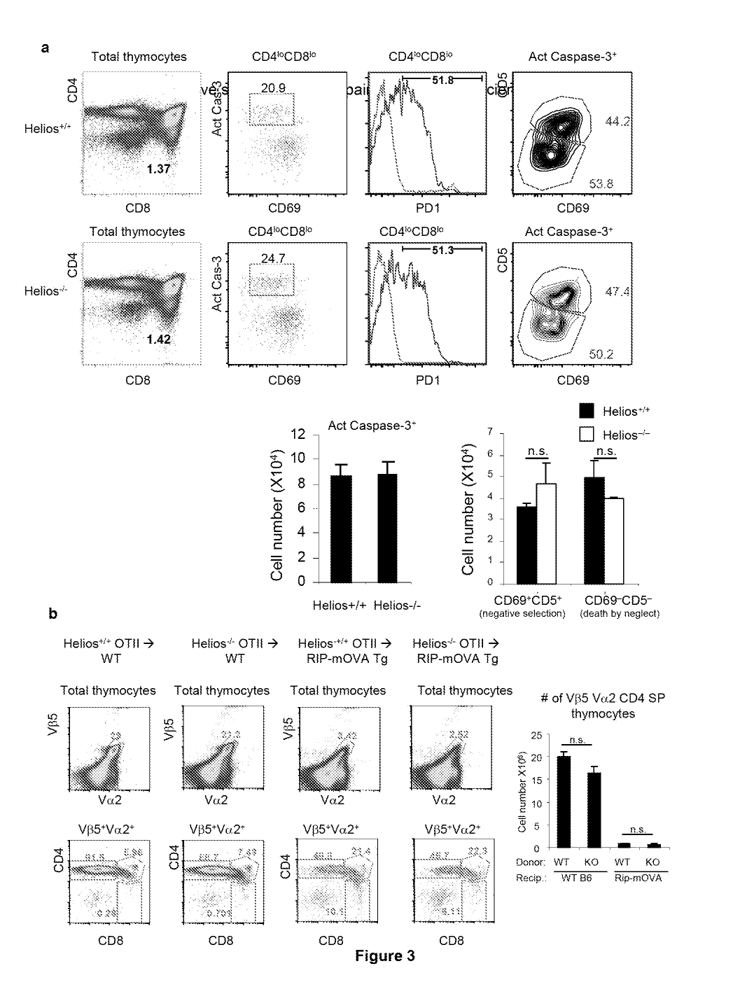

A subset of IL-15 dependent CD8 T cells that expresses the cell surface triad of CD44, CD122 and Ly49 (termed Ly49⁺CD8 cells) that represents less than 5% of CD8 cells accounts for Qa-1-restricted regulatory activity (FIG. 6). To gain insight into the genetic program that dictates CD8 Treg inhibitory activity, a DNA microarray analysis that compared gene expression by highly purified (>99%) CD8 Treg (CD44⁺CD122⁺Ly49⁺⁾ to genes expressed by conventional (CD44⁺CD122⁺Ly49⁻) CD8 cells was performed. Results indicate that Helios TF is expressed exclusively by Ly49⁺CD8 cells but not by conventional CD8 T cells (FIG. 1A). Analysis of Helios protein expression revealed that approximately 50% of Ly49⁺CD8 cells express Helios, while this TF is not expressed at detectable levels by Ly49⁻ CD8 cells (FIG. 1A, lower panel).

To determine whether Helios expression by CD8⁺ cells is stable, sorted CD44⁺CD122⁺Ly49⁺, CD44⁺CD122⁺Ly49⁻ as well as CD44⁻CD122⁻Ly49⁻ CD8 cells were transferred into sub-lethally irradiated NK-depleted K^{b-/-}D^{b-/-} mice and proliferation was monitored using CFSE. CFSE negative cells are of host origin. Both CD122⁺Ly49⁺ and CD122⁺Ly49⁻ CD8 cells underwent proliferation under these conditions (5-6 divisions) while CD122⁻Ly49⁻ CD8 cells underwent minimal proliferation (<2 divisions). Helios was stably expressed by Ly49⁺CD8 cells, while Ly49⁻ CD8 cells did not acquire expression during homeostatic proliferation in these lymphopenic hosts (FIG. 1B). Helios expression by these two Ly49⁺ and Ly49⁻ CD8 subsets was also stable after in vitro expansion in cultures supplemented with IL-15, an essential cytokine for survival and expansion of CD8 Treg, at all concentrations tested. These data suggest that the Helios TF is stably and selectively expressed by Ly49⁺CD8⁺ T cells and is associated with the specialized regulatory activity of this CD8 subset. Since Helios is also expressed by FoxP3⁺CD4⁺ Treg, the question of whether Helios⁺Ly49⁺CD8⁺ T cells also expressed FoxP3 was investigated. They did not, suggesting that the FoxP3-associated genetic program that operates in CD4⁺ Treg may be distinct from that of CD8⁺ Treg (FIG. 1C).

### Example 3: Development of Autoimmunity by Helios Deficient Mice

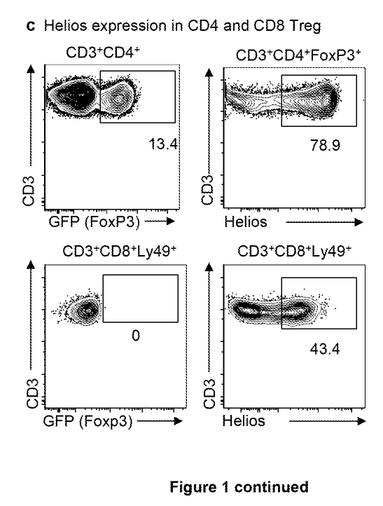

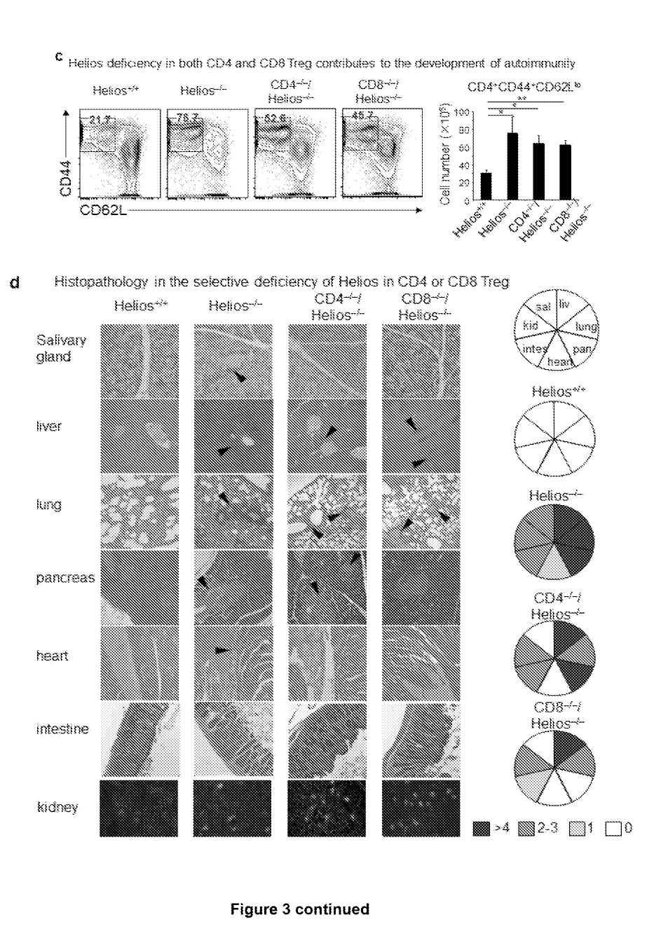

The question of whether Helios deficiency might result in a breakdown of self-tolerance was investigated. Helios deficient mice have diminished numbers of Ly49⁺CD8 cells while the numbers of FoxP3⁺CD4 Treg are similar to Helios WT mice (FIG. 7). We first analyzed unmanipulated mice for disease as they aged. Although an autoimmune disorder was not noted in 2-3 mo old mice, splenic CD4 and CD8 T cells from 5 mo old Helios-deficient mice displayed a highly activated phenotype and there was a ˜5 fold increase in splenic T_{FH} cells and GC B cells compared to WT mice (FIG. 2A). Autoimmune disease was apparent by 6-8 months of age and increased in intensity until approximately one year and was associated with extensive infiltration of immune cells into non-lymphoid tissues, including salivary glands, liver, lung, pancreas and kidney (FIG. 2B) and production of high levels of autoantibodies (FIG. 2C). Immunopathological analyses revealed glomerular nephritis in Helios^{-/-} mice characterized by mesangial thickening and prominent IgG deposition (FIG. 2D).

The development of autoimmunity by Helios^{-/-} mice was a direct consequence of defective Helios expression by lymphocytes and was not due to systemic effects of Helios deletion, since Rag2^{-/-} mice reconstituted with BM from Helios^{-/-} donors developed autoimmunity (FIGS. 2E-2G). Here, lymphopenic conditions accelerated progression of autoimmunity: Rag2^{-/-} mice reconstituted with Helio^{-/-} BM but not Helios^{+/+} BM displayed splenomegaly, increased numbers of activated CD4 and CD8 T cells and autoantibodies within 9 wks after reconstitution (FIGS. 2E-2G). These findings indicate that an intrinsic lymphocyte deficiency of Helios results in the development of autoimmunity.

Development of Autoimmunity by Helios-Deficient Mice after Viral Infection

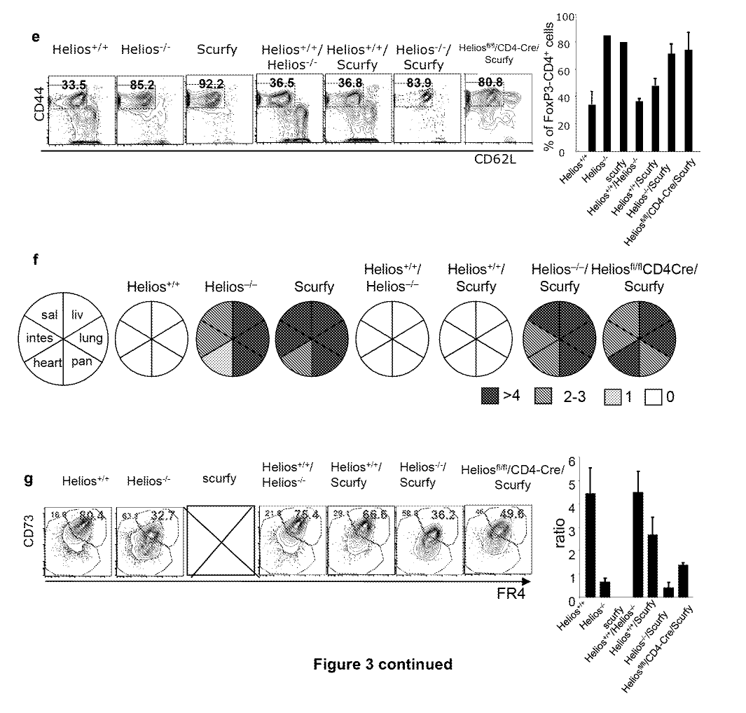

Inflammation associated with viral infection accelerates development of autoimmunity associated with defective regulatory T-cell function. Young (2 mo) and older (6 mo) Helios-deficient mice were infected with LCMV-Armstrong virus. Helios deficient mice had higher frequency of virus specific CD8 cells at day 12 post infection and both Helios WT and KO mice cleared virus efficiently (FIG. 8). Both young and older Helios deficient mice rapidly developed increased levels of T_{FH} cells and GC B cells in spleen (FIG. 2H), and high levels of IgG deposition in kidney (FIG. 2I). These autoimmune changes were similar after infection at both ages, indicating that Helios is required to maintain self-tolerance after infection even at early stages of immunological development.

### Example 4: Negative Selection is not Impaired in Helios-Deficient Mice

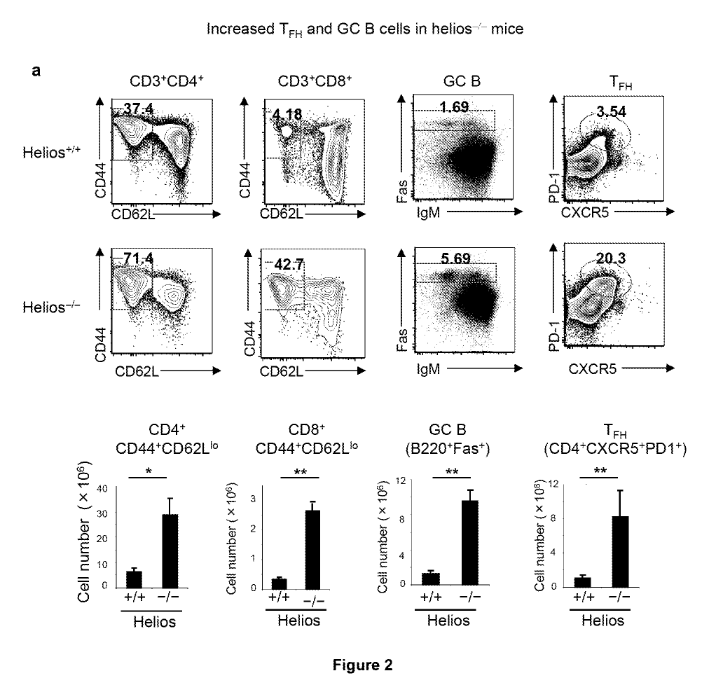

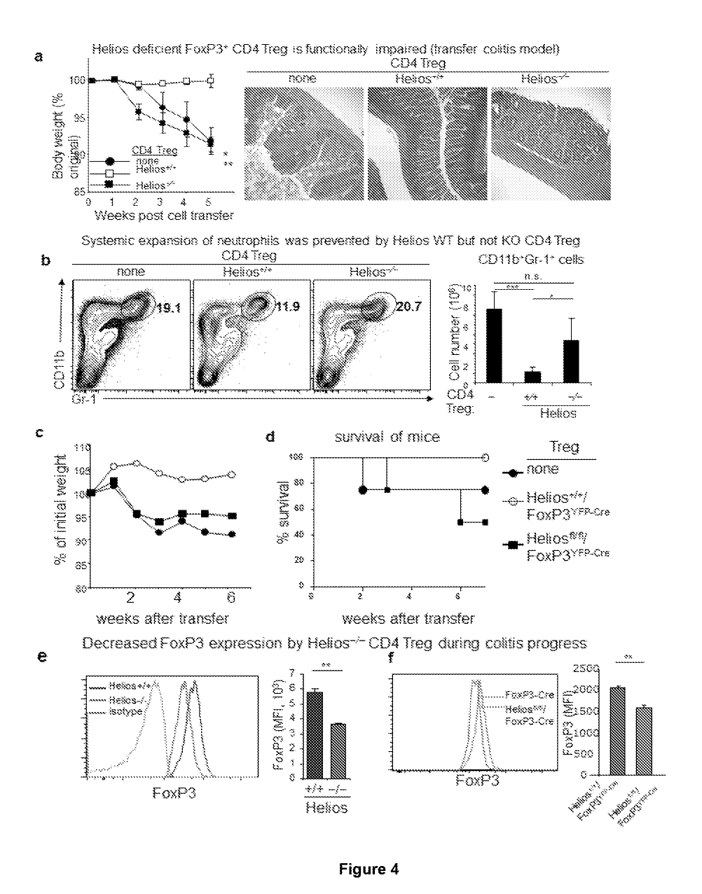

The broad spectrum of autoimmune changes observed in Helios^{-/-} mice suggested that Helios might contribute to self-tolerance through its impact on negative selection and/or the development of regulatory T cells. Activated DP thymocytes that undergo negative selection acquire the CD4^{lo}CD8^{lo} (DP^{dull}) phenotype, upregulate CD69 and express active Caspase-3 and also express Helios. To determine whether impaired negative selection of self-reactive cells may contribute to autoimmunity in Helios^{-/-} mice, the frequency of CD69⁺act-Casp3⁺ DP^{dull} cells in Helios WT and KO thymocytes was compared. This analysis showed that numbers of DPdull CD69⁺ thymocytes that expressed act-Casp3 were not affected by Helios deficiency (FIG. 3A, FIG. 9). Two fates of cells that undergo apoptosis (activated Caspase-3⁺), i.e., death by neglect (CD69⁻CD5⁻) and death by negative selection (CD69⁺CD5⁺) among CD4^{lo}CD8^{lo} thymocytes, did not show significant difference between Helios WT and KO mice (FIG. 3A ).

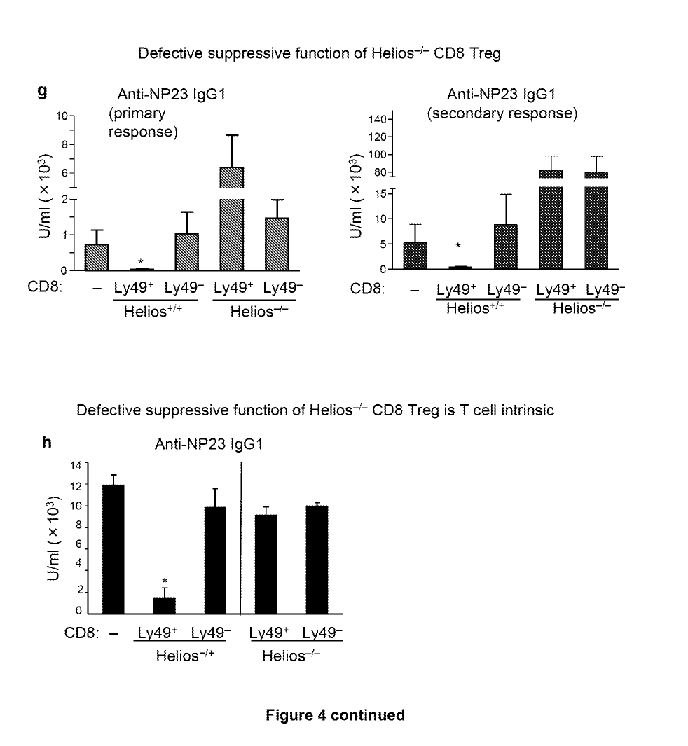

To more directly test whether Helios deficiency alters antigen induced deletion and Treg generation, OT-II and RIP-mOVA Tg system, in which promiscuous expression of a pancreatic Ag OVA in thymic medullary cells induces deletion of developing OT-II cells (Gray D H 2012), was adopted. Reconstitution of WT B6 mice with Helios WT or KO OT-II BM cells resulted in positive selection of OT-II cells independent of Helios expression in developing thymocytes (FIG. 3B, left two). In negative selection conditions of RIP-mOVA Tg hosts, percentage and number of CD4 SP cells were dramatically reduced in the thymus and spleen, reflecting negative selection of self-reactive CD4 T cells (FIG. 3B and FIG. 10). Helios deficiency in these self-reactive CD4 cells did not lead to increased percentage and numbers of CD4 SP cells, suggesting that self-reactive CD4 cells can be efficiently deleted in the Helios deficiency (FIG. 3B). These data together suggest that Helios deficiency does not impair negative selection of self-reactive CD4 T cells.

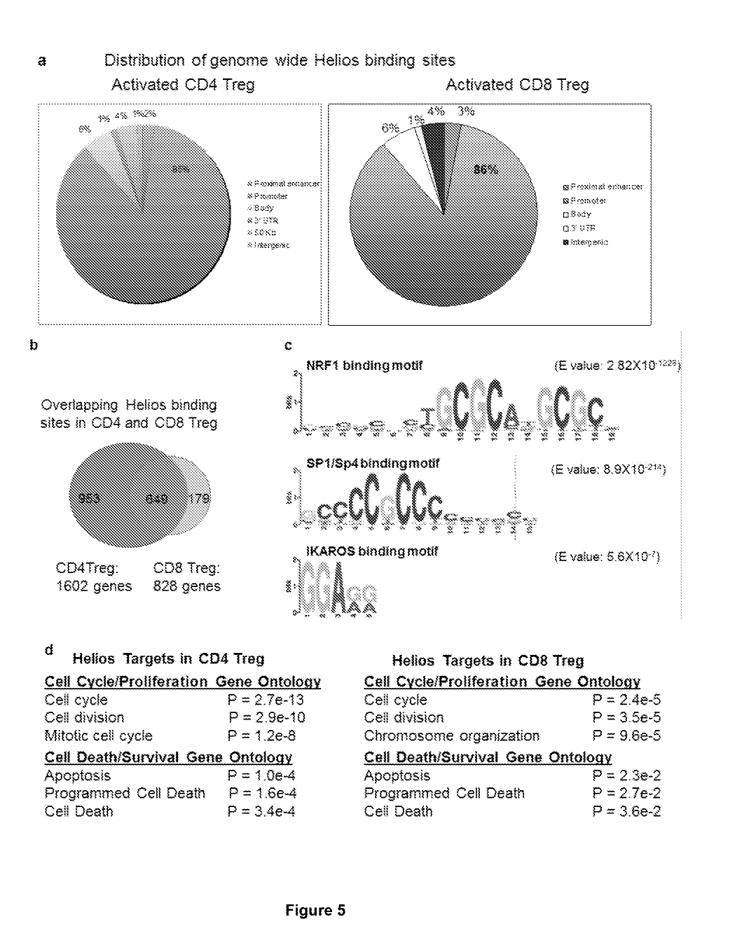

The impact of Helios deficiency on the development of self-reactive T cells into FoxP3⁺ regulatory lineage was analyzed. In the presence of self-antigen (RIP-mOVA hosts), increased fraction (˜2%) of OT-II cells developed into FoxP3+CD4 Treg lineage compared to the condition without self-antigen (WT B6 hosts). This pattern was, however, independent of Helios expression in developing thymocytes, since no reduction or increase of FoxP3⁺CD4 Treg was observed in the RIP-mOVA chimeras that were reconstituted with Helios WT or KO OT-II BM cells (FIG. 11).

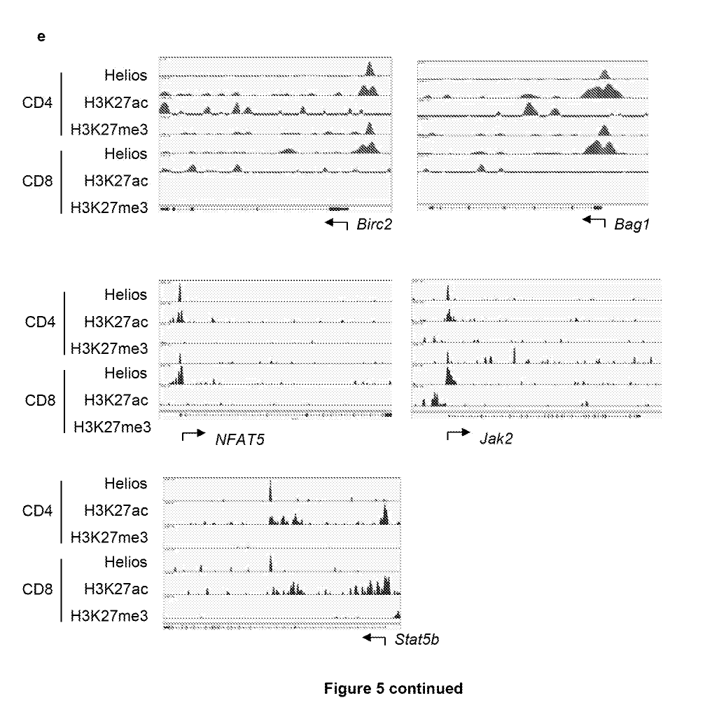

Whether Helios might contribute to negative selection of self-reactive CD8 T cells by utilizing HY TCR knock-in mice was tested. The H2-D^{b} restricted HY TCR recognizes Y-chromosome encoded self-antigen in males but not in females. The frequency of DP^{dull} HY⁺ thymocytes that expressed active caspase-3 was similar between Helios^{+/+} and Helios^{-/-} female mice. Male Helios^{-/-} mice displayed an increase in act-Casp3⁺ DP^{dull} HY⁺ cells undergoing apoptosis, but no increase in HY TCR⁺ SP thymocytes (FIG. 12).

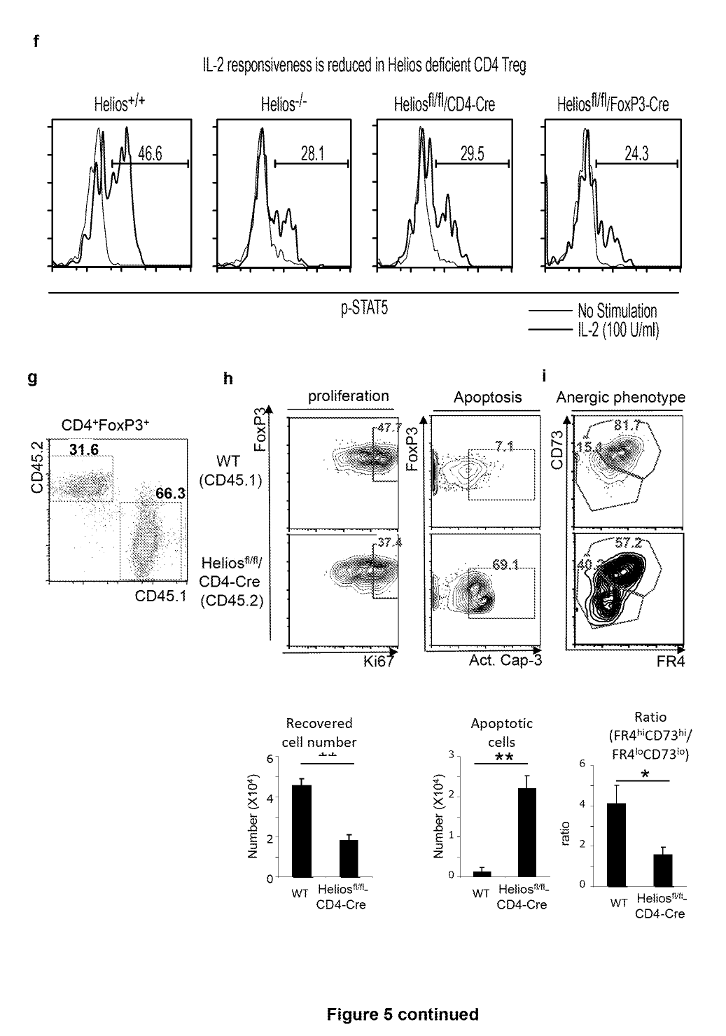

Further investigation on the potential involvement of Helios in negative selection judged by deletion of self-reactive thymocytes specific for endogenous Mtv-superantigen, an MHC class II binding peptide showed that there is no difference in the MTV-9-dependent deletion of Vβ5⁺ thymocytes in Helios WT and KO mice and there was also no accumulation of Vβ5⁺CD4 cells in the spleen of Helios deficient mice (FIG. 13), suggesting that superantigen-dependent thymic deletion is not impaired in the Helios deficiency.

These data suggest that the development of autoimmunity in Helios^{-/-} mice did not reflect a defect in negative selection.

### Example 5: Contribution of Defective CD4 vs. CD8 Treg Inhibitory Activity to Autoimmunity in Helios-Deficient Mice

Helios deficiency in CD4 and/or CD8 Treg may contribute to the breakdown of self-tolerance and development of autoimmune disease in Helios^{-/-} mice. The independent impact of Helios deficiency in CD4 or CD8 Treg using BM chimeras was investigated.

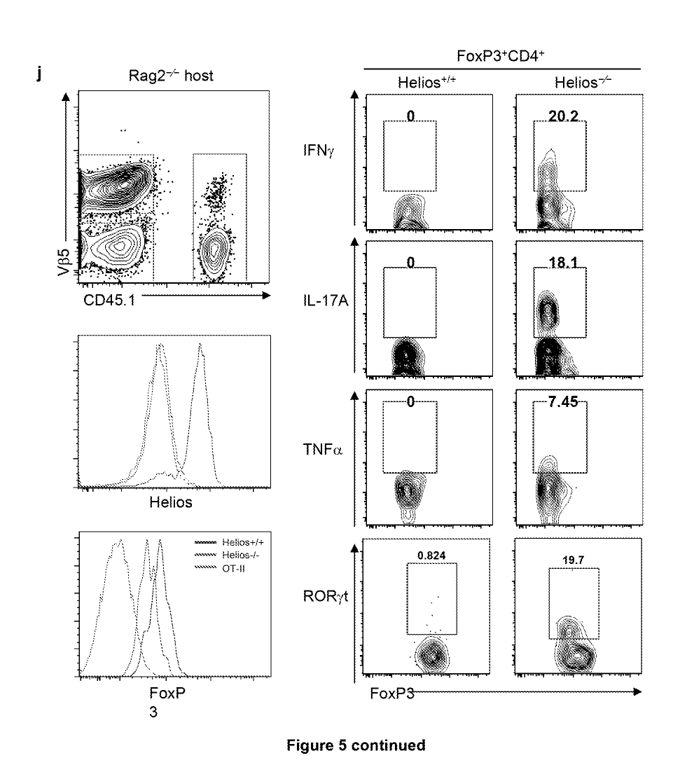

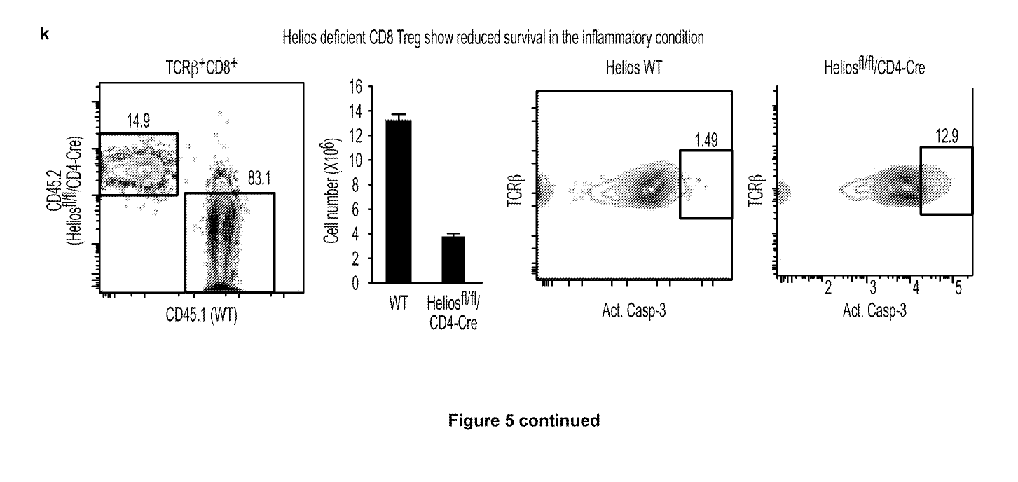

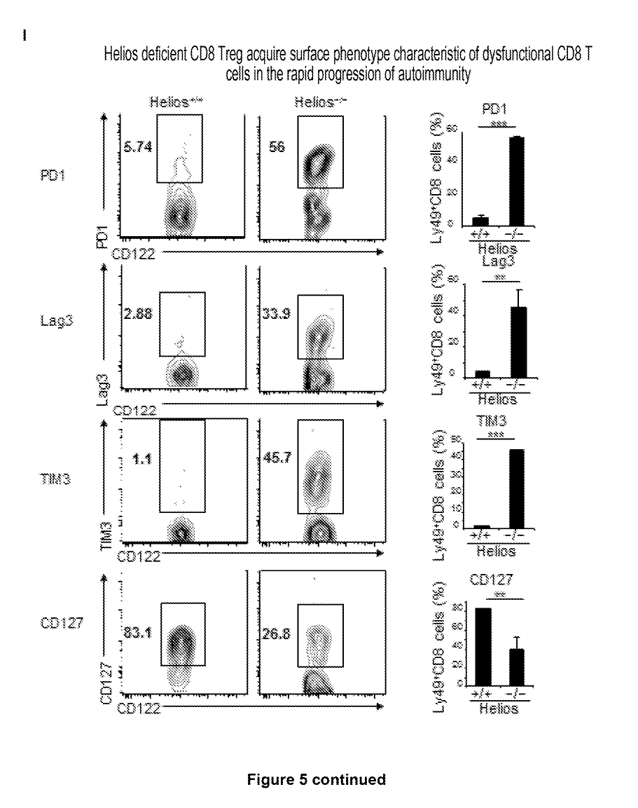

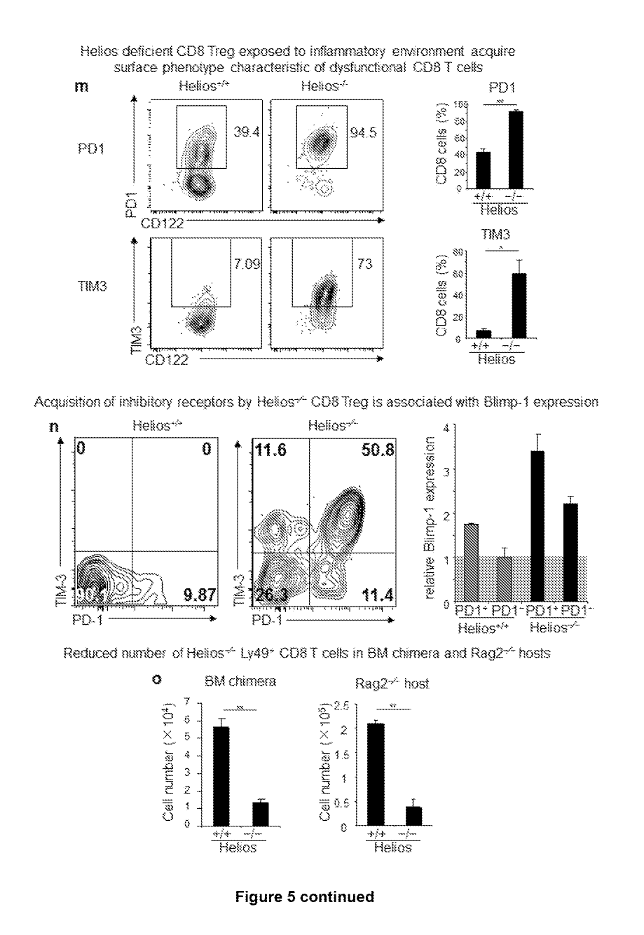

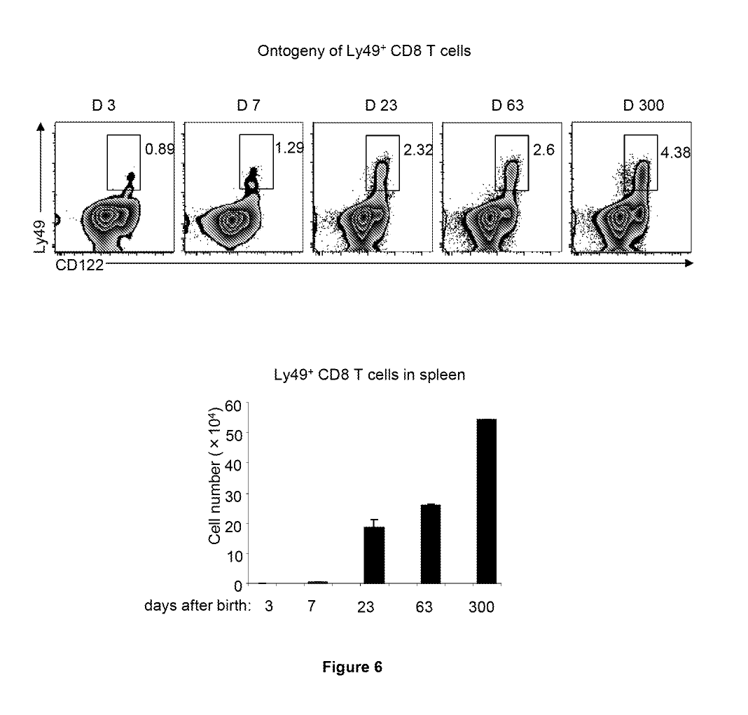

Lethally irradiated Rag2^{-/-} hosts were reconstituted with NK1.1⁺ and TCR⁺-depleted BM cells that were 1) Helios^{+/+}, 2) Helios^{-/-}, 3) 1:1 ratio of CD4^{-/-} BM+Helios^{-/-} BM, or 4) 1:1 ratio of CD8^{-/-} BM+Helios^{-/-} BM to generate mice containing lymphoid cells that were Helios sufficient, Helios deficient, or contained Helios-deficient CD4 Treg or CD8 Treg, respectively (FIG. 14). BM chimeras that were completely Helios deficient rapidly recapitulated the autoimmune phenotype of Helios^{-/-} mice, as evidenced by increased numbers of activated T cells (FIG. 3B), infiltration of immune cells into multiple organs and associated tissue damage. BM chimeras containing either Helios-deficient CD4 or CD8 Treg developed autoimmune disease with similar features (FIG. 3C). The autoimmune phenotype observed in Rag2^{-/-} hosts reconstituted with CD4^{-/-} BM+Helios^{-/-} BM or CD8^{-/-} BM+Helios^{-/-} BM cells reflected defective dominant tolerance mediated by Helios⁺CD4 or CD8 T cells respectively, since reconstitution of Rag2^{-/-} mice with Helios^{-/-} BM as well as Helios^{+/+} BM cells failed to develop autoimmune disorder (FIGS. 15 and 16).

The potential impact of Helios deficiency in FoxP3⁺CD4 Treg on development of autoimmune disease was tested by establishing BM chimeras that allow selective deletion of Helios in FoxP3⁺CD4 cells. Lethally irradiated Rag2^{-/-} mice were reconstituted with Helios^{+/+}, Helios^{-/-}, Scurfy, Helios^{+/+}/Helios^{-/-}, Helios^{+/+}/Scurfy (1:1), Helios^{-/-}/Scurfy (1:1) and Helios^{fl/fl}/CD4-Cre/Scurfy BM cells. While BM chimera reconstituted with Helios^{+/+}, Helios^{+/+}/Helios^{-/-} and Helios^{+/+}/Scurfy BM cells showed no signs of autoimmune disease, BM chimeras generated with Helios^{-/-}, Scurfy, Helios^{-/-}/Scurfy (1:1) and Helios^{fl/fl}/CD4-Cre/Scurfy BM cells rapidly developed autoimmune disease characterized by development of wasting disease, high levels of CD4 T cell activation and infiltration of immune cells into multiple peripheral organs (FIGS. 3E-3F, and FIG. 16).

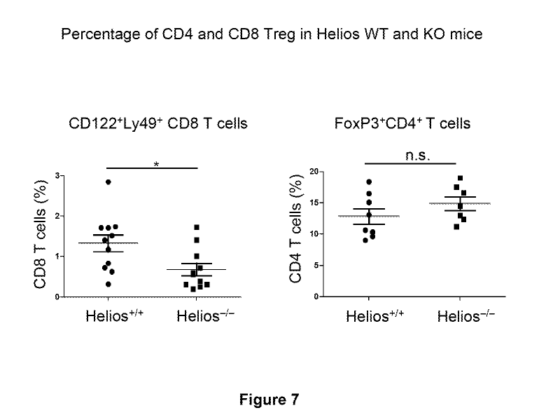

Close inspection of FoxP3⁺CD4 Treg in spleens of these BM chimeras revealed that Helios deficiency in FoxP3⁺CD4 Treg under the inflammatory conditions resulted in reduced numbers of FoxP3⁺CD4 cells and decreased expression of FR4 and CD73 that reflects non-anergic Treg phenotype compared to mice with Helios sufficiency (FIG. 3G and FIG. 16). Additional evidence that Helios dependent maintenance of CD4 Treg integrity is essential for the prevention of autoimmune disease came from analysis of Helios^{fl/fl}/FoxP3-Cre mice. BM chimeras generated with BM cells from Helios^{fl/fl}/FoxP3^{YFP}-Cre mice rapidly developed autoimmune disease evidenced by high levels of CD4 T cell activation and immune cell infiltration into multiple non-lymphoid organs. FoxP3⁺CD4 T cells in spleens of these mice with autoimmune disease displayed non-anergic phenotype characterized by reduced FR4 and CD73 expression (FIG. 19).

These results suggest that Helios-dependent regulatory activity exerted by both CD4 and CD8 Treg might be necessary for optimal maintenance of self-tolerance.

### Example 6: Helios and CD4 Treg Function

Although the contribution of Helios to the development and function of CD4 Treg is not obvious in young non-immunized mice maintained under SPF conditions, its contribution to maintenance of the CD4 Treg phenotype in a lymphopenic or inflammatory environment, or as a consequence of aging were investigated.

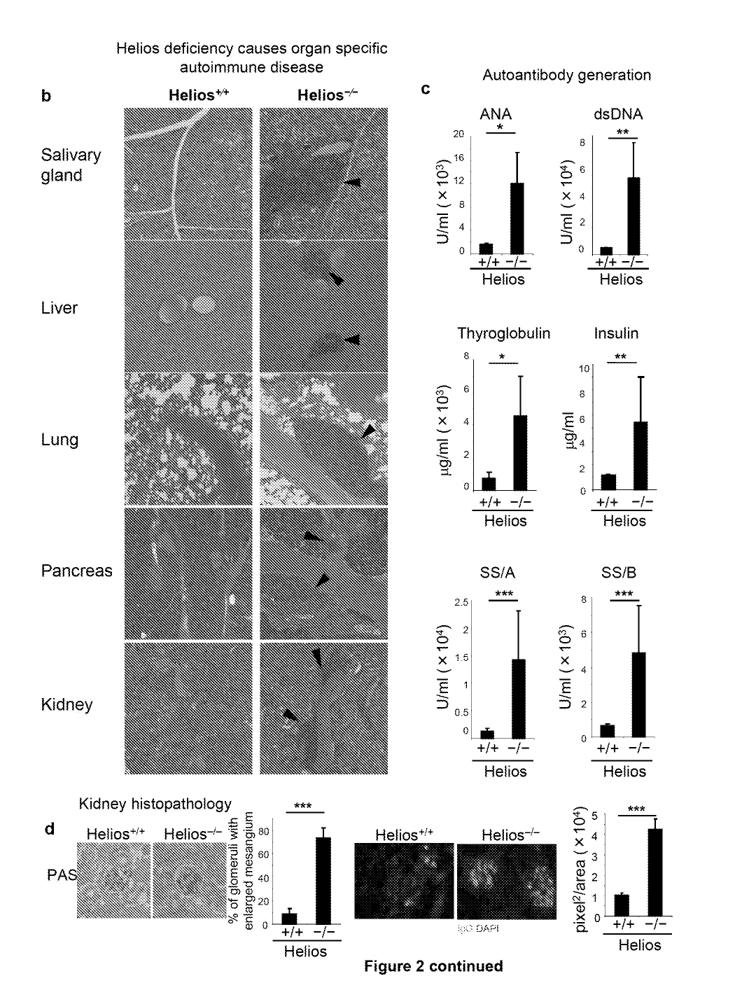

The regulatory activity of Helios^{-/-} CD4 Treg in a transfer model of colitis was investigated. Recipients of naïve CD4 effector T cells developed wasting disease and colitis within 4 wks, that was prevented by Helios^{+/+} FoxP3⁺CD4 Treg but not affected at all by co-transfer of Helios^{-/-} FoxP3⁺CD4 Treg (FIG. 4A). Defective inhibitory activity of Helios^{-/-} CD4 Treg was also signified by systemic inflammation as judged from a robust expansion of CD11b⁺Gr-1⁺ cells (FIG. 4B). Impaired suppressive activity of Helios deficient FoxP3+CD4 Treg could be also observed when FoxP3⁺CD4 cells (YFP⁺) from Helios^{fl/fl}/FoxP3^{YFP}-Cre were transferred into Rag2^{-/-} mice, which resulted in death of mice from development of wasting disease (FIG. 4C). Helios deficient CD4 Treg recovered from Rag2^{-/-} hosts displayed decreased FoxP3 expression (FIGS. 4E-4F).

Results from the comparison of gene expression between Helios⁺ and Helios^{-/-} FoxP3⁺CD4 Treg indicate Helios⁺CD4 Treg may be superior to Helios^{-/-} counterpart in their suppressive activity by expressing increased levels of KLRG1, Granzyme B, ICOS and IL-10.

Although Helios belongs to a set of critical transcriptional regulators of the FoxP3⁺ Treg genetic program, unlike other Ikaros family members (Ikaros, Eos and Aiolos), Helios does not form protein complexes with FoxP3. ChiP-seq analysis showed that Helios does not bind to FoxP3 gene locus (FIG. 19).

These observations suggest that Helios may also contribute to FoxP3⁺ Treg activity through mechanism(s) distinct from direct regulation of FoxP3 expression. Reduction of FoxP3 expression by Helios deficient CD4 Treg under inflammatory conditions may be due to indirect molecular consequence initiated from reduced IL-2 responsiveness leading to diminished activation of STAT5 that may impair CNS-2-mediated stable inheritance of FoxP3 expression and thereby promotes differentiation of CD4 Treg into inflammatory effector cells.

### Example 7: Helios and CD8 Treg Function

The question of whether Helios deficiency results in defective CD8 Treg function was investigated. Helios-deficient Ly49⁺CD8 cells contain the DX5^{hi}VLA4^{hi} subset of Ly49⁺CD8 cells found in Helios WT mice, as judged by expression of surface markers characteristic of Helios⁺Ly49⁺ cells, indicating that Helios deficiency does not abolish development of Ly49⁺CD8 T cells. To examine the ability of Helios deficient CD8 Treg to inhibit T_{FH} cells, CD25-depleted CD4 cells were co-transferred along with Ly49⁺ or Ly49⁻ CD8 cells from Helios WT or KO mice. Adoptive Rag2^{-/-} hosts were infused with Helios WT B cells and immunized with NP₁₉-KLH in CFA and challenged with NP₁₉-KLH/IFA 10 days later. Helios sufficient Ly49⁺CD8 cells efficiently suppressed anti-NP Ab responses while Ly49⁻ CD8 cells did not (FIG. 4G). Both Ly49⁺ and Ly49⁻ CD8 cells from Helios^{-/-} mice failed to transfer suppressive activity. Indeed, Ly49⁺ (and Ly49⁻) CD8 cells from Helios^{-/-} mice enhanced Ab responses compared to mice without CD8 T cells (FIG. 4G), suggesting that Helios expression by Ly49⁺CD8 cells was essential to their regulatory activity. Moreover, the contribution of Helios to CD8 Treg activity did not require expression by non-lymphoid cells. Ly49⁺ but not Ly49⁻ CD8 cells isolated from Rag2 recipients that had been reconstituted with Helios WT BM cells mediated inhibitory activity, while this inhibitory activity was absent in Ly49⁺CD8 T cells from Rag2^{-/-} recipients that had been reconstituted with Helios KO BM cells (FIG. 4H). These findings indicate that acquisition of inhibitory activity by CD8 Treg requires intrinsic expression of Helios by developing CD8 cells.

### Example 8: Helios Regulates Genes Associated with Cell Division and Survival in CD4 and CD8 Treg

To determine the genetic basis for the Helios dependent maintenance of suppressive activity by CD4 and CD8 Treg, genome-wide distribution of Helios binding sites in CD4 and CD8 Treg was measured by chromatin immunoprecipitation followed by DNA sequencing (ChiP-seq). The chromatin state of Helios-bound regions was defined by performing ChiP-seq with antibodies to two histone modifications: acetylation of histone H3 at Lys27 (H3K27ac) for active regulatory regions and trimethylation of histone H3 at Lys27 for polycomb-repressed regions.

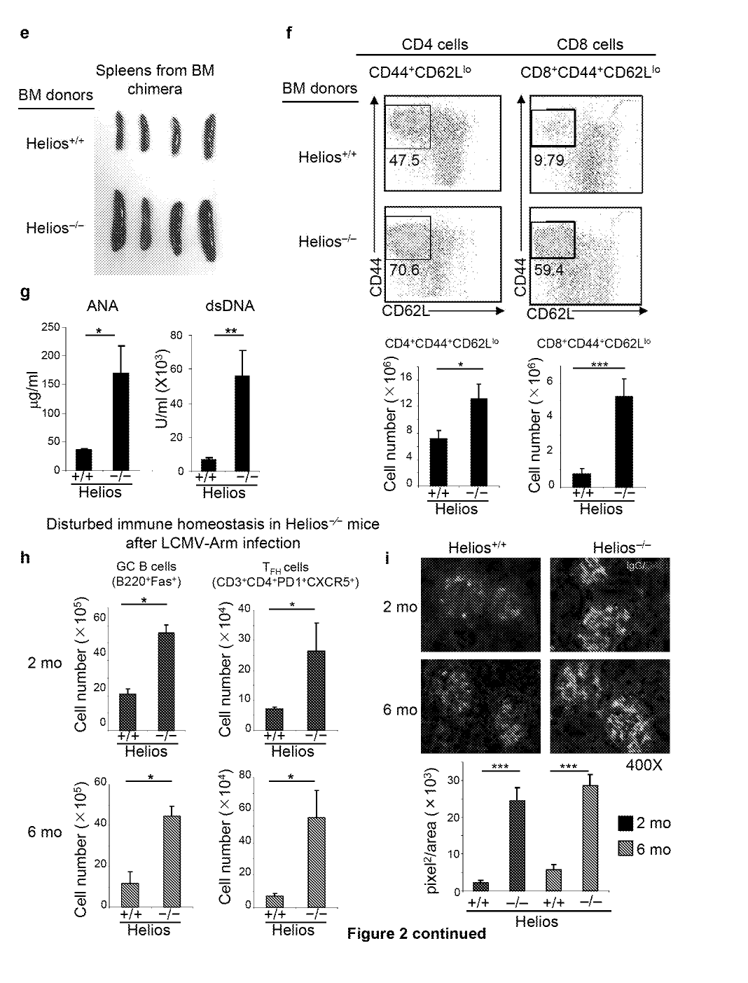

Analysis of distribution of genome wide Helios binding sites revealed that Helios mainly bound to promoter region of target genes (˜85% of target genes) in both CD4 and CD8 Treg (FIG. 5A). 1602 and 828 genes were identified as Helios target genes in CD4 and CD8 Treg respectively with 649 common target genes in both regulatory cells (FIG. 5B). Motif analysis of DNA regions bound by Helios showed considerable enrichment of NRF1, Sp1/Sp4 and IKAROS binding motifs (FIG. 5C). Inspection of these Helios target genes revealed that a significant number of genes encoded molecules with functions critical for the cell cycle progression and apoptosis/cell survival including Birc2, Bag1, NFAT5, Jak2 and Stat5b (FIGS. 5D-5E). Examples from this group of genes are provided (Table 1). Notably, Helios mainly activated genes in a similar pattern in both CD4 and CD8 Treg (FIG. 5E).

TABLE 1
Helios target genes associated with cell cycle and survival

| CD4 Treg | | | CD8 Treg | | |
|---|---|---|---|---|---|
| Aifm1 | Dpf2 | Phlda3 | Aatf | Fbxo31 | Ppp2ca |
| Atm | E2f2 | Ppm1f | Apc | Fbxo5 | Rad21 |
| Bad | Eif5a | Prkdc | Arl3 | Gramd4 | Ran |
| Bag1 | Ercc2 | Rabep1 | Bag1 | Hinfp | Rassf1 |
| Bag4 | Faim | Rad21 | Banp | Ilkap | Rbbp8 |
| Bat3 | Fastkd3 | Rnf130 | Bfar | Ints3 | Rbm7 |
| Bcl2 | Gramd4 | Rock1 | Birc2 | Lig4 | Rnf130 |
| Bfar | Il2ra | Rtn3 | Ccni | Lig4 | Rock1 |
| Birc2 | Jak2 | Shf | Cdc7 | Mapk7 | Rtn3 |
| Bnip2 | Lig4 | Sltm | Ctnnb1 | Mapk7 | Sirt7 |
| Casp2 | Luc7l3 | Srgn | Dap | Mlh3 | Smarcb1 |
| Ccar1 | Mapk7 | Stat5b | Ddx11 | Mycbp2 | Smc4 |
| Cln5 | Mef2a | Tax1bp1 | Dedd2 | Ndufa13 | Spast |
| Ctnnb1 | Nae1 | Tfdp1 | Dffb | Nisch | Stag1 |
| Dad1 | Ndufa13 | Tia1 | Dnaja3 | Nup62 | Stat5 |
| Dap | NFAT5 | Tial1 | Dpf2 | Opa1 | Stx2 |
| Dapk3 | Nisch | Tmbim6 | E2f6 | Pard6b | Tia1 |
| Dedd2 | Nup62 | Topors | Eid1 | Pds5b | Tmbim6 |
| Dffb | Opa1 | Tradd | Eif5a | Pim3 | Tradd |
| Dido1 | Pdcd5 | Wwox | Esco1 | Pkmyt1 | Tubg1 |
| Dnaja3 | Pdcd6ip | Zc3h8 | Fastkd3 | Ppm1d | Wwox |
| — | Pdcl3 | Zfp346 | | Ppp1cb | Zfp318 |

### Example 9: Mechanism of Helios Dependent FoxP3⁺CD4 Treg Stability

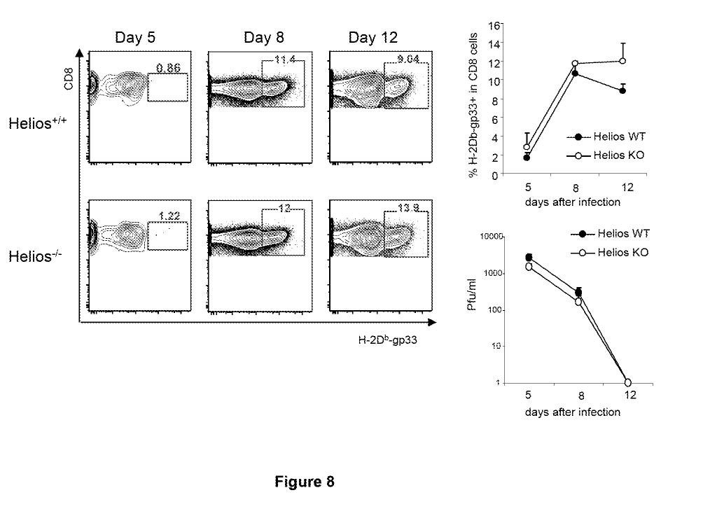

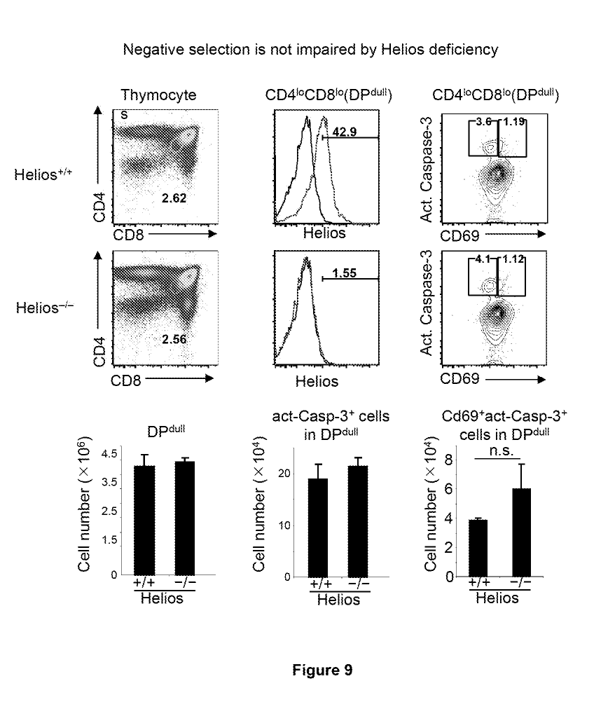

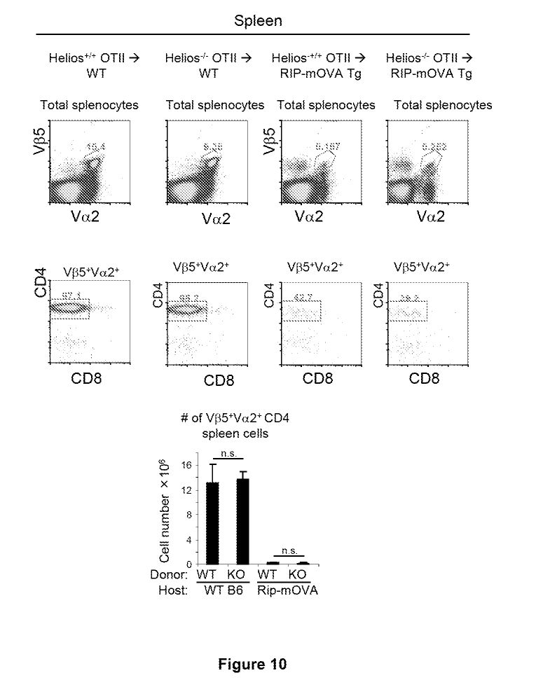

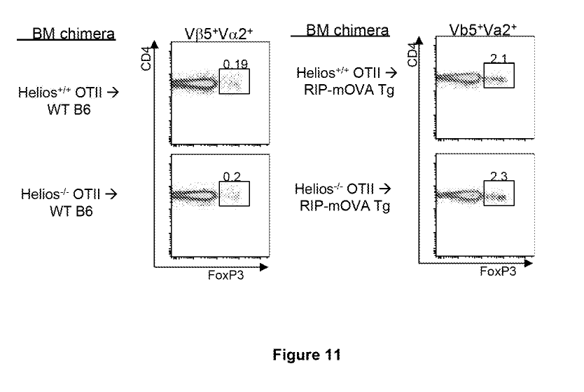

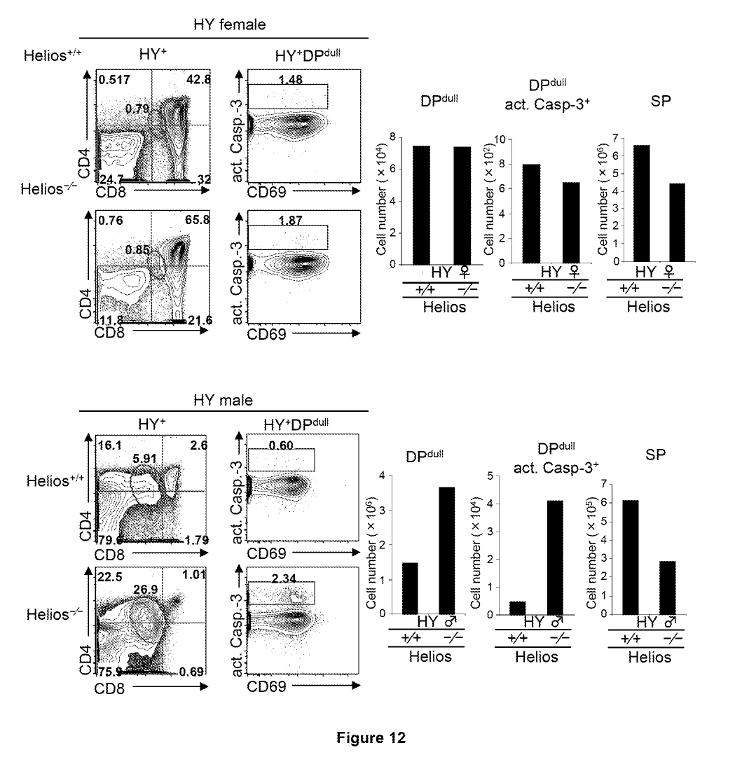

Pathway analysis with Helios target genes in CD4 Treg indicated that Helios regulates genes involved in IL-2 signaling and sustained survival (FIG. 20). Whether FoxP3⁺CD4 Treg isolated from inflammatory conditions display differential IL-2 responsiveness in the presence or absence of Helios was tested. Helios deficient FoxP3⁺CD4 Treg from BM chimeras with Helios^{-/-}, Helios^{-/-}/CD4-Cre and Helios^{fl/fl}/FoxP3-Cre BM cells with developing autoimmune diseases displayed decreased IL-2 responsiveness measured by Stat5b activation (FIG. 5F). Moreover, Helios deficient CD4 Treg displayed competitive disadvantage in the IL-2 limited lymphopenic conditions (Rag−/−rc−/− mice) noted by reduced cell recovery. A competitive disadvantage of Helios deficient CD4 Treg in lymphopenic condition can be the consequence of reduced proliferation, or survival or reduced lineage integrity. Analysis showed that Helios deficient CD4 Treg displayed increased cell death and acquisition of non-anergic surface phenotype (FR4^{lo}CD73^{lo}>FR4^{hi}CD73^{hi}) rather than reduced proliferation (FIG. 5G). Reduced expression of FR4 and CD73 by Helios deficient FoxP3⁺CD4 Treg in the inflammatory conditions reflected loss of CD4 Treg integrity: Helios deficient CD4 Treg recovered from Rag2^{-/-} mice with active colitis showed effector cytokine production that is associated with reduced expression of FR4 and CD73 by FoxP3⁺CD4 cells (FIG. 21). Acquisition of non-anergic phenotype by Helios deficient CD4 Treg was also observed from other inflammatory conditions including viral infection with LCMV-Arm and development of autoimmune disease (FIGS. 22-23).

Decreased Stat5 activation and acquisition of non-anergic surface phenotype by Helios deficient CD4 Treg raised the question of whether Helios prevents acquisition of alternative cell fates. Stat5 binds to FoxP3 locus (CNS2) and this stabilizes FoxP3 expression in preventing differentiation of CD4 Treg into effector cells. Whether expression of the Helios TF contributes to the maintenance of FoxP3⁺ Treg stability in the face of acute inflammation was tested. Rag2 hosts reconstituted with OT-II CD4 cells and immunized with OT-II peptides in CFA were given a mixture of CD45.1⁺ Helios^{+/+} and CD45.2⁺ Helios^{-/-} CD4 Treg. Helios^{-/-} FoxP3⁺CD4 Treg, but not Helios^{+/+} FoxP3⁺CD4 Treg, produced effector cytokines, including IFNγ, IL-17 and TNFα, and expressed the RORγt TF associated with expression of the T_{H}17 phenotype (30-32) (FIG. 5J). There was no detectable expression of the T-bet and GATA-3 TF and no detectable production of the IL-4 and IL-10 cytokines observed. In this analysis, the potential contribution of contaminant non-FoxP3⁺CD4 Treg was excluded by gating solely on FoxP3⁺CD4 T cells in CD45.1⁺ or CD45.2⁺CD4 cells recovered from Rag2^{-/-} hosts. Helios^{-/-} but not Helios^{+/+} Treg also displayed reduced FoxP3 expression (FIG. 5J). These data suggested that the contribution of Helios to FoxP3⁺CD4 Treg lineage stability reflects, in part, prevention of acquisition of alternative cell fates by survived Tregs in the face of inflammatory environments.

These findings, taken together, indicate that Helios makes a critical contribution to the stability of FoxP3⁺CD4 Treg by ensuring their survival and lineage integrity under conditions of lymphopenia, inflammation, autoimmunity and aging.

### Example 10: Mechanism of Helios Dependent CD8 Treg Stability

Whether defective suppressive activity by Helios-deficient CD8 Treg was associated with a phenotypic lability under inflammatory or lymphopenic conditions was investigated. Purified Ly49⁺CD8 cells from Helios WT (CD45.1) and Heliosfl/fl/CD4-Cre mice were transferred into Rag2−/−Prf−/− mice along with OT-II cells followed by immunization with OT-II peptide in CFA. Helios deficient CD8 Treg exhibited increased apoptosis under inflammatory condition resulting in reduced recovery compared to Helios WT CD8 Treg, which was also observed in Helios deficient FoxP3⁺CD4 Treg (FIG. 5K).

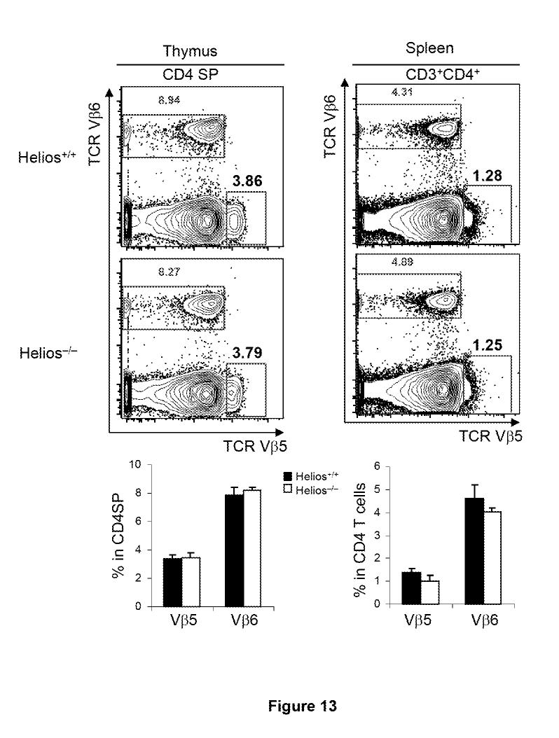

Ly49⁺CD8 Treg from BM chimeras reconstituted with Helios^{-/-} BM cells rapidly developed autoimmune disease (FIG. 3D) expressed high levels of PD-1, TIM-3 and Lag3 and low levels of CD127 compared to Ly49⁺CD8 cells from Helios WT BM chimeras (FIG. 5L). Expression of the PD-1-TIM-3 pair has been associated with diminished function and survival of CD8⁺ cytolytic cells and is related to loss of suppressive activity by Helios-deficient CD8 Treg. Helios^{-/-} CD8 Treg also acquired a similar dysfunctional phenotype after a short exposure to an inflammatory environment. Within 2 weeks after transfer of FACS-purified Helios^{+/+} and Helios^{-/-} CD8 Treg (>99% Ly49⁺ from 2 mo old mice) into Rag2^{-/-} mice along with OT-II cells and antigen, Helios^{-/-} but not Helios^{+/+}Ly49⁺CD8 cells expressed high levels of the PD-1 and TIM3 inhibitory receptors (FIG. 5M). These data show that Helios deficiency in CD8 Treg results in impaired survival and acquisition of terminally differentiated phenotype. Whereas phenotypic lability of Helios deficient CD8 Treg was associated with up regulation of exhaustion markers PD-1 and TIM-3 combined with increased apoptosis under inflammatory conditions, Helios deficient FoxP3⁺CD4 Treg rather decreased expression of activation markers PD-1 and TIM-3 (FIG. 25), which indicates that Helios dependent survival and maintenance of Treg integrity in both Tregs can result in distinct phenotype.

The Blimp-1 TF can regulate effector and memory CD8 T-cell fate through inhibition of genes associated with memory and promotion of terminal differentiation by effector CD8 cells. Whether lack of suppressive activity (FIG. 4F) and acquisition of a terminally differentiated phenotype by Helios^{-/-} CD8 Treg was associated with up regulation of Blimp-1 expression was investigated. Approximately ⅔ of Helios^{-/-} but not Helios^{+/+}CD8 T cells recovered from Rag2 hosts expressed the PD-1-TIM-3 inhibitory receptor pair. Expression of Blimp-1 was upregulated by PD-1⁺-TIM-3⁺ Helios^{-/-} CD8 cells and expression of this TF correlated with the degree of PD-1-TIM-3 expression by CD8 T cells recovered from Rag2^{-/-} hosts (FIG. 5N). Reduced numbers of Helios^{-/-} Ly49⁺CD8 cells recovered from Rag2^{-/-} hosts after transfer of Helios^{-/-} Ly49⁺CD8 cells or their BM precursors (FIG. 5O) suggested that expression of the PD-1-TIM-3 surface phenotype under inflammatory or lymphopenic conditions was associated with a diminished capacity for expansion and/or survival. Taken together, these data suggest that inhibition of Blimp-1 expression and the associated TIM-3-PD-1 surface phenotype in inflammatory or autoimmune environments depends on Helios expression by CD8 Treg.

CD8 Treg (CD44⁺CD122⁺Ly49⁺) recognize target TH cells through a Qa-1/peptide-TCR interaction and Qa-1-dependent suppression of pathogenic CD4 cells contributes to prevention of autoantibody mediated autoimmune disease. Data indicate that Helios expression is essential for regulatory activity of Ly49⁺CD8⁺ cells through maintenance of their survival and stable phenotype under inflammatory conditions. Helios deficiency in CD8 Treg results in up regulation of Blimp-1, acquisition of inhibitory receptors including PD-1, Lag3 and TIM3 and down regulation of IL-7Ra (CD127) (FIG. 5), which are associated with T cell exhaustion and loss of cytolytic function. Blimp-1 is known to regulate the terminal differentiation of diverse cell types and can promote terminal differentiation of CD8 cells at the expense of their potential to remain in the memory pool. Emergence of traits characteristic of terminal differentiation by Helios-deficient Ly49⁺CD8 cells suggests that Helios is critical for the maintenance of the central memory and suppressive phenotype of CD8 Treg. The PD-1 pathway is known to affect survival and/or proliferation of exhausted CD8 T cells, while Lag3 expression may negatively regulate T cell expansion and inhibit cell cycle progression. TIM-3 is expressed by terminally-differentiated T_{H}1 and T_{C}1 cells and its engagement can trigger cell death, while loss of IL-7Ra by Helios-deficient CD8 Treg can lead to decreased survival. Therefore, acquisition of PD-1, Lag3 and TIM3 receptors and decreased CD127 expression by Helios^{-/-} CD8 Treg activates exhaustive differentiation rather than a memory program resulting in diminished long-term survival under inflammatory conditions, which underlies the defective suppressive activity of Helios^{-/-} CD8 Treg.

Acquisition of the PD-1-TIM3 phenotype does not reflect dysregulated expansion by Ly49⁺ Helios CD8 cells secondary to defective inhibitory activity of Helios^{-/-} CD8 Treg, since these cells did not expand in the absence of functional Ly49⁺CD8 Treg (FIG. 5N). The Ly49⁺CD8 cells that express an exhausted phenotype also express DX5 and VLA4, which are co-expressed by Helios⁺Ly49⁺ cells in Helios WT mice (data not shown). Moreover, we have recently shown that defective CD8 Treg activity results in a marked decrease in the numbers of conventional anti-viral CD8 T cells that display a PD1⁺-TIM3⁺‘exhausted’ phenotype after viral (LCMV) infection and inflammation. These observations indicate that one consequence of Helios deficiency is up regulation of Blimp-1, expression of inhibitory receptors by Ly49⁺CD8 Treg characteristic of an exhausted CD8 phenotype, resulting in diminished survival and impaired suppressive activity.

Taken together, results demonstrate that Helios is essential to maintenance of immunological self-tolerance through its contribution to the regulatory activity of both CD4 and CD8 Treg. Helios is critical to the stabilization of both regulatory T cell pool in the face of excessive inflammation by ensuring survival of these T cell lineages. Helios dependent maintenance of Treg integrity involves preservation of anergic phenotype in the inflammatory condition and repression of effector cytokine expression by FoxP3⁺CD4 Treg and inhibition of terminal differentiation and maintenance of cytolytic activity by CD8 Treg respectively.

### Example 11: Identification of Targets in CD4 and CD8 Treg for Cancer Immunotherapy

Data presented in the disclosure indicate that the Helios transcription factor (TF) is expressed by both FoxP3⁺CD4 Treg and Qa-1-restricted CD8 Treg (CD122⁺Ly49⁺), but not by conventional T-cells. Expression of Helios is essential for maintenance of a stable suppressive and anergic phenotype by both regulatory lineages in the face of immune or inflammatory responses to tumors. This example describes a strategy to identify molecular targets expressed by Treg that induce Treg instability, reduction of Helios and Treg conversion into effector CD4 cells.

Isolated pure populations of homogeneous Treg cells (FoxP3^{RFP} IFNγ^{YFP} Helios^{hCD2}) are for differentiation into CD4 Teff cells upon antibody engagement of Treg target receptors that include but are not limited to anti-GITR, anti-OX-40, anti-4-1BB, anti-CD47 and anti-Nrp-1. Conversion of CD4 Treg into CD4 Teff cells by this method results in several beneficial effects obtained through (a) reduction or elimination of CD4 Treg activity and (b) conversion of Treg into high affinity effector anti-tumor cells equipped with destructive anti-tumor immune activity, and (c) conversion is confined to intratumoral Treg and spares the systemic Treg population.

In view of the negative impact of both CD4 and CD8 Treg on anti-tumor immunity, antibody-mediated targeting of the signaling pathway that reduces Helios expression by intratumoral Treg represents a novel and potentially robust approach to immunotherapy.

### In Vitro Screen of Abs for Induction of Treg→Teff Transition

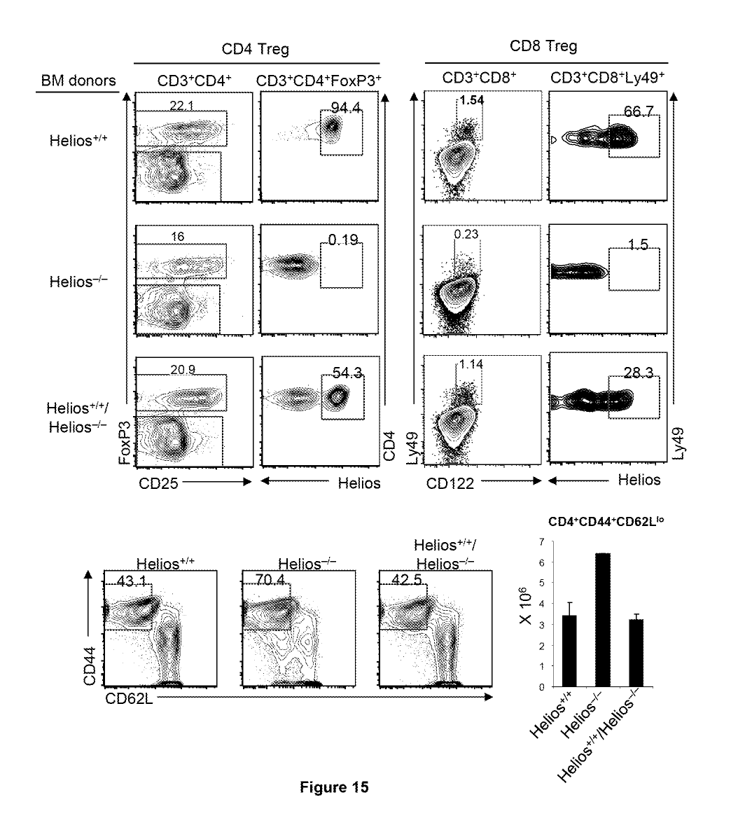

The screening method described here tests whether engagement of candidate antibodies leads to phenotypic instability of CD4 Treg in vitro. CD4 Treg isolated from B6 mice are stimulated with anti CD3 and CD28 with IL-4, IL-6 or IL-12/18. Antibodies to target molecules including but not limited to 4-1BB, Nrp1, Ox-40, CD73 and GITR are tested. The CD4 Treg phenotype is assessed with special emphasis on expression levels of FoxP3 and Helios as well as effector cytokine production. Analysis is focused on the conversion of CD4 Treg to Th1 (IFNγ producer), Th17 (IL-17 producer) and CD4-CTL (GzmB). Data show that Helios deficient CD4 Treg express effector cytokines and also a transcription factor (RORgt) that is specific for effector Th cells (e.g., IL-17 or IFNγ) (FIG. 27).

This in vitro study is performed using both mouse and human CD4 Treg. Mouse CD4 Treg are obtained from spleens of B6 mice. Controls include Treg that express reduced levels of Helios secondary to conventional Cre-mediated gene deletion. Human CD4 Treg are isolated from peripheral blood of healthy donors by labeling cells with CD25 and CD45RA and/or CCR4 to exclude contamination of FoxP3⁺ conventional CD4 cells.

### In Vitro Screening Strategy for Treg to Teff Conversion

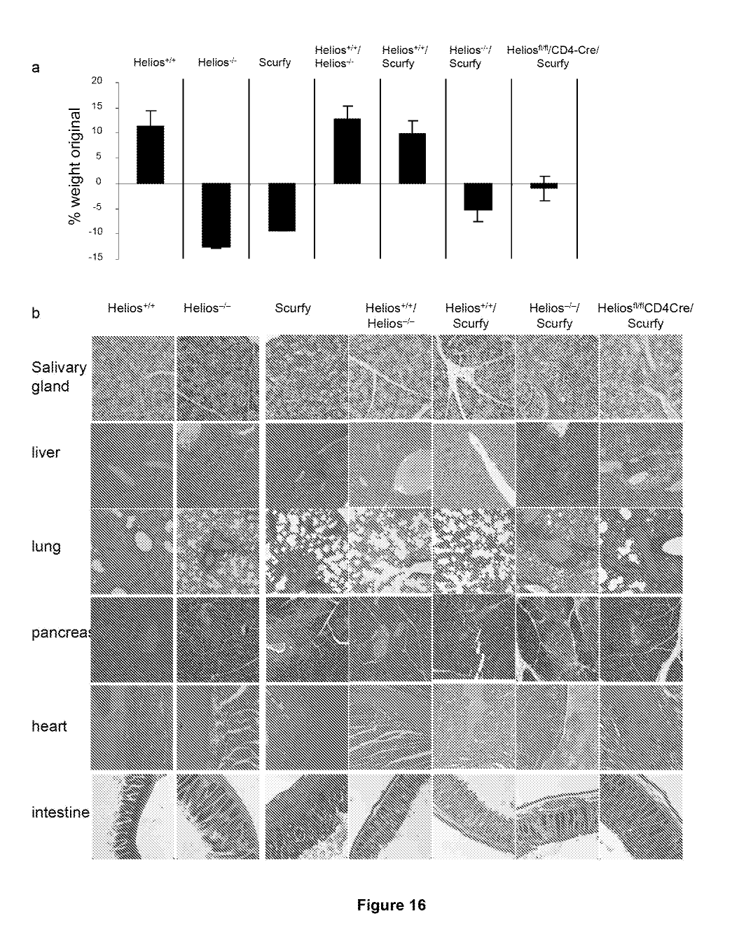

This screening system is based on the combination of three reporter mice that allows stable Treg and converted Treg to be differentiated using simple FACS analysis (FIG. 28). CD4 Treg are isolated from mice according to RFP and hCD4 expression. Cells are plated in a 96 well plate that is coated with anti-CD3 and anti-CD28. 20 ng/ml recombinant mouse IL-2 is added to each well. Candidate antibodies specific for surface molecules highly expressed by CD4 Treg are added at time 0. After 4-5 days, the plate is screened for RFP, GFP and hCD2 expression. Reduction of RFP expression is inversely correlated with increase of YFP. Correlation between reduction of RFP/increase of YFP and decrease of hCD2 is variable, since some antibodies can induce CD4 Treg to Teff conversion in a Helios independent manner.

Increase of RFP and hCD4 expression can be analyzed in parallel, to identify candidate antibodies that enhance CD4 Treg phenotype.

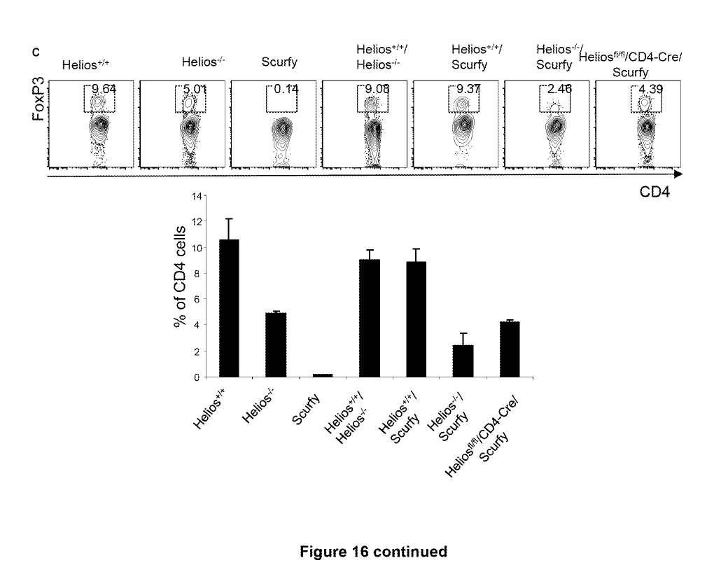

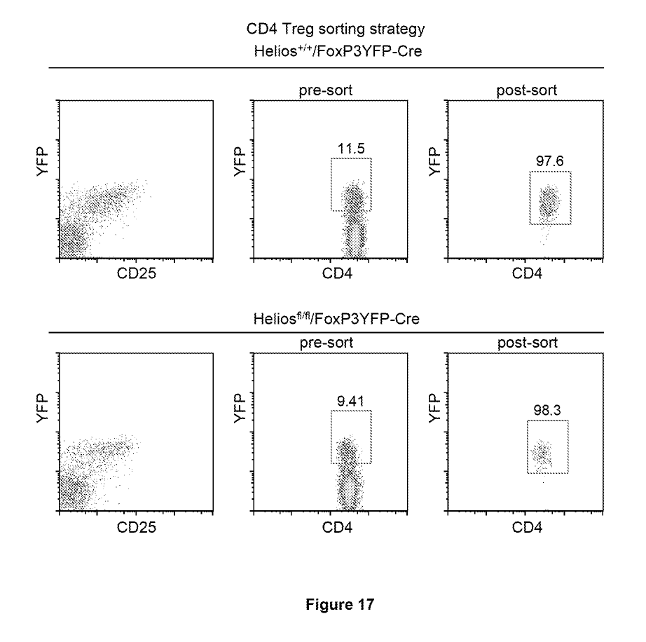

FIG. 30 provides one example of a candidate antibody that induces Treg to Teff conversion. CD73 is an ectoenzyme that hydrolyzes ADP to adenosine, is expressed by many tumor cells and also by CD4 Treg at very high levels. Analysis shows that Helios deficient CD4 Treg express reduced levels of CD73 in the inflammatory condition (FIG. 29). In addition, engagement of CD73 by antibodies in vitro in the presence of anti-CD3 and CD28 leads to down regulation of Helios and reduced Treg survival (FIG. 30). These results indicate that CD73 blocking antibody can be used to reduce CD4 Treg stability.

Agents that induce differentiation of CD8⁺ Treg will also be screened. A regulatory subset of CD8 T cells that can be identified using a triad of surface markers—CD44, CD122 and Ly49—that reliably separate the 3-5% of CD8⁺ T cells that mediate Qa-1-restricted inhibition of T helper cells have been defined. In tumor settings, inhibitory activity of CD8 Treg results in reduced antitumor immunity, which can be reversed by blocking or depletion of CD8 Treg.

Studies with B16 melanoma have shown that disruption of CD8 Treg activity results in expansion of T follicular helper (T_{FH}) cells and enhanced antitumor immunity. This finding is consistent with the contribution of Qa-1 restricted CD8 Treg in the inhibition of autoantibody mediated immune responses by targeting T_{FH} cells in the setting of autoimmune disease.

A recent analysis of the immune cell types that infiltrate human colorectal cancers during early and late stage tumor growth indicates that T_{FH} cells and B cells are the central players in long-term protection against tumor growth and strongly correlate with patient survival. Data also indicates that in the absence of CD8 Treg activity, generation of a broad range of antibodies, including TAA, allows robust anti-tumor immune responses. In this context, depletion of CD8 Treg during tumor progression may represent a promising approach for immunotherapy by inducing T_{FH} expansion and thereby boosting broad-range Ab generation.

Killer cell immunoglobulin like receptors (KIR) represent the human homologue of murine Ly49. Analysis has shown that KIR⁺CD8 T cells exhibit suppressive activity on CXCR5⁺ (T_{FH} phenotype) CD4 T cells. Studies with murine viral infection and tumor models suggest that depletion of CD8 Treg using anti-Ly49 Ab can enhance anti-viral and anti-tumor immune responses. Blocking anti-KIR Abs are available but are used mainly to enhance NK activity by blocking inhibitory signaling. No depleting Abs that specifically target CD8 Treg have been developed. A recent study has shown that the KIR repertoire in CD8 T cells in each donor is restricted toward expression of one or two dominant KIR subtypes.

NK cells also express KIR inhibitory receptors and definition of a KIR subtype that is dominantly expressed by CD8 cells is critical for targeting CD8 Treg using anti-KIR antibody. Three KIR subtypes are dominantly expressed by CD8 cells—KIR2DL1, KIR2DL3, KIR3DL1- and development of depleting Abs against these KIR subtypes may yield immunotherapeutics that are particularly useful for cancer types in which generation of a broad range of Abs for tumor associated antigens is relevant to inhibit tumor progression.

The effect of CD8 Treg depletion on tumor progression is tested using the TCL-1 lymphoma model. Treg-depleted CD4 cells with or without CD8 Treg are transferred into TCRa^{-/-} mice that have been inoculated with TCL-1 lymphoma. Lymphoma growth is monitored and the phenotype of CD4 T cells is analyzed. Since lymphoma in addition to activated CD4 cells also express Qa-1, the impact of CD8 Treg mediated suppression on lymphoma cells is tested by comparing mice transferred with Qa-1 WT or Qa-1-deficient CD4 cells.

WT B6 mice are inoculated with TCL-1 lymphoma. Ly49⁺CD8 cells are depleted from these mice by injecting antibodies one day before and every three days after tumor injection. Lymphoma growth and CD4 and CD8 T cell phenotype are monitored. Preliminary data indicate that CD8 Treg display restricted expression of KIR subtypes.

An in vitro suppression assay is performed using defined KIR subtype⁺ CD8 cells as effector cells and CXCR5⁺ memory CD4 cells as target cells. CXCR5⁺CD4 cells isolated from PBMC serve as optimal target cells for CD8 Treg, since these cells express high levels of HLA-E.

### Example 12: Instability of Helios-Deficient Tregs is Associated with Conversion to a T-Effector Phenotype and Enhanced Antitumor Immunity

Expression of the transcription factor Helios by Tregs ensures stable expression of a suppressive and anergic phenotype in the face of intense inflammatory responses, whereas Helios-deficient Tregs display diminished lineage stability, reduced FoxP3 expression, and production of proinflammatory cytokines. The data in this example show that selective Helios deficiency within CD4 Tregs leads to enhanced antitumor immunity through induction of an unstable phenotype and conversion of intratumoral Tregs into T effector cells within the tumor microenvironment. Induction of an unstable Treg phenotype is associated with enhanced production of proinflammatory cytokines by tumor-infiltrating but not systemic Tregs and significantly delayed tumor growth. Antibody-dependent engagement of Treg surface receptors that result in Helios down-regulation also promotes conversion of intratumoral but not systemic Tregs into T effector cells and leads to enhanced antitumor immunity. The following findings suggest that selective instability and conversion of intratumoral CD4 Tregs through genetic or antibody-based targeting of Helios may represent an effective approach to immunotherapy.

The transcription factor (TF) Helios is expressed by two regulatory T-cell lineages-FoxP3⁺CD4⁺ and Ly49⁺CD8⁺ Tregs—which are important for maintenance of self-tolerance (6, 7). The contribution of Helios to the maintenance of Treg size and functional stability in the face of diverse immunological perturbations is relevant to the strategies that underpin current tumor immunotherapy. Current approaches rely mainly on depletion or blockade of CD4 Tregs to shift the systemic balance toward Teff cells. However, alterations in this balance may provoke severe autoimmune disorders. The following data support approaches that selectively convert intratumoral Tregs into Teff cells without affecting the systemic Treg population.

Experimental data in this example show that selective Helios deficiency within CD4 Tregs leads to enhanced antitumor immunity through induction of an unstable phenotype by intratumoral but not systemic Tregs and conversion of these Tregs into Teff cells within the TME. Induction of an unstable Treg phenotype is associated with enhanced activity of tumor-infiltrating CD4+ and CD8+ Teff lymphocytes and significantly delayed tumor growth. Moreover, antibody-dependent engagement of Treg surface receptors that downregulate Helios expression also promote effector cell conversion of intratumoral but not systemic Tregs and enhanced antitumor immunity. These findings support a cancer immunotherapy that involves selective intratumoral inactivation and conversion of CD4 Tregs through targeting Helios.

### Intratumoral CD4 Tregs Express an Enhanced Suppressive Phenotype.

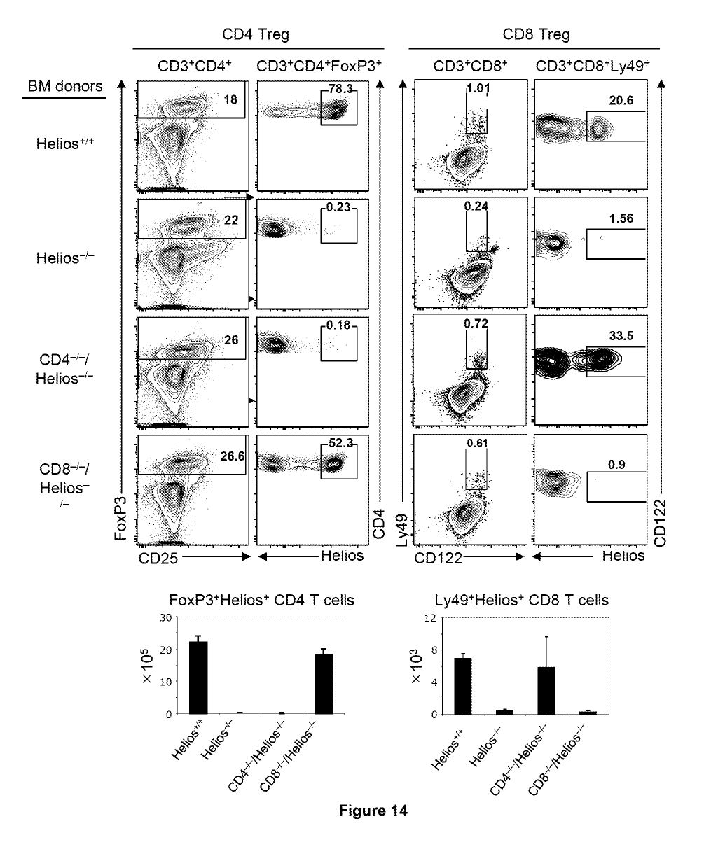

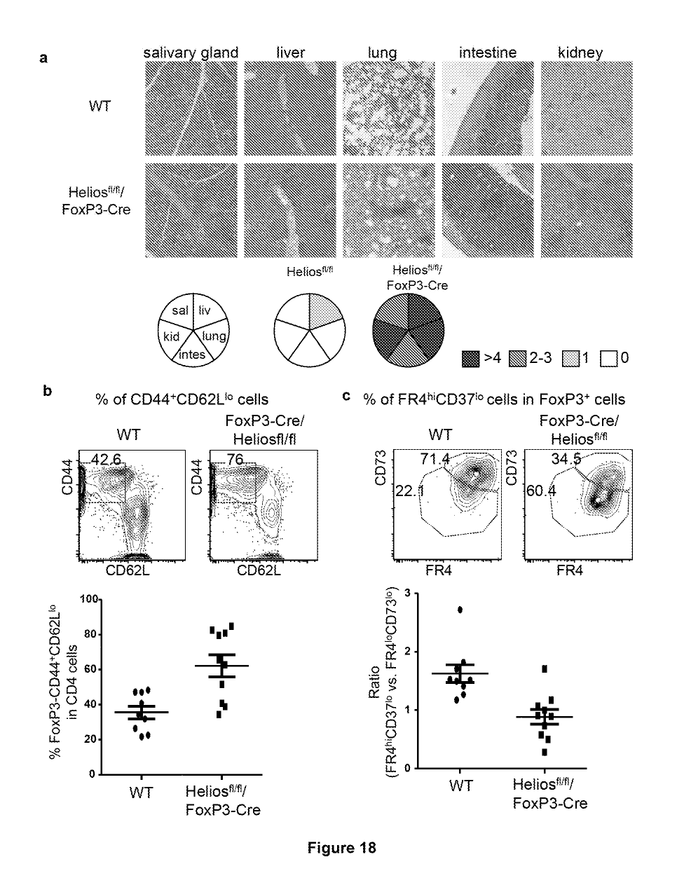

Whether the contribution of Helios to stabilization of the Treg phenotype in the face of chronic inflammatory conditions include Treg stability within progressively growing tumors was tested. A comparison of the phenotype of intratumoral Tregs with Tregs in peripheral lymphoid tissues of B16 tumor-bearing mice indicated that intratumoral CD4 Tregs expressed significantly higher levels of Helios compared with splenic or lymph node (LN) Tregs (FIG. 31A). Increased Helios expression by intratumoral Tregs may reflect preferential migration of this Treg subpopulation into tumors and/or preferential expansion and survival within the TME. In either case, increased expression of Helios by intratumoral CD4 Tregs may signal enhanced suppressive activity, as judged by expression of a gene profile associated with effector CD4 Tregs. A comparison of gene expression by Helios⁺ and Helios FoxP3⁺CD4 Tregs after separation by surrogate markers (GITR^{hi}ICOS^{hi} vs. GITR^{lo}ICOS^{lo}) revealed that Helios⁺ FoxP3⁺CD4 Tregs showed increased expression of KLRG1, GZMB, IL-10, and ICOS, i.e., molecules that are associated with robust suppressive activity (FIGS. 26A and 26B). These observations together with findings that Helios can stabilize the suppressive CD4 Treg phenotype under inflammatory but not steady-state conditions, suggest that disruption of Helios expression by intratumoral CD4 Tregs might enhance antitumor immunity.

### Selective Deletion of Helios in FoxP3+ CD4 Tregs Enhances Antitumor Immunity.

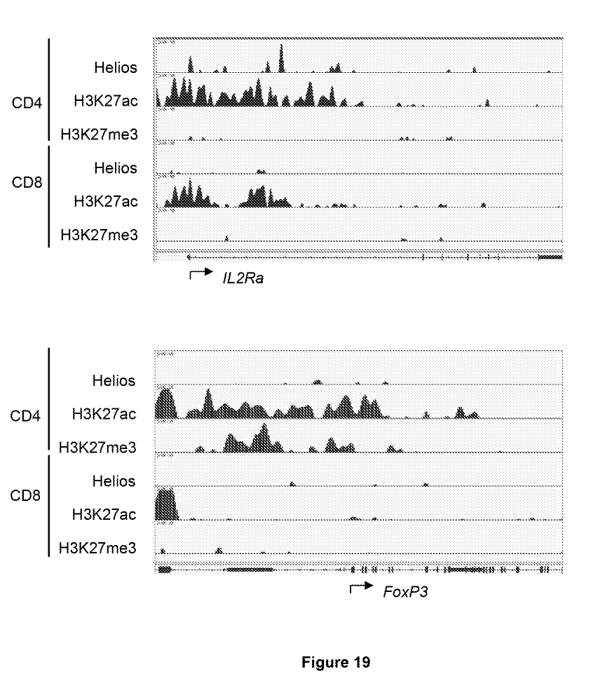

Previous studies have demonstrated that intratumoral FoxP3⁺CD4 Tregs express surface molecules, including TIM3 and TIGIT, that are associated with robust immunosuppressive activity as well as dysfunctional tumor-infiltrating lymphocytes (TILs) (3, 9). However, the impact of the Treg-specific Helios TF has not been investigated. Although Helios deficiency promotes an unstable FoxP3⁺CD4 Treg phenotype under inflammatory conditions (6), whether defective Helios expression by intratumoral Tregs impairs Treg function within this local inflammatory environment is unclear. Therefore, tumor growth in Helios-deficient (Helios^{fl/fl}.FoxP3-Cre; Helios KO) mice after s.c. inoculation of transplantable melanoma (B16/F10) or colon adenocarcinoma (MC38), was analyzed. Helios KO mice showed substantial delay of tumor growth compared with Helios WT mice, resulting in prolonged survival (FIGS. 32A and 32B), indicating that Helios expression by FoxP3⁺CD4 Tregs may normally contribute to Treg-mediated repression of antitumor immunity. Indeed, delayed tumor growth was associated with increased IFNγ production by CD8⁺ TILs in Helios KO mice compared with TILs from Helios WT mice (FIG. 32C).

Vaccination with GM-CSF-secreting irradiated tumor cells (GVAX) represents a prototypic paracrine cytokine adjuvant that induces differentiation of dendritic cells (DCs) leading to tumor antigen uptake, trafficking to tumor-draining lymph nodes, and enhanced inflammation. Because the contribution of the Helios TF to stable FoxP3+ Treg suppressive activity is critically important in inflammatory settings (6), Helios WT and Helios KO mice after s.c. inoculation with B16-Ova melanoma cells and treatment with GVAX at days 3, 7, and 9 was examined. This regimen resulted in a substantial decrease in tumor growth in Helios KO mice compared with WT mice, indicating that Helios-deficient CD4 Tregs display reduced immunosuppressive activity (FIG. 32D). Indeed, intratumoral CD4 and CD8 Teff cells in Helios KO mice expressed high levels of TNFα compared with modest levels by TILs from Helios WT mice (FIG. 32E).

Enhanced Antitumor Immunity by Helios KO Mice is Associated with an Unstable Treg Phenotype.

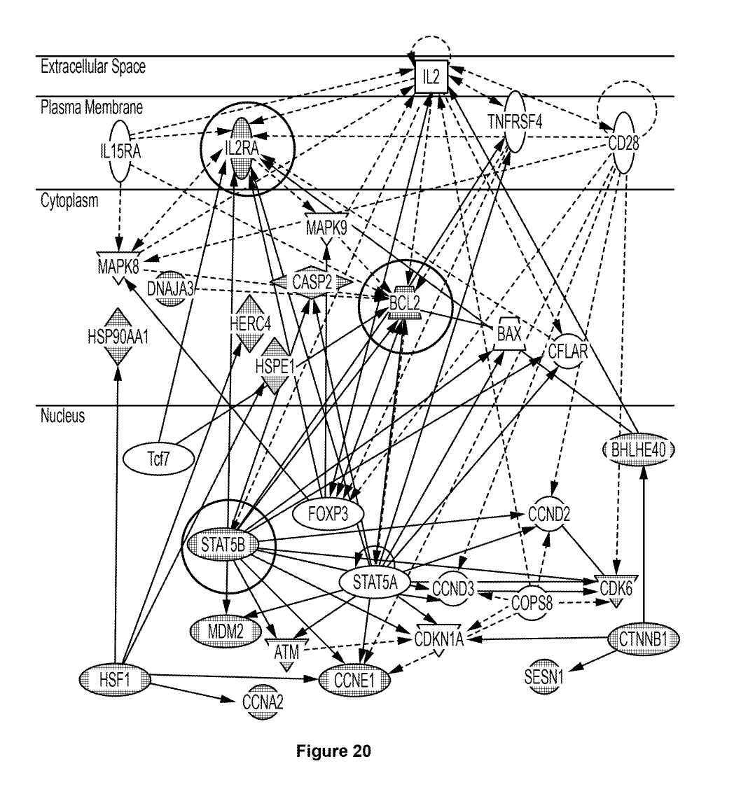

Helios-deficient CD4 Tregs develop an unstable phenotype during inflammatory responses characterized by reduced FoxP3 expression and increased effector cytokine expression secondary to diminished activation of the STAT5 pathway (6). Growing tumors attract a wide variety of cytokine/chemokine-producing leukocytes that shape the inflammatory microenvironment during tumor progression (10) and promote increased proportions of FoxP3⁺CD4 cells compared with their frequency in lymphoid tissues. Thus, ˜40% of CD4⁺ cells within B16 melanoma are FoxP3⁺, whereas ˜10% of splenic CD4 cells in tumor-bearing mice are FoxP3⁺ (FIG. 33A). However, the frequency of FoxP3⁺CD4 Tregs within CD4⁺ TILs in Helios KO mice is not increased (˜10-12%) compared with spleen (FIG. 33A). Moreover, FoxP3⁺CD4 Tregs isolated from tumors grown in Helios KO displayed a nonanergic phenotype, as judged from decreased ratio between FR4hiCD73hi (anergic) and FR4^{lo}CD73^{lo} (nonanergic) FoxP3⁺ cells (FIG. 33B). This difference was confined to intratumoral CD4 Tregs; splenic FoxP3⁺CD4 Tregs showed a similar anergic phenotype in tumor-bearing Helios WT and KO mice (FIG. 33B). Consistent with their diminished anergic surface phenotype, Helios-deficient intratumoral CD4 Tregs expressed relatively high levels of the IFNγ effector cytokine, in contrast to WT intratumoral CD4 Tregs, which displayed a stable phenotype and minimal IFNγ production (FIG. 33C). Conversion of Helios-deficient FoxP3⁺CD4 Tregs to Teff cells was also noted in B16-bearing mice that had been treated with GVAX. Helios-deficient intratumoral Tregs expressed high levels of TNFα, whereas Helios WT intratumoral CD4 Tregs produced marginal levels (FIG. 33D).

### The Impact of Helios Deficiency in CD4 Tregs on Antitumor Immunity is Cell Intrinsic.

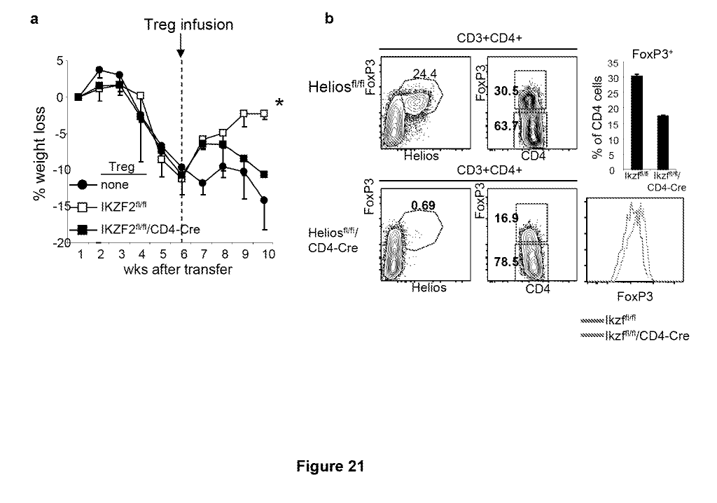

To determine whether enhanced antitumor immunity displayed by Helios KO mice is Treg intrinsic, purified Helios WT or Helios KO CD4 Tregs (CD45.2) along with CD4 and CD8 Teff cells (CD45.1) were transferred into Rag2^{-/-} hosts and monitored for tumor growth. Rapid tumor growth was observed in hosts that had received WT CD4 Tregs, whereas tumor development was delayed in hosts that had received CD4 Tregs from Helios KO mice (FIG. 34A). Analysis of CD4 Tregs recovered from adoptive hosts also revealed that Helios-deficient CD4 Tregs displayed reduced FoxP3 expression compared with Helios WT CD4 Tregs (FIG. 34B). FoxP3 down-regulation by Helios-deficient CD4 Tregs within tumors was more pronounced than in spleen, again suggesting that Helios expression by Tregs might be particularly important for stable FoxP3 expression within the chronic inflammatory environment of growing tumors. In accord with FoxP3 down-regulation, intratumoral Helios-deficient CD4 Tregs expressed high levels of IFNγ, suggesting a Treg→Teff cell conversion within the tumor (FIG. 34C). Moreover, intratumoral CD4 and CD8 Teff cells in adoptive hosts transferred with Helios KO CD4 Tregs displayed increased IFNγ expression (FIG. 34D).

Helios Deficiency Promotes in Vitro Conversion of Tregs into Teff Cells.

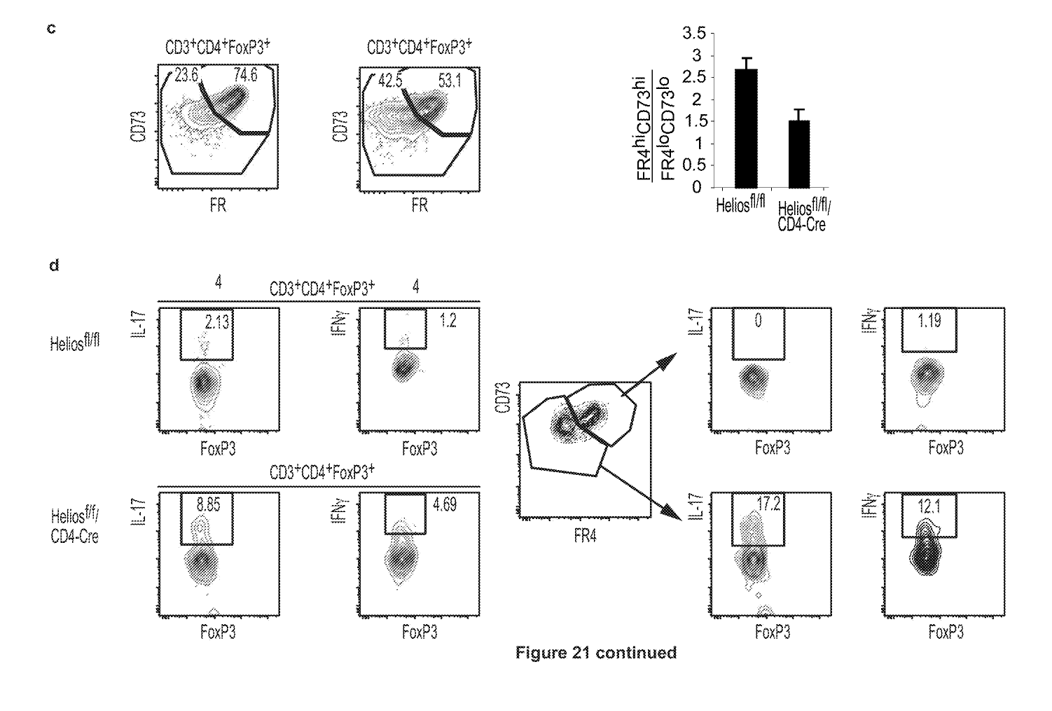

To further investigate the basis for the intrinsic instability of Helios-deficient CD4 Tregs, an in vitro system that allows analysis of Treg stability in the presence of inflammatory cytokines IL-2/IL-4 was used (6). Here, the responses of Helios^{+/+} and Helios^{-/-} CD4 Tregs to graded concentrations of IL-2 and the proinflammatory cytokine IL-4 were analyzed (FIG. 35A). Helios-deficient Tregs displayed profoundly reduced expression of both FoxP3 and CD25 and enhanced expression of IFNγ in an IL-2 dose-dependent manner (FIG. 35A); Helios WT CD4 Tregs expressed high levels of FoxP3 and CD25 that were not affected by IL-2 concentrations. Although Helios-deficient CD4 Tregs showed a significant increase in CD25 expression in the presence of higher concentrations of IL-2, it is unlikely that increased CD25 expression accounted for cytokine conversion, because FoxP3^{lo} cells marked by low CD25 expression produced the highest levels of IFNγ (FIG. 35B). Taken together, these findings indicate that Helios makes an important contribution to the stability and survival of FoxP3⁺CD4 Tregs in the presence of inflammatory cytokines in this in vitro assay. The dependence of CD4 Tregs on IL-2 for survival and lineage stability reflects an interaction between STAT5 and the FoxP3 intronic CNS2 region that promotes stable FoxP3 expression (12). These findings confirm that the unstable phenotype of Helios-deficient Tregs can be induced by blockade of STAT5 activation. The AG-490 STAT5 inhibitor reduced Treg survival, diminished FoxP3 expression, and enhanced IFNγ effector cytokine expression (FIG. 5C). These data suggest that approaches that down-regulate Helios in vivo can enhance tumor immunity via reduction of CD4 Treg numbers and conversion of a portion of the surviving Tregs into an effector cell phenotype.

### Engagement of Glucocorticoid-Induced TNF Receptor Induces Helios Down-Regulation by CD4 Treg and Enhanced Antitumor Immunity.

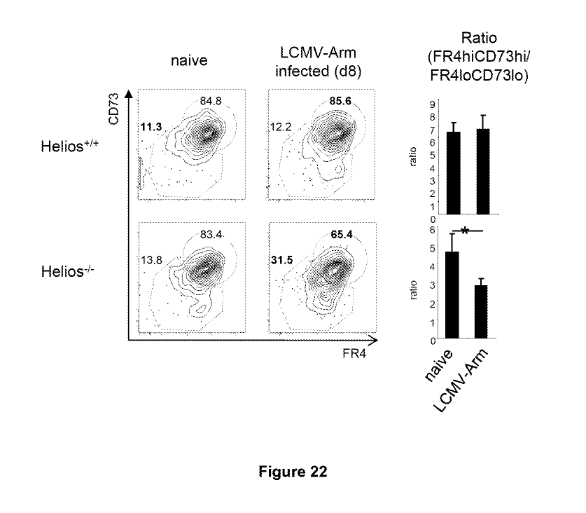

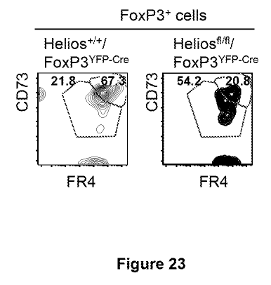

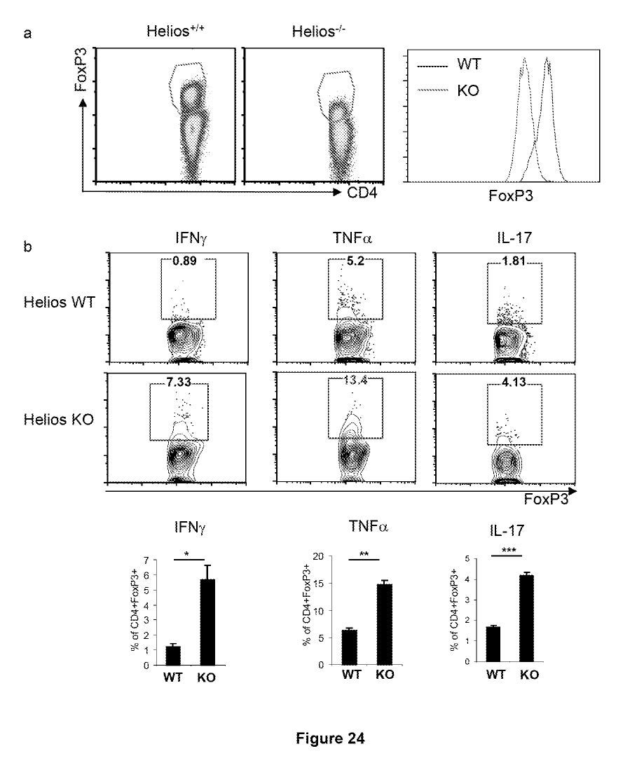

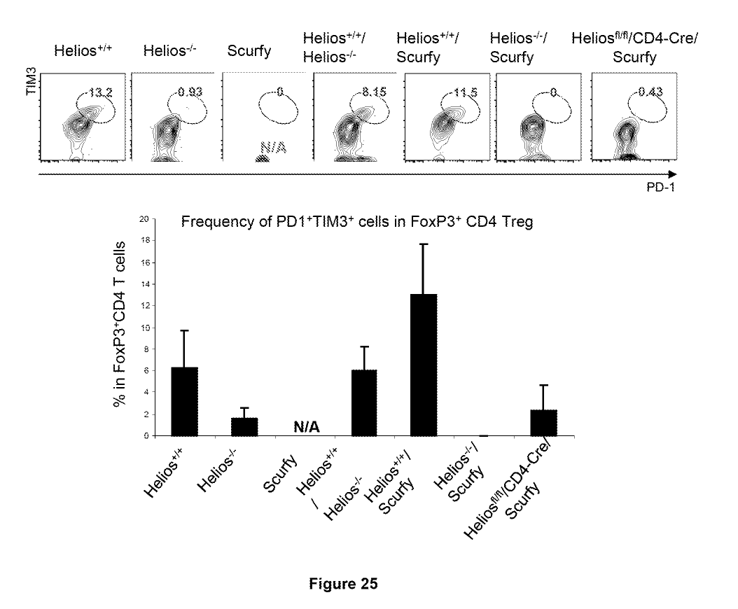

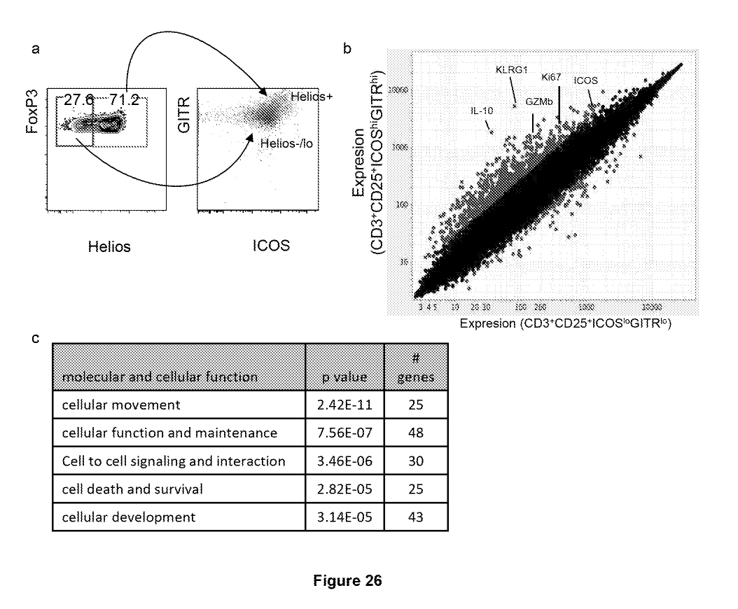

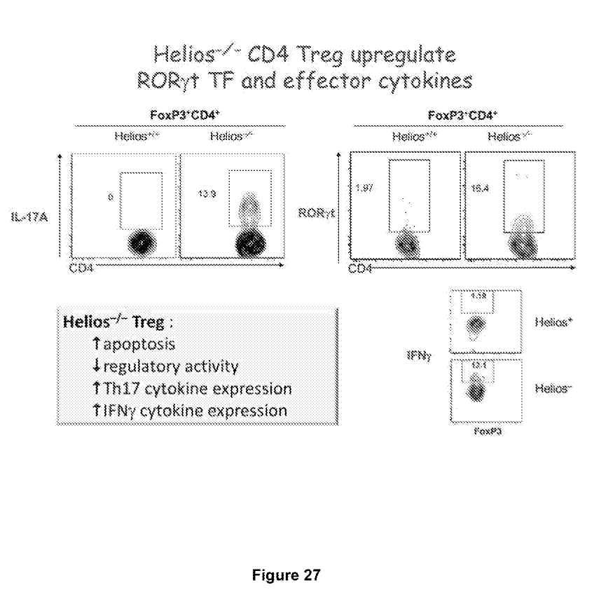

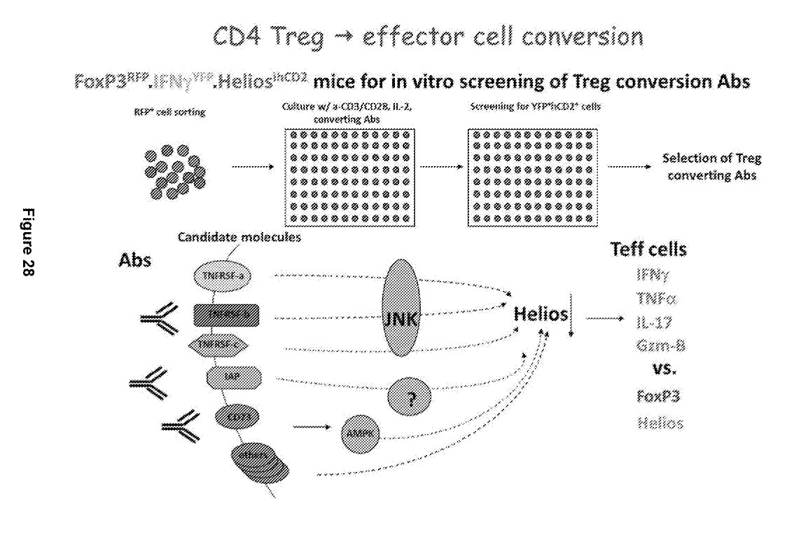

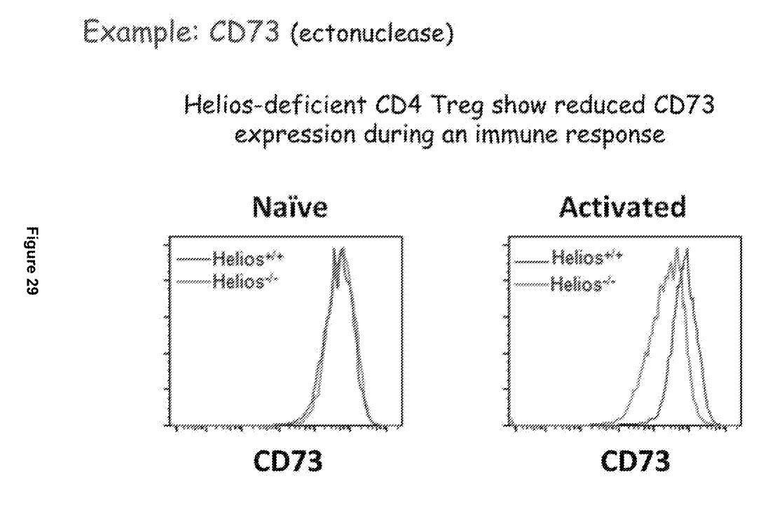

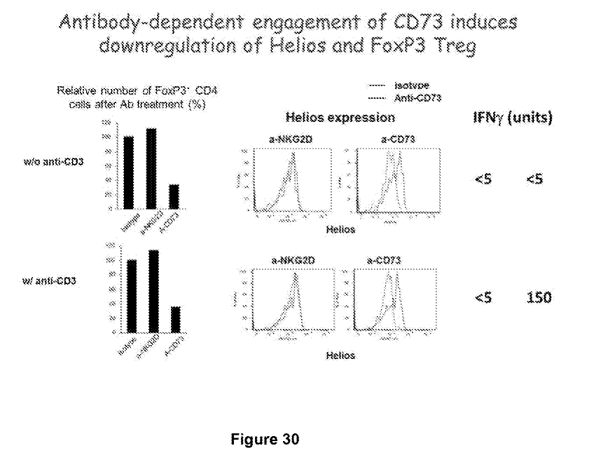

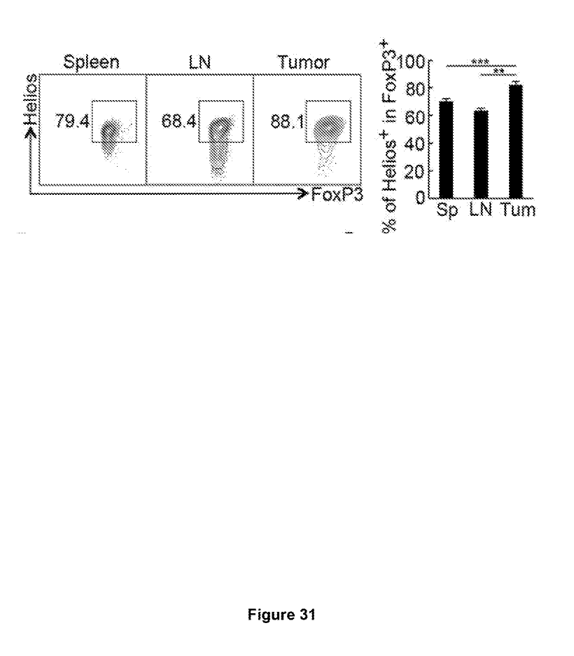

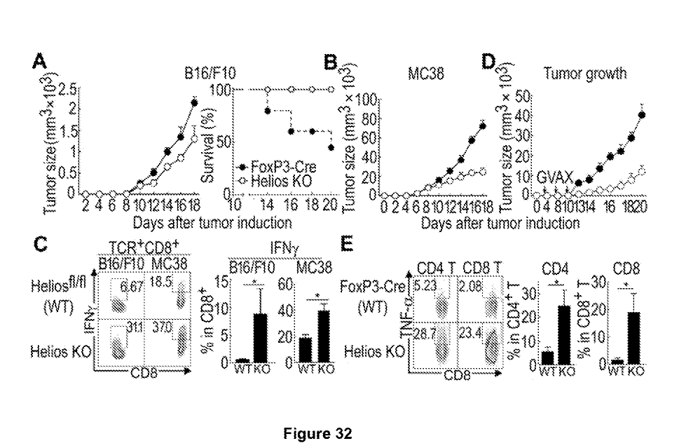

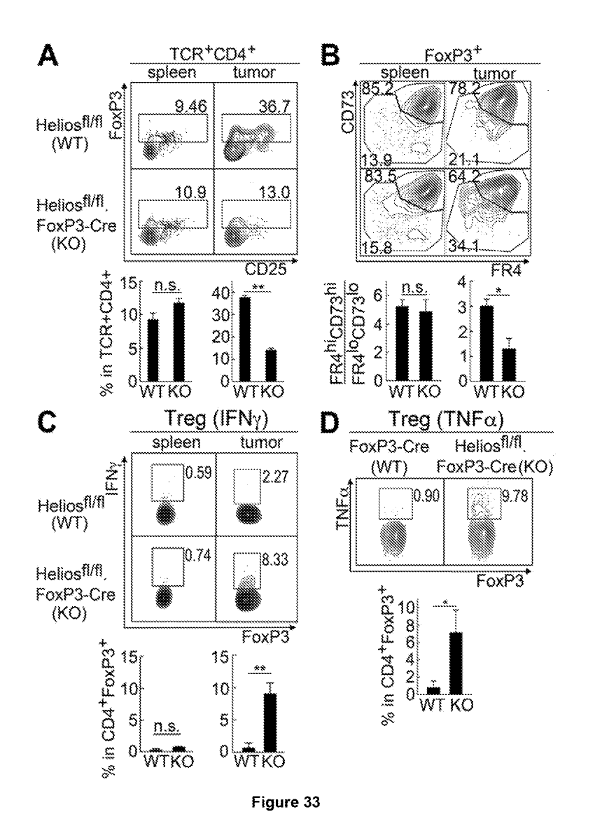

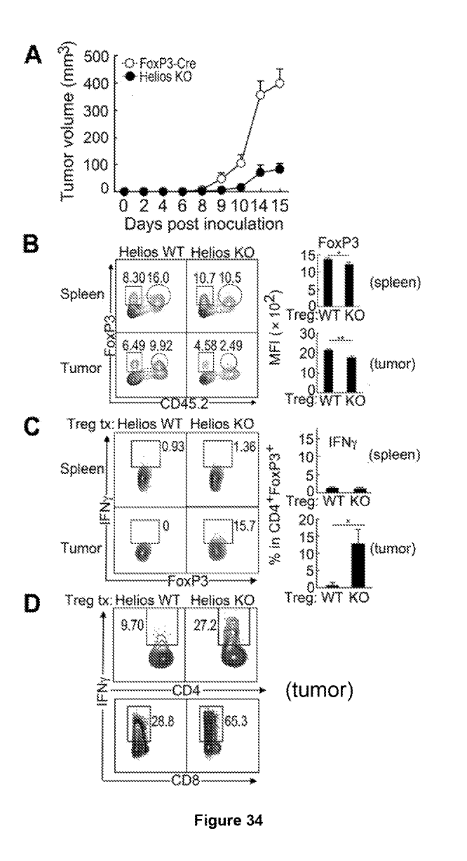

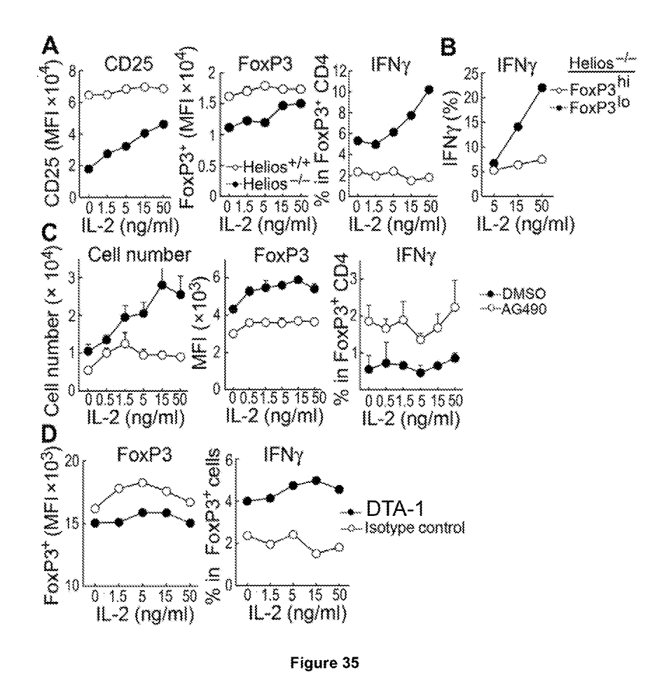

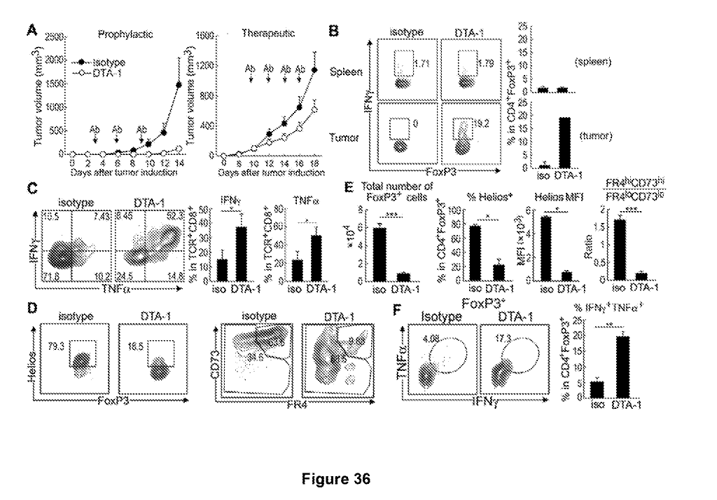

The observation that Helios-deficient CD4 Treg convert to Teff cells within the TME and enhance antitumor responses opened the possibility that an immunotherapeutic regimen that induces Helios down-regulation might be exploited to enhance antitumor immunity. To this end, an in vitro Treg conversion assay was used (FIG. 5D) to screen for antibodies that induced conversion by Helios WT Tregs. One antibody identified was specific for glucocorticoidinduced TNF receptor (GITR), a member of the TNF receptor gene family that is prominently expressed by CD4 Tregs compared with other T cells that normally display low expression levels (13). Engagement of GITR on CD4 Tregs by antibodies in vitro in the presence of proinflammatory cytokine IL-4 resulted in induction of an unstable Treg phenotype characterized by reduced FoxP3 expression and IFNγ production (FIG. 35D). The agonistic antibody DTA-1 has been shown to diminish CD4 Treg activity and enhance antitumor immunity, which has been attributed in part to decreased Treg lineage stability within tumors (14). Therefore whether engagement of GITR results in Helios down-regulation and enhanced antitumor immunity was tested. Although repeated administration of a relatively low dose of DTA-1 (200 μg) did not induce obvious side effects (15), both prophylactic and therapeutic regimens significantly delayed tumor growth (FIG. 36A) and were associated with diminished expression of Helios and increased IFNγ production by intratumoral CD4 Tregs (FIG. 36B). Moreover, CD8 Teff cells isolated from tumors in DTA-1—treated mice displayed increased IFNγ and TNFα production compared with CD8 Teff cells from isotype-control-treated mice (FIG. 36C). The phenotypic changes in CD4 Tregs after DTA-1 treatment can reflect a contribution of Teff cells that express GITR after activation. To more directly analyze the isolated effects of DTA-1 on CD4 Tregs, purified CD4 Tregs (YFP⁺ from FoxP3YFP-Cre mice) were transferred into Rag2^{-/-} hosts before treatment with DTA-1 or control Ig. Tregs in DTA-1—treated adoptive hosts showed a profound decrease in Helios expression, acquisition of a nonanergic phenotype, and reduced survival (FIGS. 36D and 36E). Moreover, ˜20% of the (Helios^{lo}) FoxP3⁺CD4 Tregs from DTA-1-treated mice produced IFNγ and TNFα effector cytokines, consistent with Treg-Teff conversion (FIG. 6F). However, the dramatic reduction in the anergic phenotype by DTA-1-treated CD4 Tregs indicates the impact of DTA-1 on the reactivity of Tregs is not limited to up-regulation of TNF-α/IFNγ. Together, these data suggest that the enhanced antitumor immunity after administration of DTA-1 reflects, at least in part, induction of an unstable CD4 Treg phenotype and Treg conversion. These data also suggest that Helios may represent a previously unrecognized target for cancer immunotherapy in light of its impact on intratumoral Treg stability.

### Discussion of Experimental Results

The definition of immunoinhibitory pathways that are up-regulated after T-cell activation has led to significant insight into tumor escape mechanisms. Many of these findings have been incorporated into approaches that combine checkpoint blockade with immunostimulatory agents that can promote sustained antitumor immune responses (16). In some cases, these approaches also may affect Treg function. For example, the antitumor activity of some anti-CTLA-4 antibodies may reflect FcγR-dependent depletion of intratumoral Tregs in addition to targeting of Teff cells (17). Likewise, PD-1-based approaches may affect both Treg suppressive activity as well as effector T-cell responses (18). Because depletion of Treg activity may also produce adverse autoimmune side effects (19), approaches that preferentially target intratumoral Tregs without affecting the Treg systemic phenotype potentially represent a more effective strategy for cancer immunotherapy. The data discussed above show that a selective deficiency of Helios in FoxP3⁺CD4 Tregs results in increased Treg instability and conversion of intratumoral CD4 Treg to Teff cells and enhanced antitumor immunity. Instability of intratumoral Tregs can increase the numbers of Teff cells within tumors as a combined result of Treg conversion and reduced Treg suppressive activity. In addition, defective IL-2 responses of Helios-deficient intratumoral Tregs resulting in decreased numbers of activated Tregs can also contribute to increased intratumoral effector T-cell activity. Interaction between tumor cells and infiltrating immune cells results in secretion of inflammatory mediators, including TNF-α, IL-6, IL-17, IL-1, and TGF-β, and the formation of a local inflammatory environment. Although the signaling pathway(s) that leads to effector cell conversion of Helios-deficient Tregs has not been identified, cytokine-mediated inflammation, competition for limited amounts of IL-2, and hypoxic conditions within the TME may promote conversion within the TME but not peripheral tissues, perhaps by skewing the pSTAT5/pSTAT3 ratio bound to Treg-specific demethylated regions (6, 12). Because conversion of Helios-deficient Tregs occurs within the local inflammatory environment of the tumor (e.g., FIG. 33A-FIG. 33C and FIG. 34A-FIG. 34D), this approach may not provoke the autoimmune side effects associated with systemic reduction of Tregs (12, 16). Although thymic-derived Tregs that recognize tissue-specific antigens expressed by tumors and their parent tissues may be highly represented within tumors (20), under normal conditions, this autoreactive TCR repertoire does not translate into robust antitumor responses. The data presented above suggest that this tumor recognition bias of Treg may be exploited by approaches that induce Treg conversion into MHC class II/peptide-reactive effector cells that directly kill tumor cells (21-23). These considerations suggest that protocols for transfer of TAA-specific CD4 T cells can benefit from approaches that down-regulate Helios expression by CD4 Tregs to obtain increased antitumor reactivity from both conventional CD4 cells and Helios-deficient converted Tregs. This study also suggests that Treg-Teff conversion of Helios deficient Tregs within the local inflammatory setting of growing tumors can be mimicked by Antibody-dependent engagement of surface receptors that down-regulate Helios expression. An in vitro screen of antibodies that might reduce Helios expression and enhance Treg conversion suggested the contribution of GITR to this process. The impact of GITR Antibodies on tumor immunity has been described and may depend in part on engagement of GITR⁺ Tregs (14). We note that this TNFR costimulatory molecule, which is constitutively and highly expressed on Tregs and induced after activation of Teff cells, induces Helios down-regulation and Treg conversion that is restricted to tumor sites. The CD4 Treg transfer experiments also support the idea that potentiation of antitumor immunity by anti-GITR antibody administration can be attributed largely to induction of an unstable Treg phenotype. Administration of anti-GITR antibody can lead to untoward side effects in mice (15); however, relatively low doses of anti-GITR antibody (200 μg) suffice to induce relatively selective Treg conversion, in view of the relatively low GITR levels expressed by activated Teff cells (13). These findings suggest that administration of relatively low doses of anti-GITR Ab to impair Treg activity, perhaps in combination with T-cell-activating immunotherapy, may yield strong antitumor immunity.

To summarize, depletion or inhibition of regulatory T cells (Tregs) has been associated with increased effector T-cell activation that may enhance antitumor responses. A potentially more effective strategy depends on induction of lineage instability that allows conversion of intratumoral but not systemic Tregs into effector T cells (Teffs). The data described above show that targeted deletion of the Helios transcription factor within CD4 Tregs promotes instability and effector cell conversion of Tregs in tumors and increased antitumor immunity. Antibody-dependent ligation of Treg surface receptors that diminishes Helios expression can also induce intratumoral Treg conversion. These findings indicate that targeting of signaling pathways that reduce Helios expression by intratumoral Tregs represent a potentially robust approach to cancer immunotherapy.

### Materials and Methods

### Mice and Treatment.

All mice were purchased from The Jackson Laboratory or Taconic Farms and maintained in specific pathogen-free conditions in the Dana-Farber Cancer Institute (DFCI) Animal Resource Facility. C57BL/6J (B6) and B6.129(Cg)-Foxp3^{tm4(YFP/icre)Ayr}/J (FoxP3^{YFP}-Cre) mice were purchased from The Jackson laboratory. B6.129S6-Rag2^{tm1Fwa} N12 (B6 Rag2^{-/-}) and B6.SJL-Ptprc^{a}/BoyAiTac (B6 CD45.1⁺) were from Taconic Farms. Ikzf2^{fl/fl} (Helios^{fl/fl}) mice, which bear a loxP-flanked Helios allele, were kindly provided by Ethan Shevach (25). Helios^{fl/fl}.FoxP3^{YFP}-Cre mice were generated by crossing Helios^{fl/fl} mice to FoxP3^{YFP}-Cre mice.

For tumor induction in B16/F10 and MC38 transplantable tumor models, mice were injected with 2×10⁵ cells s.c. in the right flank. In the B16-GVAX model, mice were injected s.c. with 2×10⁵ B16-OVA cells in the right flank followed by vaccination with 1×10⁶ irradiated (150 Gy) B16-GMCSF cells on the contralateral flank at day 3, 6, and 9. Tumor growth was monitored every 2 d and tumor volume was calculated by: tumor volume (mm3)=longest diameter (mm)×shortest diameter (mm)×width (mm)/2. All animal protocols in this study were approved by DFCI's Animal Care and Use Committee, and all animal experiments were performed in compliance with federal laws and institutional guidelines.

### DNA Microarray.

Helios⁺ and Helios CD4⁺CD25⁺ T cells were separated by sorting cells after staining with surrogate markers ICOS and GITR (ICOS^{hi}GITR^{hi}: Helios⁺; ICOSloGITR^{lo}: Helios^{-/lo}). RNA was prepared with the RNeasy mini kit (Qiagen). RNA amplification, labeling, and hybridization to MOA430 2.0 chips (Affymetrix) were performed at the DFCI Molecular Biology Core Facility. Relative gene expression by ICOS^{hi}GITR^{hi} and ICOS^{lo}GITR^{lo} cells was analyzed by the Multiplot program.

### Cell Lines.

B16/F10 melanoma cells were purchased from American Type Culture Collection. B16-OVA and B16-GVAX (B16-GM-CSF) were maintained in complete Dulbecco's Modified Eagle Medium (DMEM; Thermo Fisher Scientific) containing 10% (vol/vol) FCS (Sigma-Aldrich) and 250 μg/mL of G418 (Thermo Fisher Scientific). MC38 colon cancer cells were cultured in complete RPMI-1640 (Sigma-Aldrich) containing 10% (vol/vol) FCS. All tumor cell lines were maintained at 37° C. with 5% CO₂.

### Isolation of Tumor-Infiltrating Lymphocytes.

Mice were killed before tumor sizes reached 2,000 mm³ and analyzed. Harvested tumors were mechanically chopped and dispersed into small pieces followed by collagenase digestion for 1 h with 50 units/mL collagenase type I (Thermo Fisher Scientific) and 20 units/mL DNase I (Roche). Digested samples were filtered and enriched for tumor-infiltrating lymphocytes by centrifugation through a Ficoll-Paque 1.084 density gradient (GE Healthcare).

### Flow Cytometry and Cell Sorting.

Fluorescence dye conjugated monoclonal antibodies specific for CD4 (RM4-5), CD8 (53-6.7), CD25 (PC61), TCR V_{β} (H57-597), FoxP3 (FJK-16s), Helios (22F6), GITR (DTA-1), ICOS (7E.17G9), IFNγ (XMG1.2), TNFα (MP6-XT22), FR4 (12A5), CD73 (TY/11.8), and CD45.1 (A20) were purchased from BD Bioscience, eBioscience, or BioLegend. IFNγ and TNFα were detected after restimulation of cells in vitro with leukocyte activation mixture with BD Golgi-Plug (BD Bioscience) for 5 h. Stimulated cells were stained for surface markers first, then fixed, permeabilized using the FoxP3 staining buffer set (eBioscience) and stained with antibodies for cytokines. Samples were measured by BD LSRFortessa X-20 (BD Bioscience) and data were analyzed using FlowJo v10 (FlowJo). For CD4 Treg isolation, cells were enriched CD4⁺CD25⁺ cells using a CD4 Treg enrichement kit (Miltenyi) followed by sorting for CD4 Tregs using BD FACSAria IIIu (BD Bioscience).

### Antibody Treatment.

Anti-GITR monoclonal antibody (clone: DTA-1) and isotype control (Rat IgG2b clone: LTF-2) were purchased from Bioxcell. For prophylactic treatment, 200 μg of antibody was i.v. injected into the tail vein of mice at day 0, 3, 6, and 9 after tumor cell injection.

### Cell Purification and Adoptive Transfer.

CD4⁺CD25⁻ effector cells were negatively isolated from spleens of CD45.1 mice using a Mouse CD4 T Lymphocyte Enrichment Set supplemented with biotinylated anti-CD25 antibodies (BD Bioscience). CD8⁺Ly49⁻ effector cells were negatively isolated from spleens of CD45.1 mice using a Mouse CD8 T Lymphocyte Enrichment Set (BD Bioscience) supplemented with biotinylated anti-Ly49C/I/F/H antibodies (14B11). Purity of CD4 and CD8 cells was >90%. CD4 Tregs were obtained from spleens of Helios^{fl/fl}.FoxP3^{YFP}-Cre and FoxP3^{YFP}-Cre (Helios WT) mice by sorting TCR⁺CD4⁺YFP⁺ cells after enrichment using a CD4⁺CD25⁺ Regulatory T Cell Isolation Kit (Miltenyi Biotec). CD4 Treg purity was >95%. The 5×10⁵ CD4 Tregs were transferred i.v. into Rag2^{-/-} hosts along with 2×10⁶ CD4 and 1×10⁶ CD8 T effector cells on day 0. To establish tumors, 2×10⁵ MC38 tumor cells were inoculated s.c. on day 2, and tumor growth was monitored.

### In Vitro Stimulation of CD4 Tregs.

CD4 Tregs were isolated from Helios^{+/+} and Helios^{-/-} mice using a CD4⁺CD25⁺ Regulatory T Cell Isolation Kit (Miltenyi Biotec) followed by sorting for CD4⁺CD25⁺ cells. CD4 Treg purity was >95%. Sorted CD4 Treg were cultured on a 96-well flat bottom plate coated with anti-CD3 (17A2, eBioscience) and anti-CD28 antibody (37.51, eBioscience) in the presence of IL-4 (20 ng/mL) and IL-2 (0-50 ng/mL) (eBioscience) for 4-5 d before flow cytometry analysis. For the in vitro STAT5 inhibition assay, isolated CD4 Tregs were cultured with DMSO or AG490 (50 μM) (Sigma-Aldrich).

CD4 Treg Transfer into Rag2^{-/-} Mice and Antibody Treatment.

CD4 Tregs were isolated from FoxP3^{YFP}-Cre (Helios WT) mice as described above and transferred into Rag2^{-/-} hosts. DTA-1 or isotype antibodies were injected i.v. via tail vein on days 0, 7, 14, and 20. Spleens were harvested on day 21 and analyzed by flow cytometry.

### Statistical Analysis.

Statistical significance was calculated according to the Wilcoxon-Mann-Whitney rank sum test. A P value of <0.05 was considered to be statistically significant (*P<0.05, **P<0.01, ***P<0.001).

### REFERENCES

1. Scanlan M J, Gure A O, Jungbluth A A, Old L J, & Chen Y T (2002) Cancer/testis antigens: an expanding family of targets for cancer immunotherapy. Immunol Rev 188:22-32.
2. Nishikawa H & Sakaguchi S (2014) Regulatory T cells in cancer immunotherapy. Curr Opin Immunol 27:1-7.
3. Kurtulus S, et al. (2015) TIGIT predominantly regulates the immune response via regulatory T cells. J Clin Invest 125(11):4053-4062.
4. Sugiyama D, et al. (2013) Anti-CCR4 mAb selectively depletes effector-type FoxP3+CD4+ regulatory T cells, evoking antitumor immune responses in humans. Proc Natl Acad Sci USA 110(44):17945-17950.
5. Yamaguchi T, et al. (2007) Control of immune responses by antigen-specific regulatory T cells expressing the folate receptor. Immunity 27(1):145-159.
6. Kim H J, et al. (2015) Stable inhibitory activity of regulatory T cells requires the transcription factor Helios. Science 350(6258):334-339.
7. Sebastian M, et al. (2016) Helios Controls a Limited Subset of Regulatory T Cell Functions. J Immunol 196(1):144-155.
8. Liston A & Gray DH (2014) Homeostatic control of regulatory T cell diversity. Nat Rev Immunol.
9. Sakuishi K, et al. (2013) TIM3FOXP3 regulatory T cells are tissue-specific promoters of T-cell dysfunction in cancer. Oncoimmunology 2(4):e23849.
10. Coussens L M & Werb Z (2002) Inflammation and cancer. Nature 420(6917):860-867.
11. Cai Q, Dierich A, Oulad-Abdelghani M, Chan S, & Kastner P (2009) Helios deficiency has minimal impact on T cell development and function. Journal of Immunology 183(4):2303-2311.
12. Feng Y, et al. (2014) Control of the inheritance of regulatory T cell identity by a cis element in the Foxp3 locus. Cell 158(4):749-763.
13. Shimizu J, Yamazaki S, Takahashi T, Ishida Y, & Sakaguchi S (2002) Stimulation of CD25+CD4+ regulatory T cells through GITR breaks immunological self-tolerance. Nat. Immunol 3(2):135-142.
14. Schaer D A, et al. (2013) GITR pathway activation abrogates tumor immune suppression through loss of regulatory T cell lineage stability. Cancer Immunol Res 1(5):320-331.
15. Murphy J T, et al. (2014) Anaphylaxis caused by repetitive doses of a GITR agonist monoclonal antibody in mice. Blood 123(14):2172-2180.
16. Mahoney K M, Rennert P D, & Freeman G J (2015) Combination cancer immunotherapy and new immunomodulatory targets. Nature reviews. Drug discovery 14(8):561-584.
17. Simpson T R, et al. (2013) Fc-dependent depletion of tumor-infiltrating regulatory T cells co-defines the efficacy of anti-CTLA-4 therapy against melanoma. J Exp Med 210(9):1695-1710.
18. Francisco L M, et al. (2009) PD-L1 regulates the development, maintenance, and function of induced regulatory T cells. J Exp Med 206(13):3015-3029.
19. Page D B, Postow M A, Callahan M K, Allison J P, & Wolchok J D (2014) Immune modulation in cancer with antibodies. Annual review of medicine 65:185-202.
20. Malchow S, et al. (2013) Aire-dependent thymic development of tumor-associated regulatory T cells. Science 339(6124):1219-1224.
21. Quezada S A, et al. (2010) Tumor-reactive CD4+ T cells develop cytotoxic activity and eradicate large established melanoma after transfer into lymphopenic hosts. Journal of Experimental Medicine 207:637-650.
22. Hirschhorn-Cymerman D, et al. (2012) Induction of tumoricidal function in CD4+ T cells is associated with concomitant memory and terminally differentiated phenotype. J Exp Med 209(11):2113-2126.
23. Malandro N, et al. (2016) Clonal Abundance of Tumor-Specific CD4+ T Cells Potentiates Efficacy and Alters Susceptibility to Exhaustion. Immunity 44(1):179-193.
24. Xiao X, et al. (2015) GITR subverts Foxp3+ Tregs to boost Th9 immunity through regulation of histone acetylation. Nature communications 6:8266.
25. Thornton A M, et al. (2010) Expression of Helios, an Ikaros transcription factor family member, differentiates thymic-derived from peripherally induced Foxp3+T regulatory cells. Journal of Immunology 184:3433-3441.

1. A method for modulating differentiation of a regulatory CD4⁺ T (CD4⁺ Treg) cell to a CD4⁺ effector T cell, the method comprising contacting the CD4⁺ Treg with an agent that modulates Helios activity and/or Helios expression, wherein:
(i) differentiation is induced by an agent that decreases Helios activity and/or Helios expression; and/or
(ii) differentiation is inhibited by an agent that increases Helios activity and/or Helios expression.

2. The method of claim 1, wherein the CD4⁺ Treg cell is FoxP3⁺ and CD25⁺.

3. The method of claim 1, wherein the agent is selected from the group consisting of peptide, polypeptide, small molecule, antibody, and RNAi molecule.

4. The method of claim 3, wherein the agent is an antibody.

5. The method of claim 4, wherein the antibody is selected from the group consisting of anti-GITR, anti-OX-40, anti-CD47, anti-4-1BB, anti-Nrp-1, and anti-CD73 antibody.

6. The method of claim 3, wherein the small molecule is a zinc finger protein inhibitor.

7. The method of claim 1, wherein the CD4⁺ effector T cell expresses one or more effector cytokines.

8. The method of claim 7, wherein the effector cytokines are selected from the group consisting of tumor necrosis factor alpha (TNF-α), interferon-γ (IFN-γ), interleukin-17 (IL-17), interleukin-2 (IL-2), and Granzyme B.

9. A method for modulating differentiation of a regulatory CD8⁺ T (CD8⁺ Treg) cell to a CD8⁺/PD1⁺/TIM3⁺ T cell, the method comprising contacting the regulatory T cell with an agent that modulates Helios activity and/or Helios expression; wherein
(i) differentiation is induced by an agent that decreases Helios activity and/or Helios expression; and/or
(ii) differentiation is inhibited by an agent that increases Helios activity and/or Helios expression.

10. The method of claim 9, wherein the CD8⁺ Treg cell is Kir⁺.

11. The method of claim 9, wherein the agent is selected from the group consisting of peptide, polypeptide, antibody small molecule and RNAi molecule.

12. The method of claim 11, wherein the agent is an antibody.

13. The method of claim 12, wherein the antibody is selected from the group consisting of anti-Kir, anti-Ly49F, or a bispecific anti-CD8/anti-Kir antibody.

14. The method of claim 13, wherein the small molecule is a zinc finger protein inhibitor, or a Stat5b inhibitor.

15. The method of claim 9, wherein the CD8⁺/PD1⁺/TIM3⁺ T cell express increased levels of BLIMP-1 transcription factor when compared to wild-type CD8⁺ regulatory T cells.

16-41. (canceled)

42. A method for identifying candidate compounds for modulating Helios activity and/or Helios expression, the method comprising:
(a) contacting a regulatory T cell with a test compound;
(b) measuring Helios activity level and/or Helios expression level in the cell;
(c) identifying the test compound as a candidate compound for modulating Helios activity and/or Helios expression if the Helios activity level and/or Helios expression level is increased or decreased relative to a control cell that has been treated with a compound known to not modulate Helios activity level and/or Helios expression level.

43-51. (canceled)

52. The method of claim 1, wherein the contacting is in vitro or in vivo.

53. The method of claim 9, wherein the contacting is in vitro or in vivo.

54. The method of claim 42, wherein the test compound is identified as a candidate compound for decreasing Helios activity and/or Helios expression if the Helios activity level and/or Helios expression level is decreased relative to a control cell that has been treated with a compound known to not decrease Helios activity level and/or Helios expression level.
